SEQUENCE LISTING (22 sequences)

SEQ ID NO: 1 - protein, 304 aa, SARS
SGFRKMAFPSGKVEGCMVQVTCGTTTLNGLWLDDTVYCPRHVICTAEDMLNPNYEDLLIRKSNHSFLVQAGNVQLRVIGHSMQNCLLRLKVDTSNPKTPKYKFVRIQPGQTFSVLACYNGSPSGVYQCAMRPNHTIKGSFLNGSCGSVGFNIDYDCVSFCYMHHMELPTGVHAGTDLEGKFYGPFVDRQTAQAAGTDTTITLNVLAWLYAAVINGDRWFLNRFTTTLNDFNLVAMKYNYEPLTQDHVDILGPLSAQTGIAVLDMCAALKELLQNGMNGRTILGSTILEDEFTPFDVVRQCSGVT

SEQ ID NO: 2 - protein, 301 aa, MHV Protease
SGIVKMVSPTSKVEPCVVSVTYGNMTLNGLWLDDKVYCPRHVICSSADMTDPDYPNLLCRVTSSDFCVMSDRMSLTVMSYQMQGSLLVLTVTLQNPNTPKYSFGVVKPGETFTVLAAYNGRPQGAFHVVMRSSHTIKGSFLCGSCGSVGYVLTGDSVRFVYMHQLELSTGCHTGTDFSGNFYGPYRDAQVVQLPVQDYTQTVNVVAWLYAAILNRCNWFVQSDSCSLEEFNVWAMTNGFSSIKADLVLDALASMTGVTVEQVLAAIKRLHSGFQGKQILGSCVLEDELTPSDVYQQLAGVK

SEQ ID NO: 3 - protein, 40 aa, SARS-HCV
GSPSGVYQCAMRPNHTIKGSFLNGSCGSVGFNIDYDCVSF

SEQ ID NO: 4 - protein, 40 aa, MHV ML-10
GRPQGAFHVTLRSSHTIKGSFLCGSCGSVGYVLTGDSVRF

SEQ ID NO: 5 - protein, 40 aa, MHV A59; identical to SEQ ID NO: 4

SEQ ID NO: 6 - protein, 40 aa, MHV JHM
GRPQGAFHVVMRSSHTIKGSFLCGSCGSVGYVLTGDSVRF

SEQ ID NO: 7 - protein, 40 aa, MHV-2
GKSQGAFHVTMRSSYTIKGSFLCGSCGSVGYVLTGDSVRF

SEQ ID NO: 8 - protein, 40 aa, MHV Penn 97-1; identical to SEQ ID NO: 7

SEQ ID NO: 9 - protein, 40 aa, MHV ML-11; identical to SEQ ID NO: 7

SEQ ID NO: 10 - protein, 40 aa, BCV Quebec
GKPQGAFHVTMRSSYTIKGSFLCGSCGSVGYVIMGDCVKF

SEQ ID NO: 11 - protein, 40 aa, BCV LUN
GKPQGAFHVTMRSSYTIKGSFLCGSCGSVGYVLMGDCVKF

SEQ ID NO: 12 - protein, 40 aa, BCV Mebus
GKPQGAFHVTMRSSYTIKGSFLCGSCGSVVYVIMGDCVKF

SEQ ID NO: 13 - protein, 40 aa, BCV ENT; identical to SEQ ID NO: 11

SEQ ID NO: 14 - protein, 40 aa, PEDV-CV777
GAAAGVYGVNMRSNYTIRGSFINGACGSPGYNINNGTVEF

SEQ ID NO: 15 - protein, 40 aa, TGEV Purdue-115
GCPGSVYGVNMRSQGTIKGSFIAGTCGSVGYVLENGILYF

SEQ ID NO: 16 - protein, 40 aa, TGEV PUR46-MAD; identical to SEQ ID NO: 15

SEQ ID NO: 17 - protein, 40 aa, FIPV 79-1146
GCPGSVYGVNMRSQGTIKGSFIAGTCGSVGYVLENGTLYF

SEQ ID NO: 18 - protein, 40 aa, HCV 229E
GCAQGVFGVNMRTNWTIRGSFINGACGSPGYNLKNGEVEF

SEQ ID NO: 19 - protein, 40 aa, IBV Beaudette
GTVVGLYPVTMRSNGTIRASFLAGACGSVGFNIEKGVVNF

SEQ ID NO: 20 - protein, 40 aa, IBV LX4
GTVIGLYPVTMRSNGTIRASFLAGACGSVGFNIERGVVNF

SEQ ID NO: 21 - protein, 40 aa, IBV Beaudette CK; identical to SEQ ID NO: 19

SEQ ID NO: 22 - protein, 40 aa, IBV BJ
GTVIGLYPVTMRSNGTIRASFLAGACGSVGFNIEKGVVNF

(19) United States
(12) Patent Application Publication
(10) Pub. No.: US 2005/0267071 A1
(43) Pub. Date: Dec. 1, 2005

(54) Inhibitors of coronavirus protease and methods of use thereof

(75) Inventors: Ernesto Freire, Baltimore, MD (US); Raphael Ottenbrite, Midlothian, VA (US); Yingxin Xiao, Gaithersburg, MD (US); Adrian Velazquez-Campoy, Zaragoza (ES); Stephanie Leavitt, Belmont, CA (US); Usman Bacha, Baltimore, MD (US); Jennifer Barrila, Baltimore, MD (US)

Correspondence Address: DARBY & DARBY P.C., P. O. BOX 5257, NEW YORK, NY, 10150-5257, US

(73) Assignees: Fulcrum Pharmaceuticals, Inc., Las Vegas, NV (US); Johns Hopkins University, Baltimore, MD (US)

(21) Appl. No.: 10/979,871

(22) Filed: Nov. 1, 2004

Related U.S. Application Data
(60) Provisional application No. 60/516,008, filed on Oct. 31, 2003.

(51) Int. Cl. A61K 31/69; C07F 5/04; C07F 5/02

(52) U.S. Cl. 514/64; 549/4; 562/7

(57) ABSTRACT

This invention provides organic boron-containing compounds, compositions thereof, and methods of using such compounds and compositions for inhibiting coronavirus protease(s) and for treating infections.

### CROSS REFERENCE TO RELATED APPLICATIONS

The present application claims priority under 35 U.S.C. § 119(e) of provisional application 60/516,008, filed Oct. 31, 2003, which is hereby incorporated herein by reference in its entirety.

### STATEMENT OF GOVERNMENT INTEREST

A portion of this invention was made with support under Grant No. GM-57144, awarded by the National Institutes of Health. The United States Government may have certain rights in this invention.

### FIELD OF THE INVENTION

The present invention relates to boron-containing compounds that are inhibitors of coronavirus protease and methods of use thereof.

### BACKGROUND OF THE INVENTION

Severe Acute Respiratory Syndrome (SARS)

The first cases of Severe Acute Respiratory Syndrome (SARS) appeared at the end of 2002 in Southern China. By May 2003, SARS had spread to other continents through international travel. It is estimated by the World Health Organization that a total of 15,000 people were infected during the outbreak with an average mortality rate of 15%. The actual mortality rate appears to depend on the age of the patient. The fatality ratio is estimated to be less than 1% in persons aged 24 years or younger, 6% in persons aged 25 to 44 years, 15% in persons aged 45 to 64 years, and greater than 50% in persons aged 65 years and older.

SARS patients typically have high fever, malaise, rigor, headache and nonproductive cough or dyspnea and may progress to generalized interstitial infiltrated in the lung, requiring incubation and mechanical ventilation.

The causative agent of SARs is a coronavirus never before seen in humans. The genome of the SARS-associated coronavirus has been sequenced. The genome sequence of the SARS-associated coronavirus reveals that the virus does not belong to any of the known groups of coronaviruses, including two human coronaviruses, HCoV-OC43 and HCoV-229E (Drosten et al., Identification of a Novel Coronavirus in Patients with Severe Acute Respiratory Syndrome. N. Engl. J. Med. (2003); 348:1967-1976; Marra et al., Science (2003); 300:1399-1404; and Rota et al., Science (2003); 300:1394-1399). The SARS-associated coronavirus genome appears to be closer to the murine, bovine, porcine, and human coronaviruses in Group II and avian coronavirus IBV in Group I (Marra et al.,. Science 300:1399-404 (2003)).

At present, no effective therapy is available for the treatment of SARS.

Boron-Containing Compounds

Boric acid and various boronic acids have been used as inhibitors of β-lactamases (Koehler et al., Biochemistry 10, 2477-2483 (1971); Kiener et al., Biochem. J., 169, 197-204 (1978) (boric acid, phenylboronic acid and m-aminophenylboronate); Beesley et al., Biochem. J., 209, 229-233 (1983) (twelve substituted phenylborinic acids, including 2-formylphenylboronate, 4-formylphenylboronate, and 4-methylphenylboronate; and Amicosante et al., J. Chemotherapy, 1, 394-398 (1989) (boric acid, phenylboronic acid, m-aminophenylboronate and tetraphenylboronic acid)). m-(Dansylamidophenyl)-boronic acid has also been reported to be a submicromolar inhibitor of the Enterobacter cloacae P99 β-lactamase (Dryjanski et al., Biochemistry, 34, 3561-3568 (1995)). In addition, Strynadka and colleagues used the crystallographic structure of a mutant TEM-1 enzyme-penicillin G complex to design a novel alkylboronic acid inhibitor [(1R)-1-acetamido-2-(3-carboxyphenyl)ethane boronic acid] with high affinity for this enzyme. (Strynadka et al., Nat. Struc. Biol., 3, 688-695 (1996)). Various other boronic-acids are known and used as β-lactamase inhibitors. (e.g., Tondi et al., Chemistry & Biology, 8, 593-610 (2001); Martin et al., Bioorganic & Medicinal Chemistry Letters, 4(10), 1229-1234 (1994); Weston et al., J. Med. Chem., 41, 4577-4586 (1998); U.S. Pat. Nos. 6,075,014 and 6,184,363; and U.S. Provisional Patent Application Ser. No. 60/477,636, filed Jun. 10, 2003, and co-pending U.S. patent application Ser. No. 10/866,179, filed Jun. 10, 2004, both entitled “Beta-Lactamase Inhibitors and Methods of Use Thereof”.

No boron-containing compounds have been reported as inhibitors of coronavirus protease inhibitors.

Citation or identification of any references in the “Background of the Invention” or anywhere in the specification of this application is not an admission that such references available as prior art to the present invention.

### SUMMARY OF THE INVENTION

The present invention relates to boron-containing compounds.

In one particular embodiment, such compounds are boric acid and boronic acids.

In another particular embodiment, such compounds are organic boron-containing compounds.

In a first preferred embodiment, the compounds are described by formula (1):

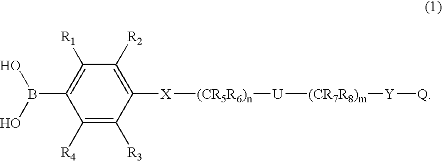

In a second preferred embodiment, the compounds are described by formula (2):

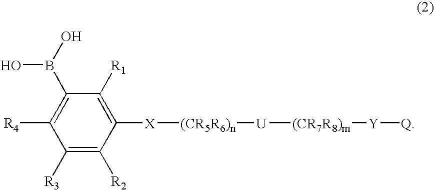

In a third preferred embodiment, the compounds are described by formula (3):

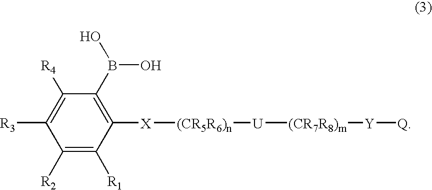

In a fourth preferred embodiment, the compounds are described by formula (4):

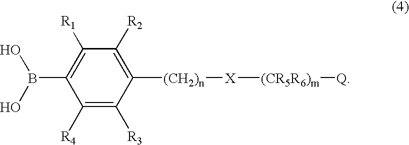

In a fifth preferred embodiment, the compounds are described by formula (5):

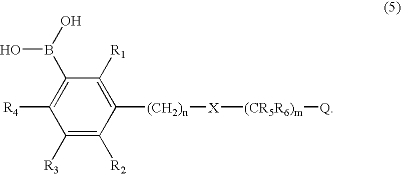

In a sixth preferred embodiment, the compounds are described by formula (6):

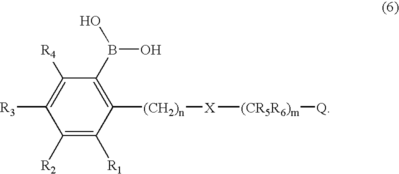

In a seventh preferred embodiment, the compounds are described by formula (7):

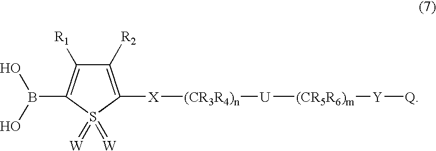

In an eighth preferred embodiment, the compounds are described by formula (8):

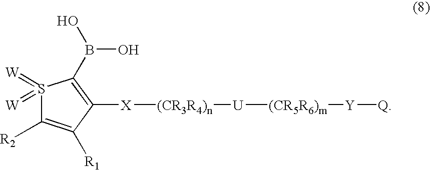

In a ninth preferred embodiment, the compounds are described by formula (9):

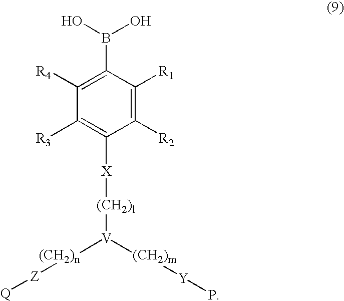

In a tenth preferred embodiment, the compounds are described by formula (10):

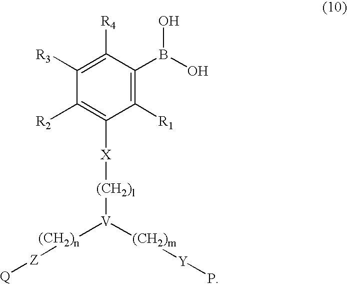

In an eleventh preferred embodiment, the compounds are described by formula (11):

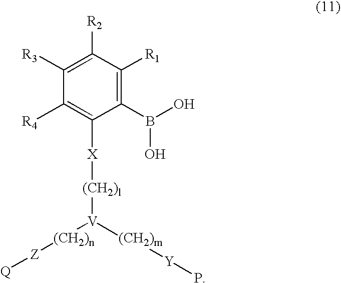

In a twelfth preferred embodiment, the compounds are described by formula (12):

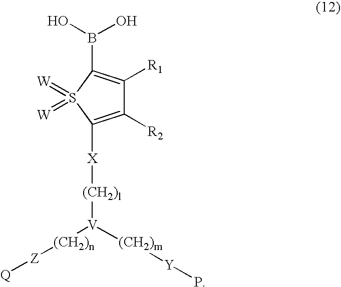

In a thirteenth preferred embodiment, the compounds are described by formula (13):

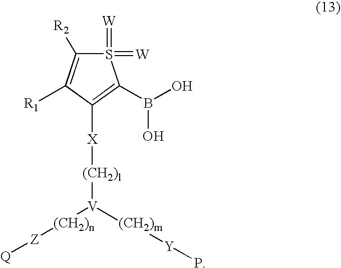

In a fourteenth preferred embodiment, the compounds are described by formula (14):

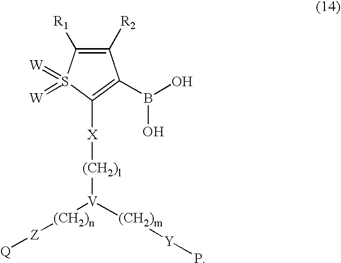

In a fifteenth preferred embodiment, the compounds are described by formula (15):

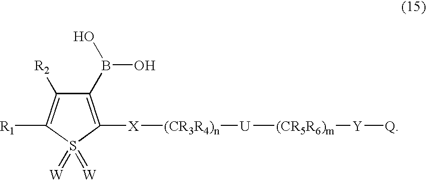

R₁ through R₈, l, m, n, P, Q. U, V, W, X, Y, and Z in formulae (1)-(15) can vary as set forth below, in order to optimize affinity, activity, absorption, distribution, metabolism, excretion, pharmacokinetic, toxicological and other properties required for their use as orally deliverable pharmaceuticals.

The invention also provides a method of inhibiting coronavirus protease(s), particularly coronavirus protease(s) that has one or more serine or threonine residue(s) at or near its active site, more particularly protease of SARS-associated coronavirus. The method comprises contacting the protease(s) with an effective amount of one or more boron-containing compounds, particularly compounds of formulae (1)-(15).

The invention additionally provides a method of treating infections caused by coronavirus, particularly by coronavirus that has protease(s) with one or more serine or threonine residue(s) at or near the protease active site, more particularly by SARS-associated coronavirus. Such method comprises administering to a subject suffering from such infections an effective amount of one or more boron-containing compounds, particularly compounds of formulae (1)-(15).

The invention further provides a method of detecting coronavirus, particularly coronavirus that has protease(s) with one or more serine or threonine residue(s) at or near the protease active site, more particularly by SARS-associated coronavirus. Such method comprises contacting the testing sample obtained from a patient with boron-containing compounds of formulae (1)-(15) that have been (1) tethered to an appropriate surface such that protease that becomes in contact and bound to the tethered compound can be detected; (2) labeled by fluorescent, radioactive or other markers that allow identification of coronavirus protease bound to the compound; or (3) that by any other mean can be used to detect the presence of coronavirus protease.

The invention also provides a pharmaceutical composition comprising one or more boron-containing compounds, particularly compounds of formulae (1)-(15), and a pharmaceutically acceptable carrier.

### BRIEF DESCRIPTION OF THE DRAWINGS

FIG. 1. shows a sequence alignment of identified SARS-associated coronavirus protease and the MHV protease. Identities are shown in dark grey and similarities in light grey.

FIG. 2. shows an alignment of sequences around the serine cluster for sequences identified by BLAST search with SARS-associated coronavirus protease 3CL^{pro}.

### DETAILED DESCRIPTION

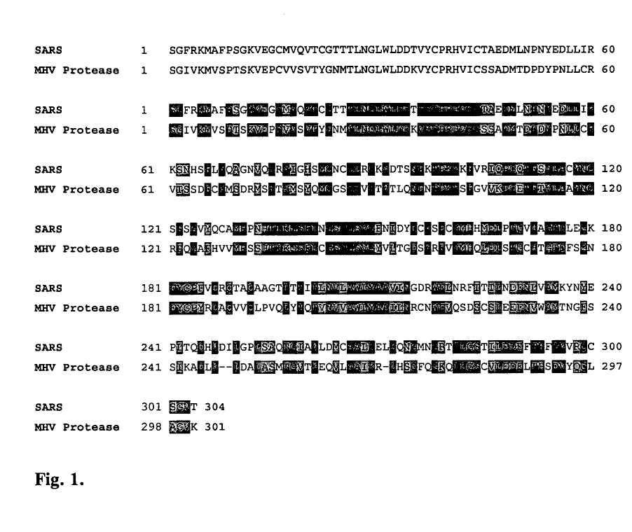

Analysis of the SARS-associated coronavirus genome identified the coding region for an essential protease (3CL^{pro}) homologous to that of other coronaviruses. The 3CL^{pro} coding is shown in FIG. 1 and in SEQ ID NO: 1. The highest homology was found with the mouse hepatitis coronavirus picorna 3C-like endopeptidase [MER02029] (MHV Protease) [SEQ ID NO: 2]. The alignment of the two sequences is shown in FIG. 1.

The sequences of the two enzymes are 50% identical (dark grey in FIG. 1) and 72% similar (dark grey and light grey in FIG. 1). These proteases are characterized by a catalytic cysteine (Cys 145) and histidine (His 41) and therefore are classified as cysteine proteases since the nucleophilic catalytic residue is a cysteine. These cysteine proteases are essential to the viral reproductive cycle since they are involved in the processing of all downstream domains of the replicase polyproteins of these viruses (Ziebuhr et al., (2000). Virus-encoded proteinases and proteolytic processing in the Nidovirales. J. Gen. Virol. 81, 853-879). For this reason, inhibition of this enzyme has been shown to inhibit viral replication in mouse hepatitis virus (MHV) infected cells (Kim et al., (1995). Coronavirus protein processing and RNA synthesis is inhibited by the cysteine proteinase inhibitor E64d. Virology 208, 1-8). The cleavage sites for the 3C-like viral proteases that have been studied are highly conserved, the P1 site being exclusively occupied by Gln and the P1′ site by small aliphatic residues (Ser, Ala, Asn, Gly, Cys).

The crystallographic structure of the SARS-associated coronavirus CL3^{pro} protease is available in the public protein database (accession code 1q2w). Analysis of the active site of the protease reveals a cluster of serines (Ser 139, Ser 144 and Ser 147).

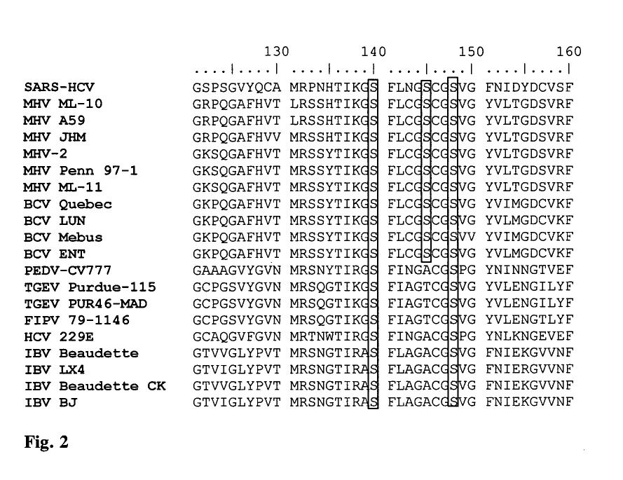

The serine cluster in the SARS-associated coronavirus protease 3CL^{pro} is highly conserved in similar proteins from other coronavirus indicating that either the same compound or similar compounds can be used to target this region of the binding site and inhibit these proteases. In addition, the entire region is highly conserved opening the possibility for wide spectrum antivirals targeting this region of the protease. FIG. 2 shows the sequence alignments between residues 121 and 160 for the proteases of twenty different coronaviruses. Conserved serine residues being targeted are boxed. The sequences shown are from the following Genbank accession numbers for protein sequences: SARS-HCV (severe acute respiratory syndrome-human coronavirus): NP_{—}828863 [SEQ ID NO: 3]; MHV ML-10 (murine hepatitis virus strain ML-10): AAF69341 [SEQ ID NO: 4]; MHV A59 (murine hepatitis virus strain A59): NP_{—}740610 [SEQ ID NO: 5]; MHV JHM (murine hepatitis virus strain JHM): P19751 [SEQ ID NO: 6]; MHV-2 (murine hepatitis virus strain 2): AAF19383 [SEQ ID NO: 7]; MHV Penn 97-1 (murine hepatitis virus strain Penn 97-1): AAF69331 [SEQ ID NO: 8]; MHV ML-11 (murine hepatitis virus strain ML-11): AAF68919 [SEQ ID NO: 9]; BCV Quebec (bovine coronavirus strain Quebec): AAL40396 [SEQ ID NO: 10]; BCV LUN (bovine coronavirus strain LUN): AAL57315 [SEQ ID NO: 11]; BCV Mebus (bovine coronavirus strain Mebus): AAA64744 [SEQ ID NO: 12]; BCV ENT (bovine coronavirus strain ENT): NP_{—}742132 [SEQ ID NO: 13]; PEDV-CV777 (porcine epidemic diarrhea virus strain CV777): NP_{—}839959 [SEQ ID NO: 14]; TGEV Purdue-115 (transmissible gastroenteritis virus strain Purdue-115): CAA83979 [SEQ ID NO: 15]; TGEV PUR₄₆-MAD (transmissible gastroenteritis virus strain PUR₄₆-MAD): NP_{—}840003 [SEQ ID NO: 16]; FIPV 79-1146 (feline infectious peritonitis virus strain 79-1146): AAK09095 [SEQ ID NO: 17]; HCV 229E (human coronavirus strain 229E): NP_{—}835346 [SEQ ID NO: 18]; IBV Beaudette (avian infectious bronchitis virus strain Beaudette): NP_{—}740623 [SEQ ID NO: 19]; IBV LX4 (avian infectious bronchitis virus strain LX4): AAQ21584 [SEQ ID NO: 20]; IBV Beaudette CK (avian infectious bronchitis virus strain Beaudette CK): CAC39112 [SEQ ID NO: 21]; IBV BJ (avian infectious bronchitis virus strain BJ): AAP92674 [SEQ ID NO: 22].

Without being bound by any particular theory, serine or threonine, particularly serine cluster, threonine cluster, and serine/threonine cluster, are chosen as a prime target site because the OH groups in serine and threonine residues are highly reactive with boron-containing compounds, particularly boric acid and boronic acids. Moreover, without being bound by any particular theory, since the coronavirus proteases such as SARS-associated coronavirus protease 3CL^{pro} contains a cluster of two or more serines and/or threonines (SARS-associated coronavirus protease contains a cluster of three serines), it is believed that multifunctional boron-containing compounds (i.e., a compound containing two or more boron atoms), particularly multifunctional boronic acids (i.e., a compound containing two or more —B(OH)₂ groups) would be even more potent, selective, and hence more effective than the monofunctional boron-containing compounds (i.e., a compound containing only one boron atom).

The boron-containing compounds of the present invention can be organic compounds that contain boron. Non-limiting examples of such boron-containing compounds include arylboronic acid, arylborates, arylboranes, alkylboronic acids, alkyl borates, alkylboranes and boron heterocyclics, and boron-containing compounds disclosed in Koehler et al., Biochemistry 10, 2477-2483 (1971); Kiener et al., Biochem. J., 169, 197-204 (1978); Beesley et al., Biochem. J., 209, 229-233 (1983); Amicosante et al., J. Chemotherapy, 1, 394-398 (1989); Dryjanski et al., Biochemistry, 34, 3561-3568 (1995); Strynadka et al., Nat. Struc. Biol., 3, 688-695 (1996); Tondi et al., Chemistry & Biology, 8, 593-610 (2001); Martin et al., Bioorganic & Medicinal Chemistry Letters, 4(10), 1229-1234 (1994); Weston et al., J. Med. Chem., 41, 4577-4586 (1998); U.S. Pat. Nos. 6,075,014 and 6,184,363; and co-pending U.S. Provisional Patent Application Ser. No. 60/477,636, entitled “Beta-Lactamase Inhibitors and Methods of Use Thereof,” filed: Jun. 10, 2003.

In one particular embodiment, the compound of the present invention is a compound described by formula (A) 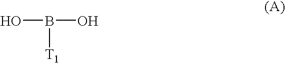
wherein T₁ comprises a ring structure or any other organic functional group; and
B is boron.

Non-limiting examples of T₁ include cycloalkyl, cyclic alkene or heterocyclic alkene with one or more substituents R.

In another particular embodiment, the compound of the present invention is a compound described by formula (B) 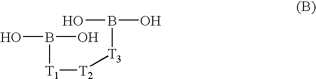
wherein T₁ and T₃ each comprises a ring structure or any other organic functional group; and T₂ is a linker; and
B is boron.

Non-limiting examples of T₁ and T₃ include cycloalkyl, cyclic alkene or heterocyclic alkene with one or more substituents R.

Non-limiting examples of the linker T₂ include di-branched linker such as —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR—, any di-branched cycloalkane, cyclic alkene or heterocyclic alkene with one or more substituents R, —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N— etc; each R independently represents any group, non-limiting examples include hydrogen, C₁₋₆ alkyl, C₃₋₇ cycloalkyl, C₁₋₆ alkoxy (e.g., n-butoxy, i-butoxy, sec-butoxy) trifluoromethyl, trifluoromethoxy, halogen, cyano, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl C₁₋₆ alkylcarbonyl, —CH═NOH, —CH₂NHOH, —C(CH₃)═NOH, —C(OH)═NOH, —SO₃H, —SO₂CH₃ and —SO₂CH₃ etc.

Non-limiting examples of cyclic alkene include, benzene, naphthalene, cyclopentene, cyclohexene, cyclopentadiene, cyclohexadiene, indene, fluorene, anthracene and phenanthrene.

Non-limiting examples of heterocyclic alkene include, furan, thiophene, pyrrole, pyrazole, imidazole, thiazole, oxazole, triazole, pyridine, pyran, thiopyran, pyridazine, pyrimidine, pyrazine, benzofuran, thionaphtene, indole, dibenzofuran, dibenzothiophene, carbazole, benzimidazole, indazole, benzoxadiazole, benzothiazole, coumarin, quinoline, isoquinoline, acridine, phenothiazine and phenazine.

In another particular embodiment, the compound of the present invention is a multifunctional boron-containing compound (i.e., a compound containing two or more boron atoms).

In another particular embodiment, the compound of the present invention is a multifunctional boronic acid (i.e., a compound containing two or more —B(OH)₂ groups).

This application includes Tables 1-15 which set forth without limitation representative compounds 1-403. In all cases, when a compound no. is listed in more than row of tables 1-15, it is to be understood that all of the substituents listed for a given compound no. are found together on a single compound. In Table 1, for example, compound no. 1 comprises the groups R₁=H, R₂=H, R₃=—NO₂, R₄=H, R₇=H, R₈=H, —X—=—CO—O—, —Y—=—O—CO—, m=0, n=0, and 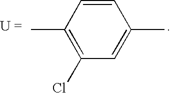
All other compounds listed in Tables 1-15 are to be similarly construed.

### 1. First Preferred Embodiment

In a first preferred embodiment, the compounds are described by formula (1): 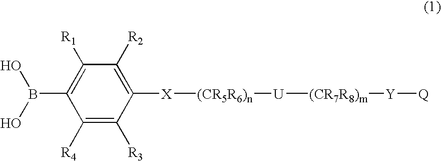
wherein R₁ through R₈, m, n, Q, U, X, and Y can vary in order to optimize affinity, activity, absorption, distribution, metabolism, excretion, pharmacokinetic, toxicological and other properties required for their use as orally deliverable pharmaceuticals.

### a. Preferred Embodiment 1-a

In a more preferred first embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ through R₈ each independently represents hydrogen, C₁₋₆ alkyl, C₃₋₇ cycloalkyl, benzyl or the carbon and attached two Rᵢs, they together form C₃₋₇ cycloalkyl;
m and n are each independently 0, 1, or 2;
X and Y each independently represents —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ⱼOR₁₈— (wherein R₁₈ is hydrogen or C₁₋₁ alkyl, and j is 1, 2 or 3), —CONR₁₉OH or —CHR₂₀N(COR₂₁)OH (wherein R₁₉ through R₂₁ each independently represents a hydrogen, C₁₋₁ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 1-b

In another more preferred embodiment of the first embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
R₅ through R₈ each independently represents hydrogen, C₁₋₆ alkyl, C₃₋₇ cycloalkyl, benzyl, or the carbon and attached two Rᵢs, they together form C₃₋₇ cycloalkyl;
m and n are each independently 0 or 1;
X and Y are each independently —CO—O—, —O—CO—, —CO—NH—, —NH—CO—, —CH═CH—, —NH—CH₂—, —CH₂—NH—, —SO₂—O—, —O—SO₂—, —SO₂—NH—, —NH—SO₂— or —N═N—;
U represents —(CH₂)ᵢ or —(CH═CH)ⱼ (wherein i=0, 1, 2, 3 or 4 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

### c. Preferred Embodiment 1-c

In another more preferred first embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ through R₈ each independently represents hydrogen, C₁₋₆ alkyl, C₃₋₇ cycloalkyl, benzyl or the carbon and attached two Rᵢs, they together form C₃₋₇ cycloalkyl;
m and n are each independently 0, 1, or 2;
X and Y each independently represents —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ⱼOR₁₈— (wherein R₁₈ is hydrogen or C₁₋₆ alkyl, and j is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
with the proviso that,
(1) when R₁ through R₄ are H, X is not a —NH—CH₂— or —CH₂—NH—;
(2) when R₁=R₂=H, X is not —CO—NH—;
(3) when Q is 3-boronophenyl, Y is not —CO—NH or —SO₂—NH—;
(4) when X=—CO—O—, Y=—O—CO—, m=0, n=0 and U is a 1,4-benzene, a 1,4-benzocyclic alkene or a 1,4-benzoheterocyclic alkene, Q is not 4-boronophenyl, which may be substituted with one or more substituents R₁₃;
(5) when R₁ through R₈ are H, X=—CO—O—, m=n=1, U=1,4-benzene, Y=—O—CO—, Q is not 4-boronophenyl;
(6) when R₁ through R₈ are H, X=—CO—O—, m=n=1, U=1,3-benzene, Y=—O—CO—, Q is not 4-boronophenyl;
(7) when R₁ through R₈ are H, X=—CO—O—, m=n=1, U=1,2-benzene, Y=—O—CO—, Q is not 4-boronophenyl;
(8) when R₁ through R₄ are H, X=—NH—SO₂—, m=n=0, U=4-methoxy-1,3-benzene, Y=—N═N—, Q is not 4-(dimethylamino)-1-naphthalenyl; and
(9) when R₁ is amino, R₂ through R₄ are H, X=Y=—N═N—, m=n=0, U is 1,4-naphthalene, Q is not 3-boronophenyl.

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### d. Preferred Embodiment 1-d

In another more preferred embodiment of the first embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₄ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
R₅ through R₈ each independently represents hydrogen, C₁₋₆ alkyl, C₃₋₇ cycloalkyl, benzyl, or the carbon and attached two Rᵢs, they together form C₃₋₇ cycloalkyl;
m and n are each independently 0 or 1;
X and Y are each independently —CO—O—, —O—CO—, —CO—NH—, —NH—CO—, —CH═CH—, —NH—CH₂—, —CH₂—NH—, —SO₂—O—, —O—SO₂—, —SO₂—NH—, —NH—SO₂— or —N═N—;
U represents —(CH₂)ᵢ or —(CH═CH)ⱼ (wherein i=0, 1, 2, 3 or 4 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
with the proviso that,
(1) when R₁ through R₄ are H, X is not a —NH—CH₂— or —CH₂—NH—;
(2) when R₁=R₂=H, X is not —CO—NH—;
(3) when Q is 3-boronophenyl, Y is not —CO—NH or —SO₂—NH—;
(4) when X=—CO—O—, Y=—O—CO—, m=0, n=0 and U is a 1,4-benzene, a 1,4-benzocyclic alkene or a 1,4-benzoheterocyclic alkene, Q is not 4-boronophenyl, which may be substituted with one or more substituents R₁₃;
(5) when R₁ through R₈ are H, X=—CO—O—, m=n=1, U=1,4-benzene, Y=—O—CO—, Q is not 4-boronophenyl;
(6) when R₁ through R₈ are H, X=—CO—O—, m=n=1, U=1,3-benzene, Y=—O—CO—, Q is not 4-boronophenyl;
(7) when R₁ through R₈ are H, X=—CO—O—, m=n=1, U=1,2-benzene, Y=—O—CO—, Q is not 4-boronophenyl;
(8) when R₁ through R₄ are H, X=—NH—SO₂—, m=n=0, U=4-methoxy-1,3-benzene, Y=—N═N—, Q is not 4-(dimethylamino)-1-naphthalenyl; and
(9) when R₁ is amino, R₂ through R₄ are H, X=Y=—N═N—, m=n=0, U is 1,4-naphthalene, Q is not 3-boronophenyl.

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (1) are set forth in Table 1 below.

TABLE 1
Representative Compounds in Formula (1):

| Compound | | | | | | | | | | | | |
|---|---|---|---|---|---|---|---|---|---|---|---|---|
| No. | R₁ | R₂ | R₃ | R₄ | R₅ | R₆ | R₇ | R₈ | —X— | —Y— | m | n |
| 1. | H | H | —NO₂ | H | | | H | H | —CO—O— | —O—CO— | 0 | 0 |
| 2. | H | H | H | H | | | | | —CO—O— | —CO—O— | 0 | 0 |
| 3. | —NO₂ | H | H | H | | | | | —CO—O— | —O—CO— | 0 | 0 |
| 4. | H | H | H | H | | | | | —CO—O— | —O—CO— | 0 | 0 |
| 5. | H | H | H | H | | | | | —CO—O— | —O—CO— | 0 | 0 |
| 6. | —NO₂ | H | H | H | | | | | —CO—O— | —O—CO— | 0 | 0 |
| 7. | H | H | H | H | | | | | —OCH₂— | —CH₂O— | 0 | 0 |
| 8. | H | H | H | H | | | | | —OCH₂— | —CH₂O— | 0 | 0 |
| 9. | H | F | H | H | | | | | —NHCH₂— | —CH₂NH— | 0 | 0 |
| 10. | H | H | H | H | H | H | | | —CO—O— | —CO—O— | 0 | 1 |
| 11. | H | H | H | H | H | H | H | H | —CO—O— | —CO—O— | 1 | 1 |
| 12. | H | H | H | H | H | H | H | H | —O—CO— | —CO—O— | 1 | 1 |
| 13. | H | H | H | H | H | H | H | H | —O—CO— | —CO—O— | 1 | 1 |
| 14. | H | H | H | H | H | H | H | H | —CO—O— | —O—CO— | 1 | 1 |
| 15. | H | H | H | H | H | H | H | H | —CO—O— | —SO₂—NH— | 1 | 1 |
| 16. | H | H | H | H | H | H | H | H | —CO—O— | —SO₂—NH— | 1 | 1 |
| 17. | H | H | H | H | | | | | —CO—NH— | —CO—NH— | 0 | 0 |
| 18. | H | H | H | H | | | | | —CO—NH— | —CO—O— | 0 | 0 |
| 19. | H | H | H | H | | | | | —CO—NH— | —CO—NH— | 0 | 0 |
| 20. | H | H | H | H | | | | | —CO—NH— | —CO—NH— | 0 | 0 |
| 21. | H | H | H | H | | | | | —CO—NH— | —CO—NH— | 0 | 0 |
| 22. | H | H | H | H | | | | | —CO—NH— | —CO—O— | 0 | 0 |
| 23. | H | H | H | H | | | | | —CO—NH— | —CO—O— | 0 | 0 |
| 24. | H | H | H | H | | | | | —CO—NH— | —CO—O— | 0 | 0 |
| 25. | H | H | H | H | | | | | —CO—NH— | —CO—O— | 0 | 0 |
| 26. | H | H | H | H | | | | | —CO—NH— | —CO—O— | 0 | 0 |
| 27. | H | H | H | H | | | | | —CO—NH— | —CO—O— | 0 | 0 |
| 28. | H | H | H | H | | | | | —CO—NH— | —CO—O— | 0 | 0 |
| 29. | H | H | H | H | | | | | —CO—NH— | —CO—O— | 0 | 0 |
| 30. | H | H | H | H | | | | | —CO—NH— | —CO—O— | 0 | 0 |
| 31. | H | H | H | H | | | | | —CO—NH— | —CO—NH— | 0 | 0 |
| 32. | H | H | H | H | | | | | —CO—NH— | —CO—NH— | 0 | 0 |
| 33. | H | H | H | H | | | | | —CO—NH— | —CO—NH— | 0 | 0 |
| 34. | H | H | H | H | | | | | —CO—NH— | —CO—NH— | 0 | 0 |
| 35. | H | H | H | H | | | | | —CO—NH— | —CO—NH— | 0 | 0 |
| 36. | H | H | H | H | | | | | —CO—NH— | —CO—NH— | 0 | 0 |

| Compound | |
|---|---|
| No. | —U— |
| 1. | 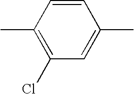 |
| 2. | 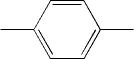 |
| 3. | 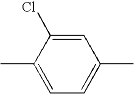 |
| 4. | 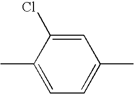 |
| 5. | 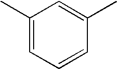 |
| 6. | 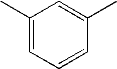 |
| 7. | 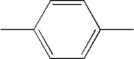 |
| 8. | 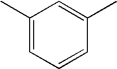 |
| 9. | 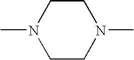 |
| 10. | 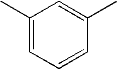 |
| 11. | 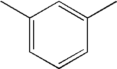 |
| 12. | 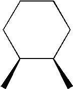 |
| 13. | 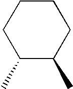 |
| 14. | 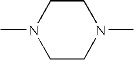 |
| 15. | 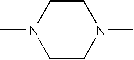 |
| 16. | 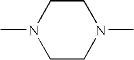 |
| 17. | —CH₂— |
| 18. | —CH₂— |
| 19. | 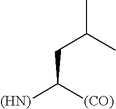 |
| 20. | 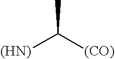 |
| 21. | 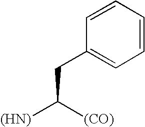 |
| 22. | 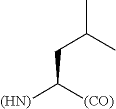 |
| 23. | 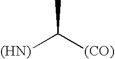 |
| 24. | 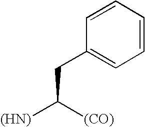 |
| 25. | 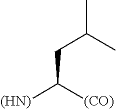 |
| 26. | 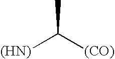 |
| 27. | 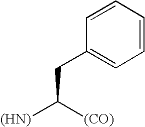 |
| 28. | 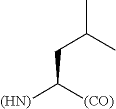 |
| 29. | 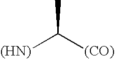 |
| 30. | 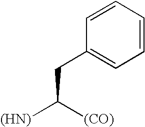 |
| 31. | 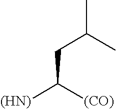 |
| 32. | 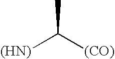 |
| 33. | 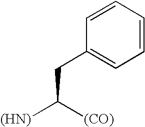 |
| 34. | 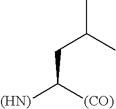 |
| 35. | 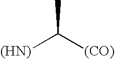 |
| 36. | 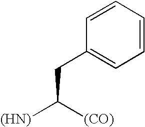 |

A most preferred compounds in Formula (1) is

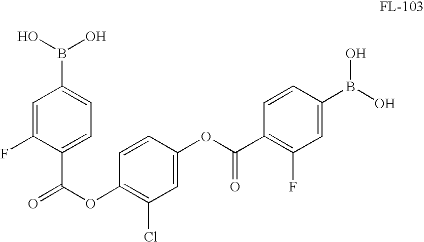

Additional compounds in Formula (1) are:

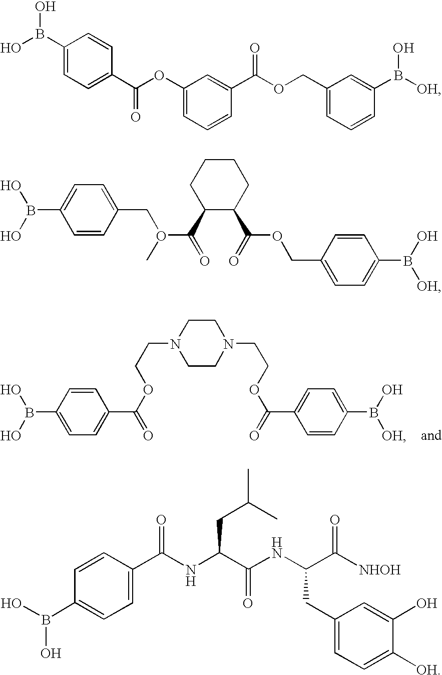

### 2. Second Preferred Embodiment

In a second preferred embodiment, the compounds are described by formula (2): 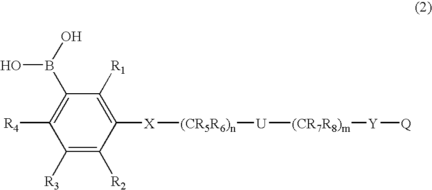
wherein R₁ through R₈, m, n, Q, U, X, and Y can vary in order to optimize affinity, activity, absorption, distribution, metabolism, excretion, pharmacokinetic, toxicological and other properties required for their use as orally deliverable pharmaceuticals.

### a. Preferred Embodiment 2-a

In a more preferred second embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ through R₈ each independently represents hydrogen, C₁₋₆ alkyl, C₃₋₇ cycloalkyl, benzyl or the carbon and attached two Rᵢs, they together form C₃₋₇ cycloalkyl;
m and n are each independently 0, 1, or 2;
X and Y each independently represents —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ⱼOR₁₈— (wherein R₁₈ is hydrogen or C₁₋₆ alkyl, and j is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₉ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 2-b

In another more preferred second embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, —SO₂NH₂;
R₅ through R₈ each independently represents hydrogen, C₁₋₆ alkyl, C₃₋₇ cycloalkyl, benzyl, or the carbon and attached two Rᵢs, they together form C₃₋₇ cycloalkyl;
m and n is each independently 0 or 1;
X and Y is each independently —CO—O—, —O—CO—, —CO—NH—, —NH—CO—, —CH═CH—, —NH—CH₂—, —CH₂—NH—, —SO₂—O—, —O—SO₂—, —SO₂—NH—, —NH—SO₂— or —N═N—;
U represents —(CH₂)ᵢ or —(CH═CH)ⱼ (wherein i=0, 1, or 2, or 3 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

### c. Preferred Embodiment 2-c

In a more preferred second embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ through R₈ each independently represents hydrogen, C₁₋₆ alkyl, C₃₋₇ cycloalkyl, benzyl or the carbon and attached two Rᵢs, they together form C₃₋₇ cycloalkyl;
m and n are each independently 0, 1, or 2;
X and Y each independently represents —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₁ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ⱼOR₁₈— (wherein R₁₈ is hydrogen or C₁₋₆ alkyl, and j is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
with the proviso that,
(1) when R₁ through R₄ represent hydrogen, X is not —NH—CO—, —NH—SO₂—, —NH—CH₂— or —CH₂—NH—;
(2) when Q is 3-boronophenyl, Y is not —CO—NH or —SO₂—NH—;
(3) when X=—CO—O—, Y=—O—CO—, m=0, n=0 and U is a 1,3-benzene, 1,4-benzene, a 1,3-benzocyclic alkene, a 1,4-benzocyclic alkene, a 1,3-benzoheterocyclic alkene or a 1,4-benzoheterocyclic alkene, Q is not 4-boronophenyl, which may be substituted with one or more substituents R₁₃;
(4) when R₄ is borono, R₁ through R₃ and R₅ through R₈ are H, X=—CO—NH—, Y=—CO—O—, m=n=1, U=—CH₂—CH₂—CH₂—, Q is not 2,5-dioxo-1-pyrrolidinyl; and
(5) when R₃ is borono, R₁, R₂ and R₅ through R₈ are H, X=—CO—NH—, Y=—CO—O—, m=n=1, U=—CH₂—CH₂—CH₂—, Q is not 2,5-dioxo-1-pyrrolidinyl.

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### d. Preferred Embodiment 2-d

In another more preferred second embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, —SO₂NH₂;
R₅ through R₈ each independently represents hydrogen, C₁₋₆ alkyl, C₃₋₇ cycloalkyl, benzyl, or the carbon and attached two Rᵢs, they together form C₃₋₇ cycloalkyl;
m and n is each independently 0 or 1;
X and Y is each independently —CO—O—, —O—CO—, —CO—NH—, —NH—CO—, —CH═CH—, —NH—CH₂—, —CH₂—NH—, —SO₂—O—, —O—SO₂—, —SO₂—NH—, —NH—SO₂— or —N═N—;
U represents —(CH₂)ᵢ or —(CH═CH)ⱼ (wherein i=0, 1, 2, or 3 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₆₋₄ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
with the proviso that,
(1) when R₁ through R₄ represent hydrogen, X is not —NH—CO—, —NH—SO₂—, —NH—CH₂— or —CH₂—NH—;
(2) when Q is 3-boronophenyl, Y is not —CO—NH or —SO₂—NH—;
(3) when X=—CO—O—, Y=—O—CO—, m=0, n=0 and U is a 1,3-benzene, 1,4-benzene, a 1,3-benzocyclic alkene, a 1,4-benzocyclic alkene, a 1,3-benzoheterocyclic alkene or a 1,4-benzoheterocyclic alkene, Q is not 4-boronophenyl, which may be substituted with one or more substituents R₁₃;
(4) when R₄ is borono, R₁ through R₃ and R₅ through R₈ are H, X=—CO—NH—, Y=—CO—O—, m=n=1, U=—CH₂—CH₂—CH₂—, Q is not 2,5-dioxo-1-pyrrolidinyl; and
(5) when R₃ is borono, R₁, R₂ and R₅ through R₈ are H, X=—CO—NH—, Y=—CO—O—, m=n=1, U=—CH₂—CH₂—CH₂—, Q is not 2,5-dioxo-1-pyrrolidinyl.

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, the heterocyclic alkene means a cyclic alkene as defined above, wherein one, two, or all three rings contain(s) one or more S, N or O atom(s).

Non-limiting representative compounds of formula (2) are set forth in Table 2 below.

TABLE 2
Representative Compounds in Formula (2):

| Compound | | | | | | | | | | | | |
|---|---|---|---|---|---|---|---|---|---|---|---|---|
| No. | R₁ | R₂ | R₃ | R₄ | R₅ | R₆ | R₇ | R₈ | —X— | —Y— | m | n |
| 37. | H | H | —NO₂ | H | | | | | —CO—O— | —O—CO— | 0 | 0 |
| 38. | H | H | —NO₂ | H | | | | | —CO—O— | —O—CO— | 0 | 0 |
| 39. | H | H | —NO₂ | H | H | H | H | H | —CO—O— | —O—CO— | 1 | 1 |
| 40. | H | H | —NO₂ | H | | | | | —CO—O— | —O—CO— | 0 | 0 |
| 41. | H | H | H | H | | | | | —O—CO— | —CO—O— | 0 | 0 |
| 42. | H | H | —NO₂ | H | | | | | —O—CO— | —CO—O— | 0 | 0 |
| 43. | H | H | —NO₂ | H | | | | | —O—CO— | —CO—O— | 0 | 0 |
| 44. | H | H | —H | H | | | | | —O—CO— | —CO—O— | 0 | 0 |
| 45. | H | H | —NO₂ | H | | | | | —CO—O— | —CO—NH— | 0 | 0 |
| 46. | H | H | —NO₂ | H | | | | | —CO—O— | —CO—NH— | 0 | 0 |
| 47. | H | H | H | H | | | | | —CO—O— | —CO—NH— | 0 | 0 |
| 48. | H | H | H | H | | | | | —CO—O— | —CO—NH— | 0 | 0 |
| 49. | H | H | —NO₂ | H | | | | | —CO—NH— | —CO—NH— | 0 | 0 |
| 50. | H | H | —NO₂ | H | | | | | —CO—NH— | —CO—O— | 0 | 0 |
| 51. | H | H | —NO₂ | H | | | | | —CO—NH— | —CO—NH— | 0 | 0 |
| 52. | H | H | —NO₂ | H | | | | | —CO—NH— | —CO—NH— | 0 | 0 |
| 53. | H | H | —NO₂ | H | | | | | —CO—NH— | —CO—NH— | 0 | 0 |
| 54. | H | H | —NO₂ | H | | | | | —CO—NH— | —CO—O— | 0 | 0 |
| 55. | H | H | —NO₂ | H | | | | | —CO—NH— | —CO—O— | 0 | 0 |
| 56. | H | H | —NO₂ | H | | | | | —CO—NH— | —CO—O— | 0 | 0 |
| 57. | H | H | —NO₂ | H | | | | | —CO—NH— | —CO—O— | 0 | 0 |
| 58. | H | H | —NO₂ | H | | | | | —CO—NH— | —CO—O— | 0 | 0 |
| 59. | H | H | —NO₂ | H | | | | | —CO—NH— | —CO—O— | 0 | 0 |
| 60. | H | H | —NO₂ | H | | | | | —CO—NH— | —CO—O— | 0 | 0 |
| 61. | H | H | —NO₂ | H | | | | | —CO—NH— | —CO—O— | 0 | 0 |
| 62. | H | H | —NO₂ | H | | | | | —CO—NH— | —CO—O— | 0 | 0 |
| 63. | H | H | H | H | | | | | —O—CO— | —CO—O— | 0 | 0 |
| 64. | H | H | H | H | | | | | —O—CO— | —CO—O— | 0 | 0 |
| 65. | H | H | H | H | | | | | —O—CO— | —CO—O— | 0 | 0 |
| 66. | H | H | H | H | | | | | —O—CO— | —CO—O— | 0 | 0 |
| 67. | H | H | H | H | H | H | H | H | —O—CO— | —CO—O— | 1 | 1 |
| 68. | H | H | H | H | H | H | H | H | —O—CO— | —CO—O— | 1 | 1 |
| 69. | H | H | —NO₂ | H | H | H | H | H | —CO—O— | —O—CO— | 1 | 1 |
| 70. | H | H | —NO₂ | H | H | H | H | H | —CO—O— | —SO2—NH— | 1 | 1 |
| 71. | H | H | —NO₂ | H | H | H | H | H | —CO—O— | —SO2—NH— | 1 | 1 |
| 72. | H | H | —N(CH₃)₂ | H | | | | | —CO—O— | —O—CO— | 0 | 0 |
| 73. | H | H | —NO₂ | H | | | | | —CO—NH— | —CO—NH— | 0 | 0 |
| 74. | H | H | —NO₂ | H | | | | | —CO—NH— | —CO—NH— | 0 | 0 |
| 75. | H | H | —NO₂ | H | | | | | —CO—NH— | —CO—NH— | 0 | 0 |
| 76. | H | H | —NO₂ | H | | | | | —CO—NH— | —CO—NH— | 0 | 0 |
| 77. | H | H | —NO₂ | H | | | | | —CO—NH— | —CO—NH— | 0 | 0 |
| 78. | H | H | —NO₂ | H | | | | | —CO—NH— | —CO—NH— | 0 | 0 |

| Compound | | |
|---|---|---|
| No. | —U— | —Q |
| 37. | 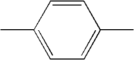 | 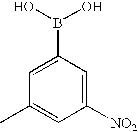 |
| 38. | 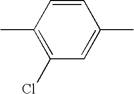 | 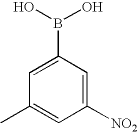 |
| 39. | 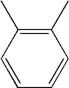 | 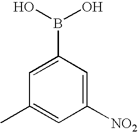 |
| 40. | 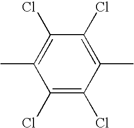 | 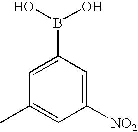 |
| 41. | 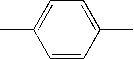 | 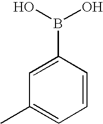 |
| 42. | 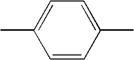 | 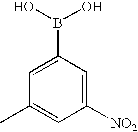 |
| 43. | 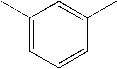 | 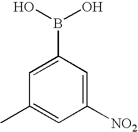 |
| 44. | 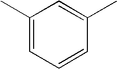 | 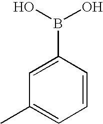 |
| 45. | 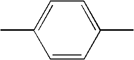 | 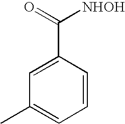 |
| 46. | 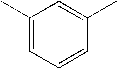 | 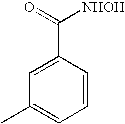 |
| 47. | 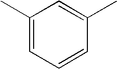 | 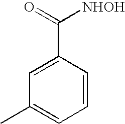 |
| 48. | 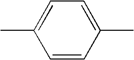 | 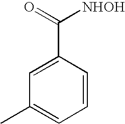 |
| 49. | —CH₂— | 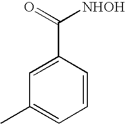 |
| 50. | —CH₂— | 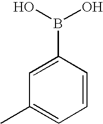 |
| 51. | 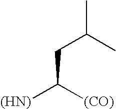 | 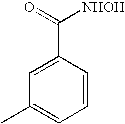 |
| 52. | 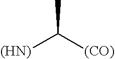 | 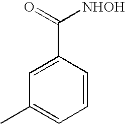 |
| 53. | 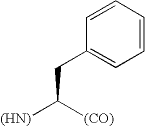 | 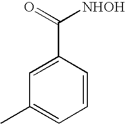 |
| 54. | 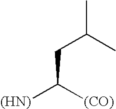 | 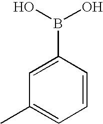 |
| 55. | 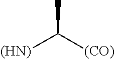 | 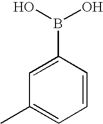 |
| 56. | 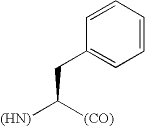 | 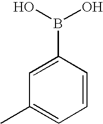 |
| 57. | 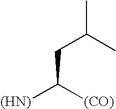 | 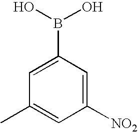 |
| 58. | 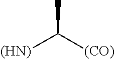 | 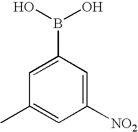 |
| 59. | 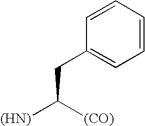 | 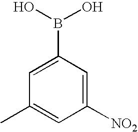 |
| 60. | 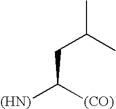 | 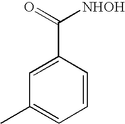 |
| 61. | 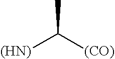 | 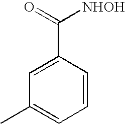 |
| 62. | 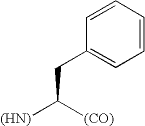 | 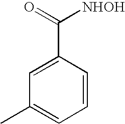 |
| 63. | 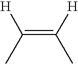 | 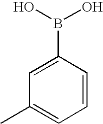 |
| 64. | 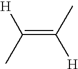 | 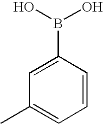 |
| 65. | 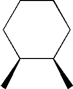 | 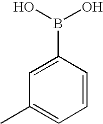 |
| 66. | 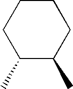 | 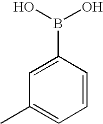 |
| 67. | 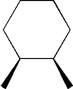 | 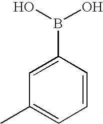 |
| 68. | 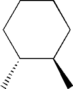 | 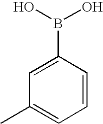 |
| 69. | 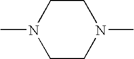 | 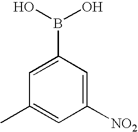 |
| 70. | 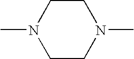 | 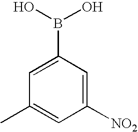 |
| 71. | 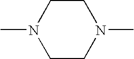 | 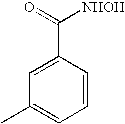 |
| 72. | 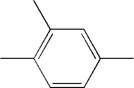 | 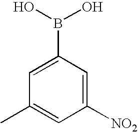 |
| 73. | 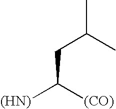 | 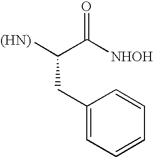 |
| 74. | 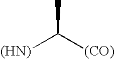 | 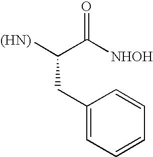 |
| 75. | 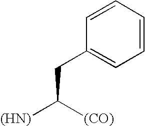 | 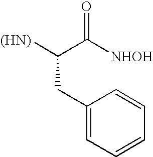 |
| 76. | 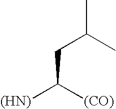 | 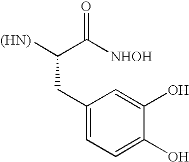 |
| 77. | 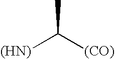 | 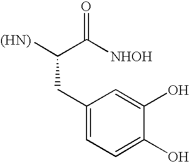 |
| 78. | 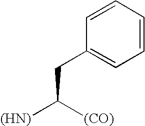 | 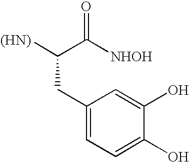 |

Most preferred compounds in Formula (2) are:

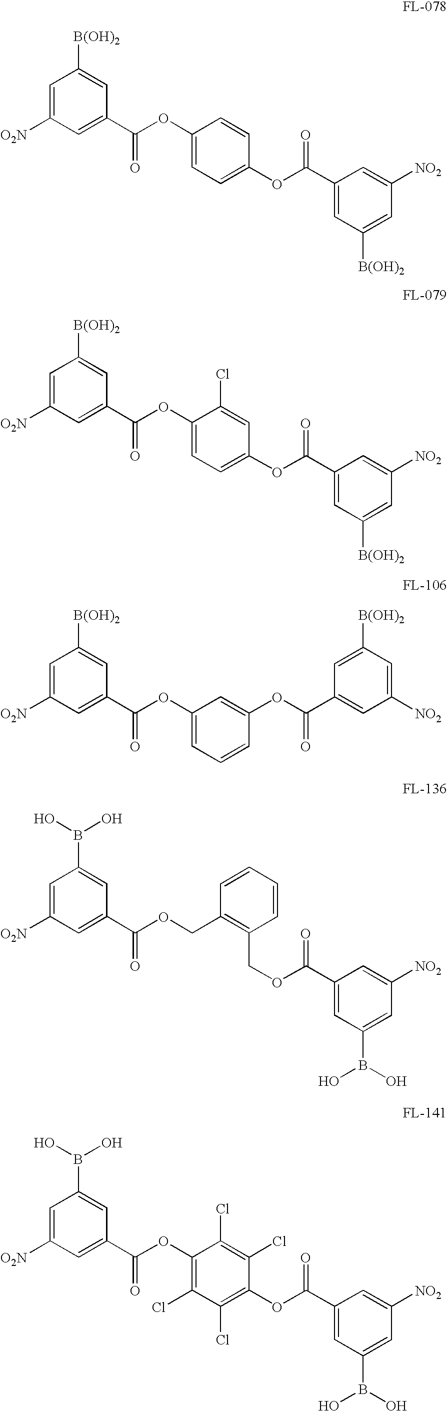

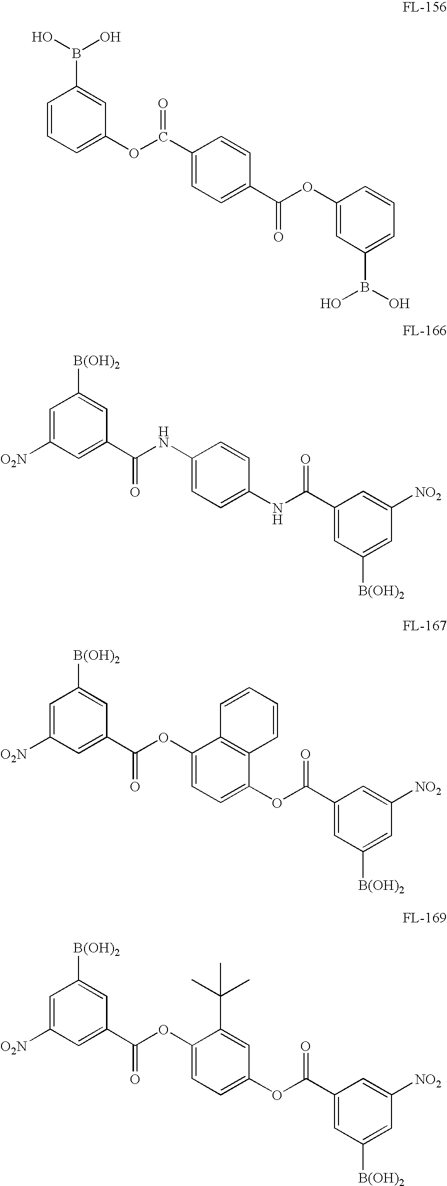

Additional compounds in Formula (2) are:

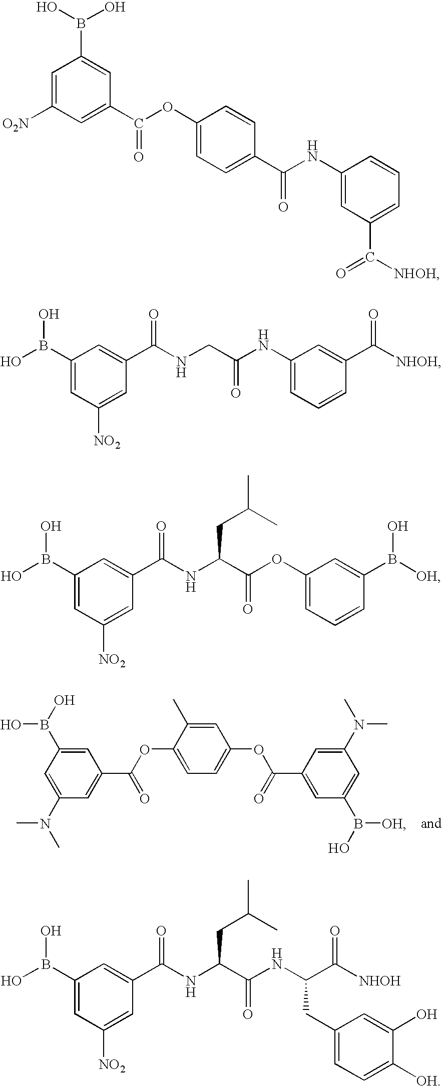

### 3. Third Preferred Embodiment

In a third preferred embodiment, the compounds are described by formula (3): 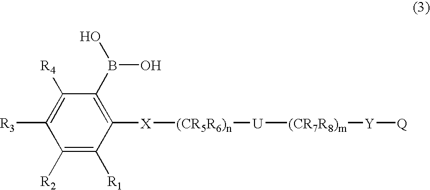
wherein R₁ through R₈, m, n, Q, U, X, and Y can vary in order to optimize affinity, activity, absorption, distribution, metabolism, excretion, pharmacokinetic, toxicological and other properties required for their use as orally deliverable pharmaceuticals.

### a. Preferred Embodiment 3-a

In a more preferred third embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ through R₈ each independently represents hydrogen, C₁₋₆ alkyl, C₃₋₇ cycloalkyl, benzyl or the carbon and attached two Rᵢs, they together form C₃₋₇ cycloalkyl;
m and n are each independently 0, 1, or 2;
X and Y each independently represents —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ⱼOR₁₈— (wherein R₁₈ is hydrogen or C₁₋₆ alkyl, and j is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₁ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 3-b

In another more preferred third embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, SO₂CH₃, —SO₂NH₂;
R₅ through R₈ each independently represents hydrogen, C₁₋₆ alkyl, C₃₋₇ cycloalkyl, benzyl, or the carbon and attached two Rᵢs, they together form C₃₋₇ cycloalkyl;
m and n is each independently 0 or 1;
X and Y is each independently —CO—O—, —O—CO—, —CO—NH—, —NH—CO—, —CH═CH—, —NH—CH₂—, —CH₂—NH—, —SO₂—O—, —O—SO₂—, —SO₂—NH— or —NH—SO₂—;
U represents —(CH₂)ᵢ or —(CH═CH)ⱼ (wherein i=0, 1, 2 or 3 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO— or —CH₂—).

In this particular embodiment, each heterocyclic alkene is independently a cyclic alkene as defined above, wherein one, two, or all three rings contain(s) one or more S, N or O atom(s).

### c. Preferred Embodiment 3-c

In another more preferred third embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉H or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ through R₈ each independently represents hydrogen, C₁₋₆ alkyl, C₃₋₇ cycloalkyl, benzyl or the carbon and attached two Rᵢs, they together form C₃₋₇ cycloalkyl;
m and n are each independently 0, 1, or 2;
X and Y each independently represents —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ⱼOR₁₈— (wherein R₁₈ is hydrogen or C₁₋₆ alkyl, and j is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
with the proviso that,
(1) when R₁ through R₄ are H, X is not —CH₂—NH—; and
(2) when Q is 3-boronophenyl, Y is not —CO—NH or —SO₂—NH—.

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### d. Preferred Embodiment 3-d

In another more preferred third embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, —SO₂NH₂;
R₅ through R₈ each independently represents hydrogen, C₁₋₆ alkyl, C₃₋₇ cycloalkyl, benzyl, or the carbon and attached two Rᵢs, they together form C₃₋₇ cycloalkyl;
m and n is each independently 0 or 1;
X and Y is each independently —CO—O—, —O—CO—, —CO—NH—, —NH—CO—, —CH═CH—, —NH—CH₂—, —CH₂—NH—, —SO₂—O—, —O—SO₂—, —SO₂—NH— or —NH—SO₂—;
U represents —(CH₂)ᵢ or —(CH═CH)ⱼ(wherein i=0, 1, 2 or 3 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
with the proviso that,
(1) when R₁ through R₄ are H, X is not —CH₂—NH—, and
(2) when Q is 3-boronophenyl, Y is not —CO—NH or —SO₂—NH—.

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO— or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (3) are set forth in Table 3 below.

TABLE 3
Representative Compounds in Formula (3):

| Compound | | | | | | | | | | | | |
|---|---|---|---|---|---|---|---|---|---|---|---|---|
| No. | R₁ | R₂ | R₃ | R₄ | R₅ | R₆ | R₇ | R₈ | —X— | —Y— | m | n |
| 79. | H | H | H | H | | | | | —CO—O— | —CO—O— | 0 | 0 |
| 80. | H | H | H | H | | | | | —CO—O— | —O—CO— | 0 | 0 |
| 81. | H | H | H | H | | | | | —CO—O— | —O—CO— | 0 | 0 |
| 82. | H | H | H | H | | | | | —OCH₂— | —CH₂O— | 0 | 0 |
| 83. | H | H | H | H | | | | | —OCH₂— | —CH₂O— | 0 | 0 |
| 84. | H | F | H | H | | | | | —NHCH₂— | —CH₂NH— | 0 | 0 |
| 85. | H | H | H | H | H | H | | | —CO—O— | —CO—O— | 0 | 1 |
| 86. | H | H | H | H | H | H | H | H | —CO—O— | —CO—O— | 1 | 1 |
| 87. | H | H | H | H | H | H | H | H | —O—CO— | —CO—O— | 1 | 1 |
| 88. | H | H | H | H | H | H | H | H | —O—CO— | —CO—O— | 1 | 1 |
| 89. | H | H | H | H | H | H | H | H | —CO—O— | —O—CO— | 1 | 1 |
| 90. | H | H | H | H | H | H | H | H | —CO—O— | —SO₂—NH— | 1 | 1 |
| 91. | H | H | H | H | H | H | H | H | —CO—O— | —SO₂—NH— | 1 | 1 |
| 92. | H | H | H | H | | | | | —CO—O— | —O—CO— | 0 | 0 |
| 93. | H | H | H | H | | | | | —CO—NH— | —CO—NH— | 0 | 0 |
| 94. | H | H | H | H | | | | | —CO—NH— | —CO—O— | 0 | 0 |
| 95. | H | H | H | H | | | | | —CO—NH— | —CO—NH— | 0 | 0 |
| 96. | H | H | H | H | | | | | —CO—NH— | —CO—NH— | 0 | 0 |
| 97. | H | H | H | H | | | | | —CO—NH— | —CO—NH— | 0 | 0 |
| 98. | H | H | H | H | | | | | —CO—NH— | —CO—O— | 0 | 0 |
| 99. | H | H | H | H | | | | | —CO—NH— | —CO—O— | 0 | 0 |
| 100. | H | H | H | H | | | | | —CO—NH— | —CO—O— | 0 | 0 |
| 101. | H | H | H | H | | | | | —CO—NH— | —CO—NH— | 0 | 0 |
| 102. | H | H | H | H | | | | | —CO—NH— | —CO—NH— | 0 | 0 |
| 103. | H | H | H | H | | | | | —CO—NH— | —CO—NH— | 0 | 0 |
| 104. | H | H | H | H | | | | | —CO—NH— | —CO—NH— | 0 | 0 |
| 105. | H | H | H | H | | | | | —CO—NH— | —CO—NH— | 0 | 0 |
| 106. | H | H | H | H | | | | | —CO—NH— | —CO—NH— | 0 | 0 |

| Compound | | |
|---|---|---|
| No. | —U— | —Q |
| 79. | 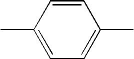 | 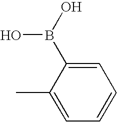 |
| 80. | 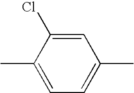 | 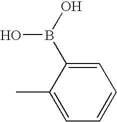 |
| 81. | 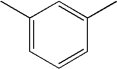 | 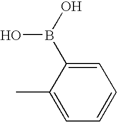 |
| 82. | 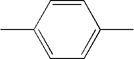 | 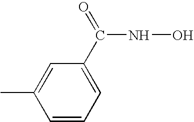 |
| 83. | 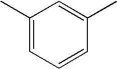 | 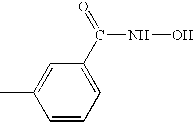 |
| 84. | 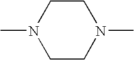 | 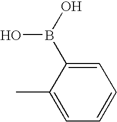 |
| 85. | 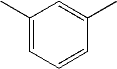 | 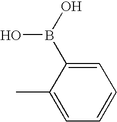 |
| 86. | 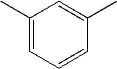 | 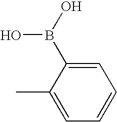 |
| 87. | 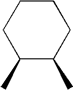 | 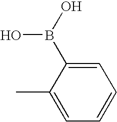 |
| 88. | 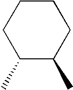 | 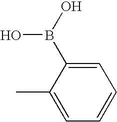 |
| 89. | 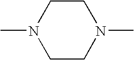 | 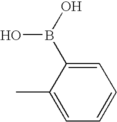 |
| 90. | 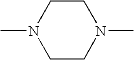 | 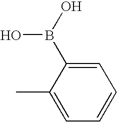 |
| 91. | 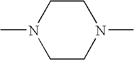 | 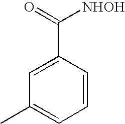 |
| 92. | 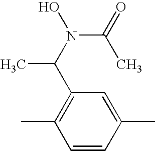 | 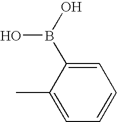 |
| 93. | —CH₂— | 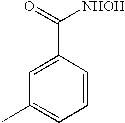 |
| 94. | —CH₂— | 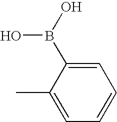 |
| 95. | 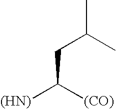 | 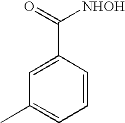 |
| 96. | 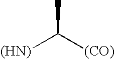 | 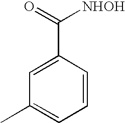 |
| 97. | 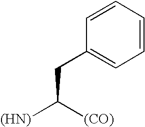 | 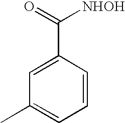 |
| 98. | 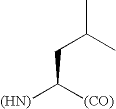 | 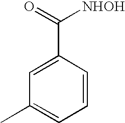 |
| 99. | 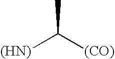 | 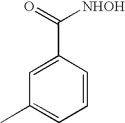 |
| 100. | 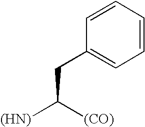 | 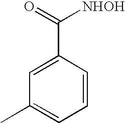 |
| 101. | 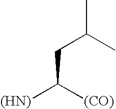 | 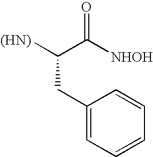 |
| 102. | 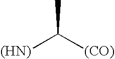 | 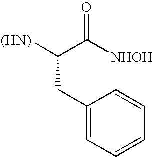 |
| 103. | 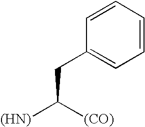 | 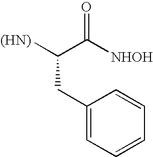 |
| 104. | 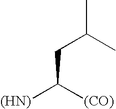 | 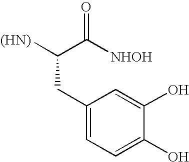 |
| 105. | 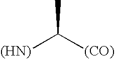 | 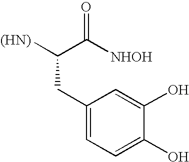 |
| 106. | 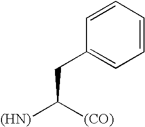 | 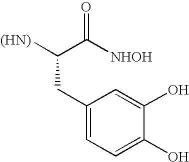 |

The most preferred compounds in Formula (3) are:

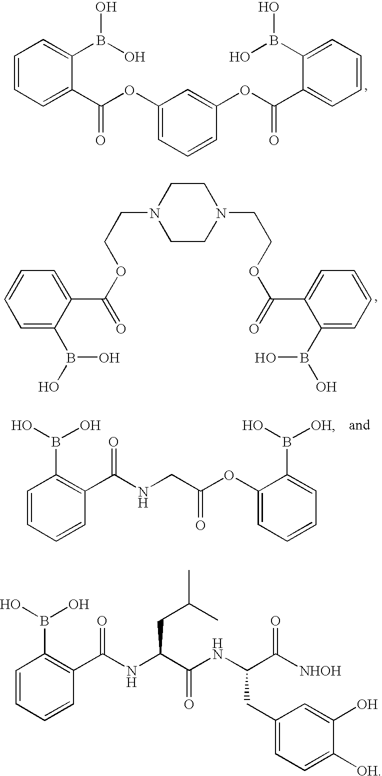

### 54. Fourth Preferred Embodiment

In a fourth preferred embodiment, the compounds are described by formula (4): 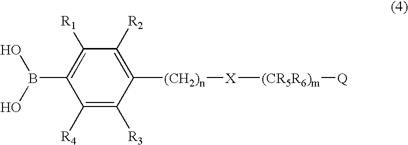
wherein R₁ through R₆, m, n, Q, and X can vary in order to optimize affinity, activity, absorption, distribution, metabolism, excretion, pharmacokinetic, toxicological and other properties required for their use as orally deliverable pharmaceuticals.

### a. Preferred Embodiment 4-a

In a more preferred fourth embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, formyl;
m and n is each independently 0, 1 or 2;
X is —O—, —NH—, —S—, —SO₂—, —CO—, —CH₂—, —CH═CH—CO—O—, —CH═CH—CO—NH—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 4-b

In another more preferred fourth embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₄ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0 or 1;
X is —CO—O—, —CH═CH—CO—O—, —O—CO—, —CO—NH—, —CH═CH—CO—NH—, —NH—CO—, —CH═N—, —CH═CH—, —NH—CH₂—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₁ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

### c. Preferred Embodiment 4-c

In another preferred fourth embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₁ alkyl, C₃₋₇ cycloalkyl, trifluoromethyl, phenyl or benzyl);
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, formyl;
m and n is each independently 0, 1 or 2;
X is —O—, —NH—, —S—, —SO₂—, —CO—, —CH₂—, —CH═CH—CO—O—, —CH═CH—CO—NH—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
with the proviso that,
(1) when m=0 and Q is 3-boronophenyl, X is not —CO—NH or —SO₂—NH—;
(2) when R₁ through R₄ are H, m=n=0, X is not —CO—O—;
(3) when R₁=R₂=H, X is not —CO—NH—;
(4) when X=—CO—O—, R₅=H, R₆=H, m=1 and n=0, Q is not a benzene, a benzocyclic alkene or a benzoheterocyclic alkene, which may be substituted with one or more substituents R₁₃;
(5) when X=—CO—O—, m=0 and n=0, Q is not a benzene, a benzocyclic alkene or a benzoheterocyclic alkene, which may be substituted with one or more substituents R₁₃;
(6) when R₁ through R₄ are H, X=—CO—O— and m=n=0, Q is not 2,5-dioxo-1-pyrrolidinyl;
(7) when R₁ through R₄ are H, X=—CO—O— and m=n=0, Q is not cyclohexyl;
(8) when R₁ through R₄ are H, X=—O—CO— and m=n=0, Q is not cyclopropyl;
(9) when R₁ through R₄ are H, X=—NH—SO₂—, m=n=0, Q is not 4-(4,5-dihydro-3-phenyl-1H-pyrazol-1-yl)phenyl;
(10) when R₁ through R₄ are H, X=—NH—SO₂—, m=n=0, Q is not 5-(dimethylamino)-1-naphthalenyl;
(11) when R₁ through R₄ are H, X=—NH—CH₂— and m=n=0, Q is not phenyl;
(12) when R₁ through R₄ are H, X=—NH—CO—, m=n=0, Q is not phenyl;
(13) when R₁ through R₄ are H, X=—NH—CO— and m=n=0, Q is not phenyl;
(14) when R₁ through R₄ are H, X=—NH—CO—, m=n=0, Q is not 4-chloro-3-(4-methyl-1-piperazinyl)phenyl;
(15) when R₁ through R₄ are H, X=—NH—CO—, m=n=0, Q is not 4-methoxy-3-(4-methyl-1-piperazinyl)phenyl;
(16) when R₁ through R₄ are H, X=—NH—CO—, m=n=0, Q is not 3-methoxy-4-(4-methyl-1-piperazinyl)phenyl;
(17) when R₁, R₃ through R₆ are H, R₂ is methoxyl, X=—NH—CO—, m=2, n=0, Q is not phenyl;
(18) when R₁ through R₄ are H, X=—N═N—, m=n=0, Q is not 4-(dimethylamino)phenyl;
(19) when R₁ is amino, R₂ through R₄ are H, X=—N═N—, m=n=0, Q is not 1-naphthalenyl;
(20) when R₁ is amino, R₂ through R₄ are H, X=—N═N—, m=n=0, Q is not 4-carboxyphenyl; and
(21) when R₁ through R₄ are H, X=—N═N—, m=n=0, Q is not 2-hydroxy-1-naphthalenyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### d. Preferred Embodiment 4-d

In another more preferred fourth embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0 or 1;
X is —CO—O—, —CH═CH—CO—O—, —O—CO—, —CO—NH—, —CH═CH—CO—NH—, —NH—CO—, —CH═N—, —CH═CH—, —NH—CH₂—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁, represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
with the proviso that,
(1) when m=0 and Q is 3-boronophenyl, X is not —CO—NH or —SO₂—NH—;
(2) when R₁ through R₄ are H, m=n=0, X is not —CO—O—;
(3) when R₁=R₂=H, X is not —CO—NH—;
(4) when X=—CO—O—, R₅=H, R₆=H, m=1 and n=0, Q is not a benzene, a benzocyclic alkene or a benzoheterocyclic alkene, which may be substituted with one or more substituents R₁₃;
(5) when X=—CO—O—, m=0 and n=0, Q is not a benzene, a benzocyclic alkene or a benzoheterocyclic alkene, which may be substituted with one or more substituents R₁₃;
(6) when R₁ through R₄ are H, X=—CO—O— and m=n=0, Q is not 2,5-dioxo-1-pyrrolidinyl;
(7) when R₁ through R₄ are H, X=—CO—O— and m=n=0, Q is not cyclohexyl;
(8) when R₁ through R₄ are H, X=—O—CO— and m=n=0, Q is not cyclopropyl;
(9) when R₁ through R₄ are H, X=—NH—SO₂—, m=n=0, Q is not 4-(4,5-dihydro-3-phenyl-1H-pyrazol-1-yl)phenyl;
(10) when R₁ through R₄ are H, X=—NH—SO₂—, m=n=0, Q is not 5-(dimethylamino)-1-naphthalenyl;
(11) when R₁ through R₄ are H, X=—NH—CH₂— and m=n=0, Q is not phenyl;
(12) when R₁ through R₄ are H, X=—NH—CO—, m=n=0, Q is not phenyl;
(13) when R₁ through R₄ are H, X=—NH—CO— and m=n=0, Q is not phenyl;
(14) when R₁ through R₄ are H, X=—NH—CO—, m=n=0, Q is not 4-chloro-3-(4-methyl-1-piperazinyl)phenyl;
(15) when R₁ through R₄ are H, X=—NH—CO—, m=n=0, Q is not 4-methoxy-3-(4-methyl-1-piperazinyl)phenyl;
(16) when R₁ through R₄ are H, X=—NH—CO—, m=n=0, Q is not 3-methoxy-4-(4-methyl-1-piperazinyl)phenyl;
(17) when R₁, R₃ through R₆ are H, R₂ is methoxyl, X=—NH—CO—, m=2, n=0, Q is not phenyl;
(18) when R₁ through R₄ are H, X=—N═N—, m=n=0, Q is not 4-(dimethylamino)phenyl;
(19) when R₁ is amino, R₂ through R₄ are H, X=—N═N—, m=n=0, Q is not 1-naphthalenyl;
(20) when R₁ is amino, R through R₄ are H, X=—N═N—, m=n=0, Q is not 4-carboxyphenyl; and
(21) when R₁ through R₄ are H, X=—N═N—, m=n=0, Q is not 2-hydroxy-1-naphthalenyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (4) are set forth in Table 4 below.

TABLE 4
Representative Compounds in Formula (4):

| Com- | | | | | | | | | |
|---|---|---|---|---|---|---|---|---|---|
| pound | | | | | | | | | |
| No. | R₁ | R₂ | R₃ | R₄ | R₅ | R₆ | —X— | m | n |
| 107. | H | H | H | H | | | —CO—O— | 0 | 0 |
| 108. | H | H | H | H | | | —CO—O— | 0 | 1 |
| 109. | H | H | H | H | | | —CO—O— | 0 | 2 |
| 110. | H | H | H | H | | | —CO—O— | 0 | 0 |
| 111. | H | H | H | H | | | —CO—O— | 0 | 1 |
| 112. | H | H | H | H | | | —CO—O— | 0 | 2 |
| 113. | H | H | H | H | | | —CO—NH— | 0 | 0 |
| 114. | H | H | H | H | | | —CO—NH— | 0 | 1 |
| 115. | H | H | H | H | | | —CO—NH— | 0 | 2 |
| 116. | H | H | H | H | | | —CO—NH— | 0 | 0 |
| 117. | H | H | H | H | | | —CO—NH— | 0 | 1 |
| 118. | H | H | H | H | | | —CO—NH— | 0 | 2 |
| 119. | H | H | H | H | | | —CO—NH— | 0 | 0 |
| 120. | H | H | H | H | | | —CO—NH— | 0 | 1 |
| 121. | H | H | H | H | | | —CO—NH— | 0 | 2 |
| 122. | H | H | H | H | | | —CO—NH— | 0 | 0 |
| 123. | H | H | H | H | | | —CO—NH— | 0 | 1 |
| 124. | H | H | H | H | | | —CO—NH— | 0 | 2 |
| 125. | H | H | H | H | | | —CO—NH— | 0 | 0 |
| 126. | H | H | H | H | | | —CO—NH— | 0 | 1 |
| 127. | H | H | H | H | | | —CO—NH— | 0 | 2 |
| 128. | H | H | H | H | | | —CO—NH— | 0 | 0 |
| 129. | H | H | H | H | | | —CO—NH— | 0 | 1 |
| 130. | H | H | H | H | | | —CO—NH— | 0 | 2 |
| 131. | H | H | H | H | | | —CO—NH— | 0 | 0 |
| 132. | H | H | H | H | | | —CO—NH— | 0 | 1 |
| 133. | H | H | H | H | | | —CO—NH— | 0 | 2 |
| 134. | H | H | H | H | | | —CO—NH— | 0 | 0 |
| 135. | H | H | H | H | | | —CO—NH— | 0 | 1 |
| 136. | H | H | H | H | | | —CO—NH— | 0 | 2 |
| 137. | H | H | H | H | | | 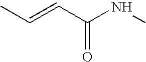 | 0 | 0 |
| 138. | H | H | H | H | | | 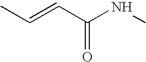 | 0 | 0 |
| 139. | H | H | H | H | | | 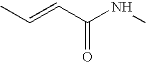 | 0 | 0 |
| 140. | H | H | H | H | | | 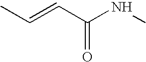 | 0 | 0 |
| 141. | H | H | H | H | | | 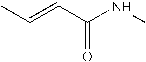 | 0 | 0 |
| 142. | H | H | H | H | | | 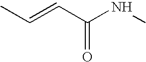 | 0 | 0 |
| 143. | H | H | H | H | | | 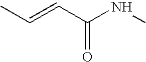 | 0 | 0 |
| 144. | H | H | H | H | | | 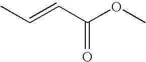 | 0 | 0 |
| 145. | H | H | H | H | | | 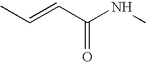 | 0 | 0 |
| 146. | H | H | H | H | | | 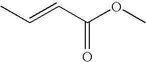 | 0 | 0 |
| 147. | H | F | H | H | | | —CO—O— | 0 | 0 |

| Compound | |
|---|---|
| No. | —Q |
| 107. | 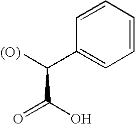 |
| 108. | 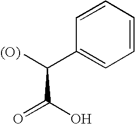 |
| 109. | 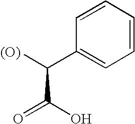 |
| 110. | 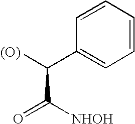 |
| 111. | 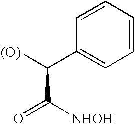 |
| 112. | 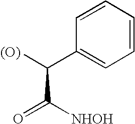 |
| 113. | 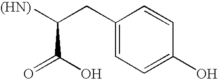 |
| 114. | 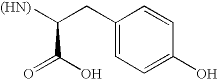 |
| 115. | 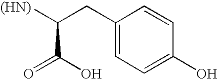 |
| 116. | 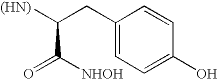 |
| 117. | 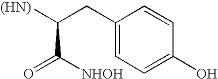 |
| 118. | 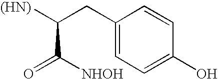 |
| 119. | 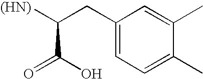 |
| 120. | 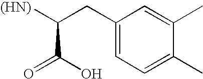 |
| 121. | 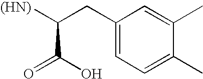 |
| 122. | 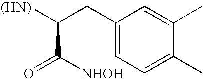 |
| 123. | 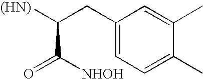 |
| 124. | 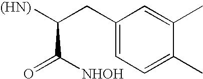 |
| 125. | 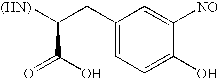 |
| 126. | 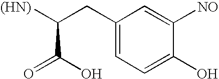 |
| 127. | 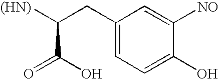 |
| 128. | 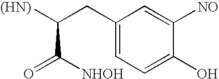 |
| 129. | 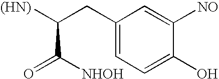 |
| 130. | 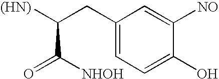 |
| 131. | 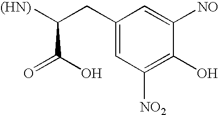 |
| 132. | 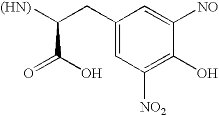 |
| 133. | 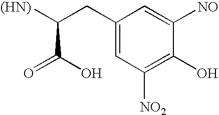 |
| 134. | 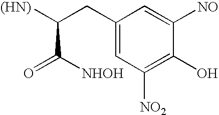 |
| 135. | 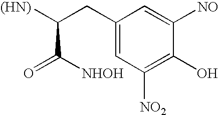 |
| 136. | 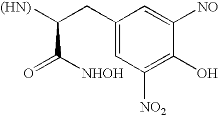 |
| 137. | 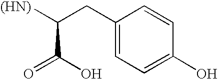 |
| 138. | 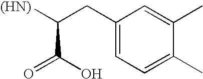 |
| 139. | 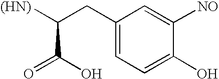 |
| 140. | 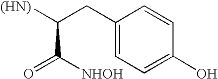 |
| 141. | 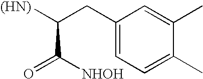 |
| 142. | 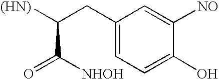 |
| 143. | 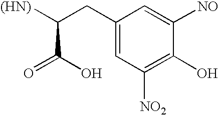 |
| 144. | 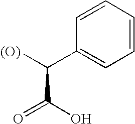 |
| 145. | 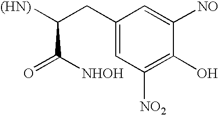 |
| 146. | 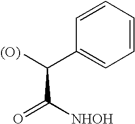 |
| 147. | 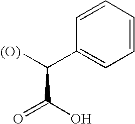 |

A most preferred compound in Formula (4) is: 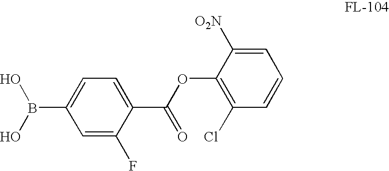
Additional compounds in Formula (4) are:

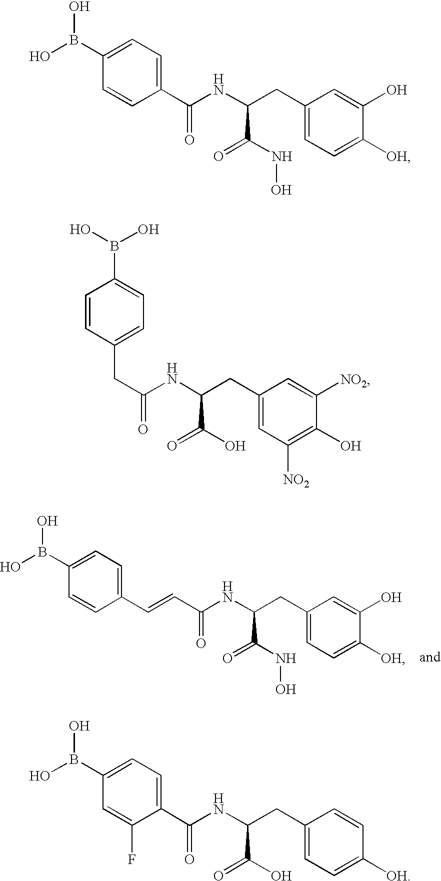

### 5. Fifth Preferred Embodiment

In a fifth preferred embodiment, the compounds are described by formula (5): 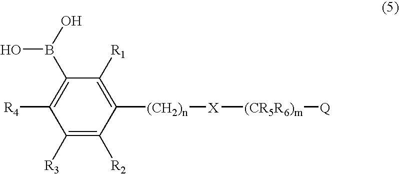
wherein R₁ through R₆, m, n, Q, and X can vary in order to optimize affinity, activity, absorption, distribution, metabolism, excretion, pharmacokinetic, toxicological and other properties required for their use as orally deliverable pharmaceuticals.

### a. Preferred Embodiment 5-a

In a more preferred fifth embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, formyl;
m and n is each independently 0, 1 or 2;
X is —O—, —NH—, —S—, —SO₂—, —CO—, —CH₂—, —CH═CH—CO—O—, —CH═CH—CO—NH—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁, represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 5-b

In another more preferred fifth embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0 or 1;
X is —CO—O—, —CH═CH—CO—O—, —O—CO—, —CO—NH—, —CH═CH—CO—NH—, —NH—CO—, —CH═N—, —NH—CH₂—, —CH₂—NH—, —SO₂—O—, —O—SO₂—, —SO₂—NH— or —NH—SO₂—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

### c. Preferred Embodiment 5-c

In another more preferred fifth embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, formyl;
m and n is each independently 0, 1 or 2;
X is —O—, —NH—, —S—, —SO₂—, —CO—, —CH₂—, —CH═CH—CO—O—, —CH═CH—CO—NH—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
with the proviso that,
(1) when m=0 and Q is 3-boronophenyl, X is not —CO—NH or —SO₂—NH—;
(2) when R₂ and R₃ are H, m=n=0, X is not —CH═CH—;
(3) when m=0, n=0 and Q is a benzene, a benzocyclic alkene or a benzoheterocyclic alkene, which may be substituted with one or more substituents R₁₃, X is not —CO—O—;
(4) when m=0, n=0 and Q is a benzene, a benzocyclic alkene or a benzoheterocyclic alkene, which may be substituted with one or more substituents R₁₃, X is not —NH—CO—;
(5) when m=0, n=0 and Q is a benzene, a benzocyclic alkene or a benzoheterocyclic alkene, which may be substituted with one or more substituents R₁₃, X is not —O—CO—;
(6) when R₅ and R₆=H, m=1, n=0 and Q is a benzene, a benzocyclic alkene or a benzoheterocyclic alkene, which may be substituted with one or more substituents R₁₃, X is not —CO—O—;
(7) when R₁ through R₄ are H, X=—CO—NH—, m=n=0, Q is not phenyl;
(8) when R₁ and R₂ are H, R₃ is F, R₄ is methyl, X=—CO—NH—, m=n=0, Q is not cyclopropyl;
(9) when R₁ through R₆ are H, X=—CO—NH—, m=3, n=0, Q is not phenyl;
(10) when R₁ through R₆ are H, X=—CH₂—CH—, m=1, n=0, Q is not phenyl;
(11) when R₁ through R₄ are H, X=—NH—CH₂—, m=0 and n=1, Q is not phenyl;
(12) when R₁ through R₄ are H, X=—NH—CH₂—, m=0 and n=0, Q is not pentafluorophenyl;
(13) when R₁ through R₄ are H, X=—CH═CH—, m=0 and n=0, Q is not phenyl; and
(14) when R₁ through R₄ are H, X=—CH═CH—, m=0 and n=0, Q is not 2-boronophenyl.

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### d. Preferred Embodiment 5-d

In another more preferred fifth embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0 or 1;
X is —CO—O—, —CH═CH—CO—O—, —O—CO—, —CO—NH—, —CH═CH—CO—NH—, —NH—CO—, —CH═N—, —NH—CH₂—, —CH₂—NH—, —SO₂—O—, —O—SO₂—, —SO₂—NH— or —NH—SO₂—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
with the proviso that,
(1) when m=0 and Q is 3-boronophenyl, X is not —CO—NH or —SO₂—NH—;
(2) when R and R₃ are H, m=n=0, X is not —CH═CH—,
(3) when m=0, n=0 and Q is a benzene, a benzocyclic alkene or a benzoheterocyclic alkene, which may be substituted with one or more substituents R₁₃, X is not —CO—O—;
(4) when m=0, n=0 and Q is a benzene, a benzocyclic alkene or a benzoheterocyclic alkene, which may be substituted with one or more substituents R₁₃, X is not —NH—CO—;
(5) when m=0, n=0 and Q is a benzene, a benzocyclic alkene or a benzoheterocyclic alkene, which may be substituted with one or more substituents R₁₃, X is not —O—CO—;
(6) when R₅ and R₆=H, m=1, n=0 and Q is a benzene, a benzocyclic alkene or a benzoheterocyclic alkene, which may be substituted with one or more substituents R₁₃, X is not —CO—O—;
(7) when R₁ through R₄ are H, X=—CO—NH—, m=n=0, Q is not phenyl;
(8) when R₁ and R₂ are H, R₃ is F, R₄ is methyl, X=—CO—NH—, m=n=0, Q is not cyclopropyl;
(9) when R₁ through R₆ are H, X=—CO—NH—, m=3, n=0, Q is not phenyl;
(10) when R₁ through R₆ are H, X=—CH₂—CH—, m=1, n=0, Q is not phenyl;
(11) when R₁ through R₄ are H, X=—NH—CH₂—, m=0 and n=1, Q is not phenyl;
(12) when R₁ through R₄ are H, X=—NH—CH₂—, m=0 and n=0, Q is not pentafluorophenyl;
(13) when R₁ through R₄ are H, X=—CH═CH—, m=0 and n=0, Q is not phenyl; and
(14) when R₁ through R₄ are H, X=—CH═CH—, m=0 and n=0, Q is not 2-boronophenyl.

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (5) are set forth in Table 5 below.

TABLE 5
Representative Compounds in Formula (5):

| Compound | | | | | | | | | | |
|---|---|---|---|---|---|---|---|---|---|---|
| No. | R₁ | R₂ | R₃ | R₄ | R₅ | R₆ | -X- | m | n | -Q |
| 148. | H | H | —COOH | H | | | —NH—CO— | 0 | 0 | 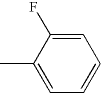 |
| 149. | H | H | —NO₂ | H | H | H | —CO—O— | 1 | 0 | 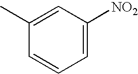 |
| 150. | H | H | —NO₂ | H | | | —CO—O— | 0 | 0 | 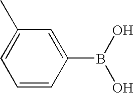 |
| 151. | H | H | —NO₂ | H | H | H | —CO—O— | 1 | 0 | 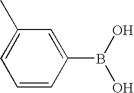 |
| 152. | H | H | H | H | | | —CO—O— | 0 | 0 | 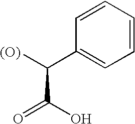 |
| 153. | H | H | H | H | | | —CO—O— | 0 | 1 | 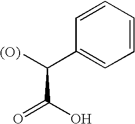 |
| 154. | H | H | H | H | | | —CO—O— | 0 | 2 | 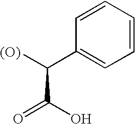 |
| 155. | H | H | H | H | | | —CO—O— | 0 | 0 | 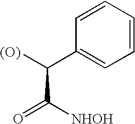 |
| 156. | H | H | H | H | | | —CO—O— | 0 | 1 | 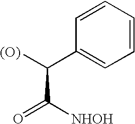 |
| 157. | H | H | H | H | | | —CO—O— | 0 | 2 | 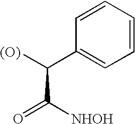 |
| 158. | H | H | H | H | | | —CO—NH— | 0 | 0 | 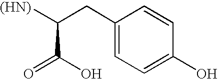 |
| 159. | H | H | H | H | | | —CO—NH— | 0 | 1 | 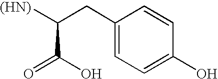 |
| 160. | H | H | H | H | | | —CO—NH— | 0 | 2 | 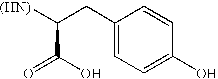 |
| 161. | H | H | H | H | | | —CO—NH— | 0 | 0 | 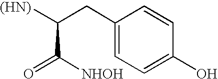 |
| 162. | H | H | H | H | | | —CO—NH— | 0 | 1 | 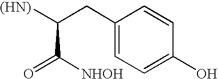 |
| 163. | H | H | H | H | | | —CO—NH— | 0 | 2 | 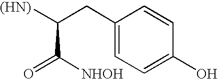 |
| 164. | H | H | H | H | | | —CO—NH— | 0 | 0 | 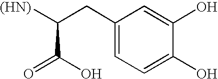 |
| 165. | H | H | H | H | | | —CO—NH— | 0 | 1 | 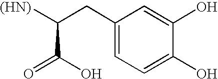 |
| 166. | H | H | H | H | | | —CO—NH— | 0 | 2 | 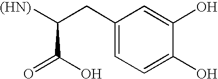 |
| 167. | H | H | H | H | | | —CO—NH— | 0 | 0 | 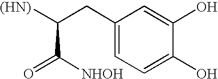 |
| 168. | H | H | H | H | | | —CO—NH— | 0 | 1 | 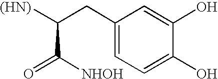 |
| 169. | H | H | H | H | | | —CO—NH— | 0 | 2 | 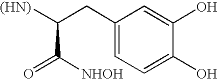 |
| 170. | H | H | H | H | | | —CO—NH— | 0 | 0 | 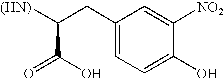 |
| 171. | H | H | H | H | | | —CO—NH— | 0 | 1 | 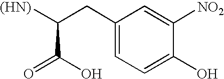 |
| 172. | H | H | H | H | | | —CO—NH— | 0 | 2 | 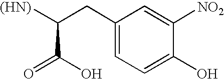 |
| 173. | H | H | H | H | | | —CO—NH— | 0 | 0 | 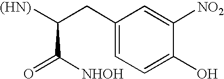 |
| 174. | H | H | H | H | | | —CO—NH— | 0 | 1 | 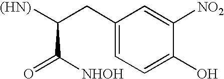 |
| 175. | H | H | H | H | | | —CO—NH— | 0 | 2 | 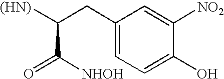 |
| 176. | H | H | H | H | | | —CO—NH— | 0 | 0 | 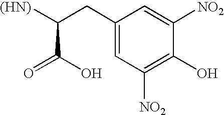 |
| 177. | H | H | H | H | | | —CO—NH— | 0 | 1 | 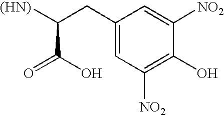 |
| 178. | H | H | H | H | | | —CO—NH— | 0 | 2 | 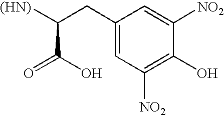 |
| 179. | H | H | H | H | | | —CO—NH— | 0 | 0 | 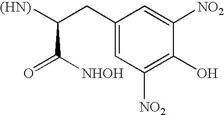 |
| 180. | H | H | H | H | | | —CO—NH— | 0 | 1 | 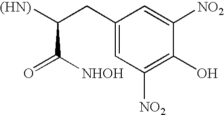 |
| 181. | H | H | H | H | | | —CO—NH— | 0 | 2 | 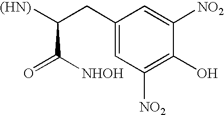 |
| 182. | H | H | H | H | | | 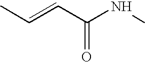 | 0 | 0 | 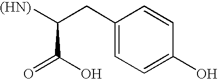 |
| 183. | H | H | H | H | | | 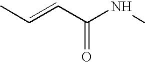 | 0 | 0 | 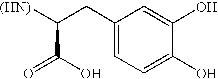 |
| 184. | H | H | H | H | | | 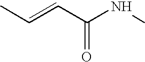 | 0 | 0 | 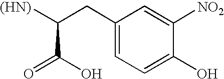 |
| 185. | H | H | H | H | | | 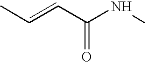 | 0 | 0 | 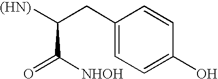 |
| 186. | H | H | H | H | | | 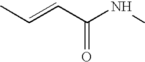 | 0 | 0 | 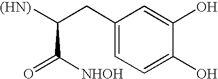 |
| 187. | H | H | H | H | | | 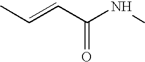 | 0 | 0 | 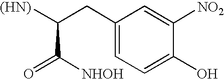 |
| 188. | H | H | H | H | | | 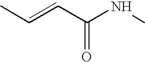 | 0 | 0 | 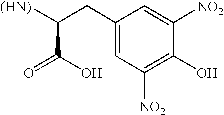 |
| 189. | H | H | H | H | | | 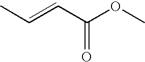 | 0 | 0 | 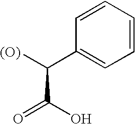 |
| 190. | H | H | H | H | | | 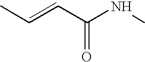 | 0 | 0 | 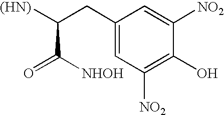 |
| 191. | H | H | H | H | | | 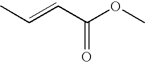 | 0 | 0 | 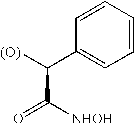 |
| 192. | H | H | —NO₂ | H | | | —CO—O— | 0 | 0 | 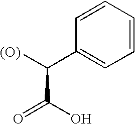 |
| 193. | H | H | —NO₂ | H | | | —CO—O— | 0 | 1 | 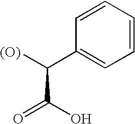 |
| 194. | H | H | —NO₂ | H | | | —CO—O— | 0 | 2 | 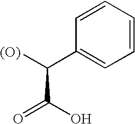 |
| 195. | H | H | —NO₂ | H | | | —CO—O— | 0 | 0 | 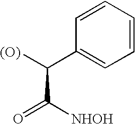 |
| 196. | H | H | —NO₂ | H | | | —CO—O— | 0 | 1 | 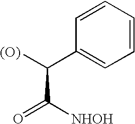 |
| 197. | H | H | —NO₂ | H | | | —CO—O— | 0 | 2 | 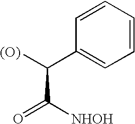 |
| 198. | H | H | —NO₂ | H | | | —CO—NH— | 0 | 0 | 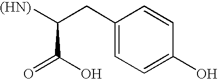 |
| 199. | H | H | —NO₂ | H | | | —CO—NH— | 0 | 1 | 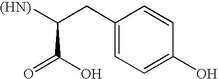 |
| 200. | H | H | —NO₂ | H | | | —CO—NH— | 0 | 2 | 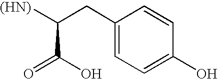 |
| 201. | H | H | —NO₂ | H | | | —CO—NH— | 0 | 0 | 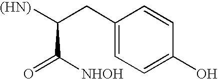 |
| 202. | H | H | —NO₂ | H | | | —CO—NH— | 0 | 1 | 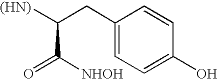 |
| 203. | H | H | —NO₂ | H | | | —CO—NH— | 0 | 2 | 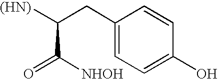 |
| 204. | H | H | —NO₂ | H | | | —CO—NH— | 0 | 0 | 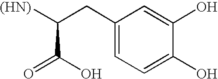 |
| 205. | H | H | —NO₂ | H | | | —CO—NH— | 0 | 1 | 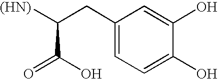 |
| 206. | H | H | —NO₂ | H | | | —CO—NH— | 0 | 2 | 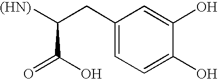 |
| 207. | H | H | —NO₂ | H | | | —CO—NH— | 0 | 0 | 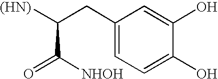 |
| 208. | H | H | —NO₂ | H | | | —CO—NH— | 0 | 1 | 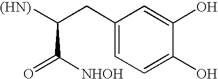 |
| 209. | H | H | —NO₂ | H | | | —CO—NH— | 0 | 2 | 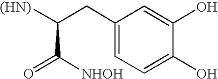 |
| 210. | H | H | —NO₂ | H | | | —CO—NH— | 0 | 0 | 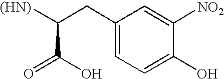 |
| 211. | H | H | —NO₂ | H | | | —CO—NH— | 0 | 1 | 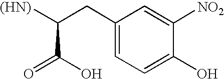 |
| 212. | H | H | —NO₂ | H | | | —CO—NH— | 0 | 2 | 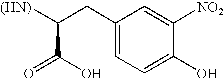 |
| 213. | H | H | —NO₂ | H | | | —CO—NH— | 0 | 0 | 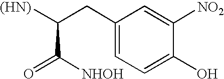 |
| 214. | H | H | —NO₂ | H | | | —CO—NH— | 0 | 1 | 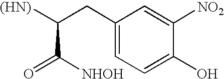 |
| 215. | H | H | —NO₂ | H | | | —CO—NH— | 0 | 2 | 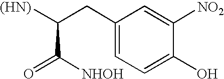 |
| 216. | H | H | —NO₂ | H | | | —CO—NH— | 0 | 0 | 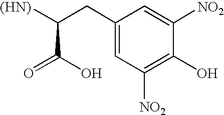 |
| 217. | H | H | —NO₂ | H | | | —CO—NH— | 0 | 1 | 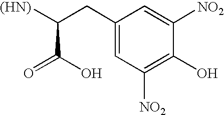 |
| 218. | H | H | —NO₂ | H | | | —CO—NH— | 0 | 2 | 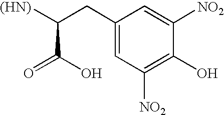 |
| 219. | H | H | —NO₂ | H | | | —CO—NH— | 0 | 0 | 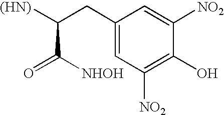 |
| 220. | H | H | —NO₂ | H | | | —CO—NH— | 0 | 1 | 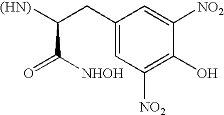 |
| 221. | H | H | —NO₂ | H | | | —CO—NH— | 0 | 2 | 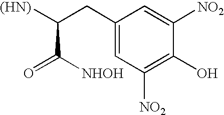 |
| 222. | H | H | —NO₂ | H | | | 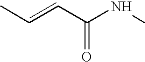 | 0 | 0 | 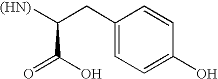 |
| 223. | H | H | —NO₂ | H | | | 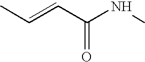 | 0 | 0 | 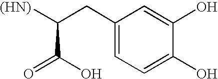 |
| 224. | H | H | —NO₂ | H | | | 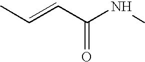 | 0 | 0 | 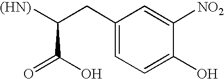 |
| 225. | H | H | —NO₂ | H | | | 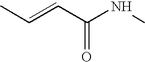 | 0 | 0 | 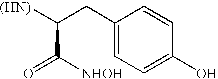 |
| 226. | H | H | —NO₂ | H | | | 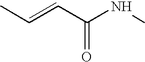 | 0 | 0 | 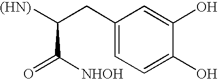 |
| 227. | H | H | —NO₂ | H | | | 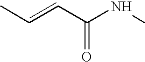 | 0 | 0 | 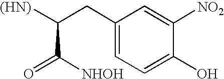 |
| 228. | H | H | —NO₂ | H | | | 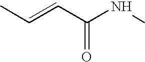 | 0 | 0 | 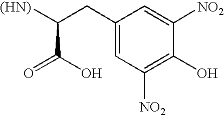 |
| 229. | H | H | —NO₂ | H | | | 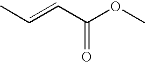 | 0 | 0 | 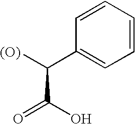 |
| 230. | H | H | —NO₂ | H | | | 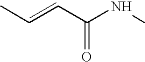 | 0 | 0 | 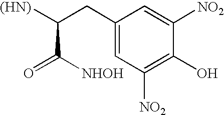 |
| 231. | H | H | —NO₂ | H | | | 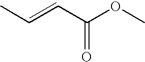 | 0 | 0 | 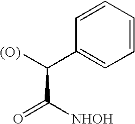 |

The most preferred compounds in Formula (5) are:

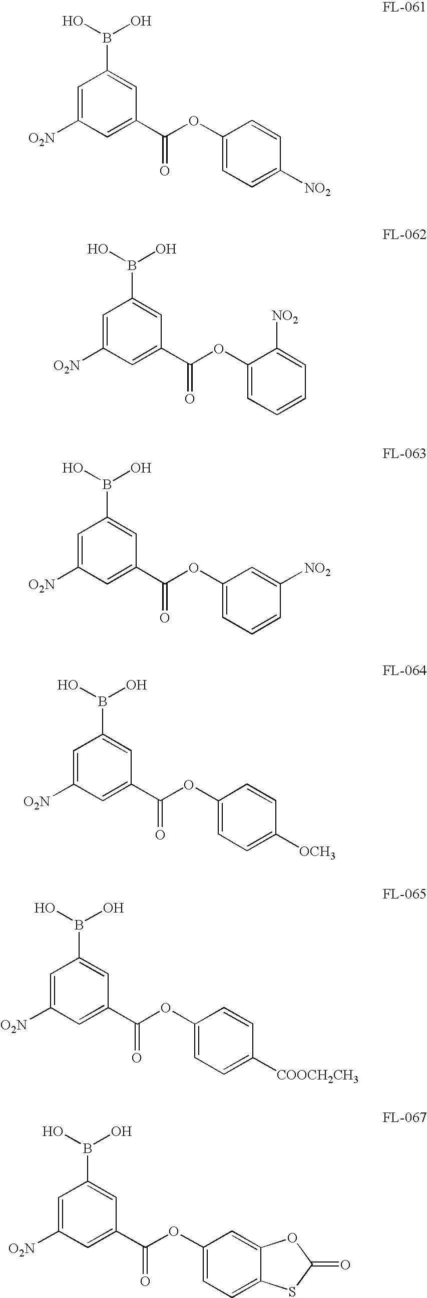

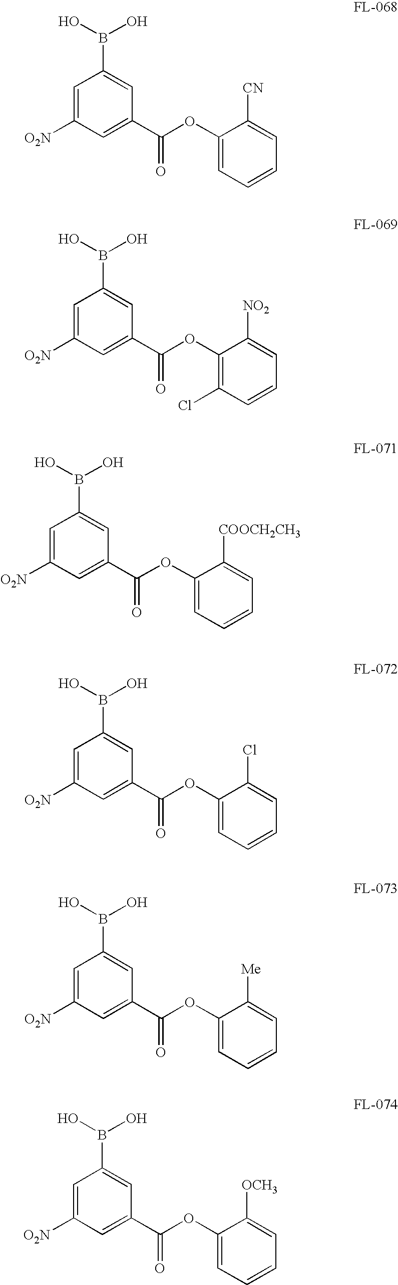

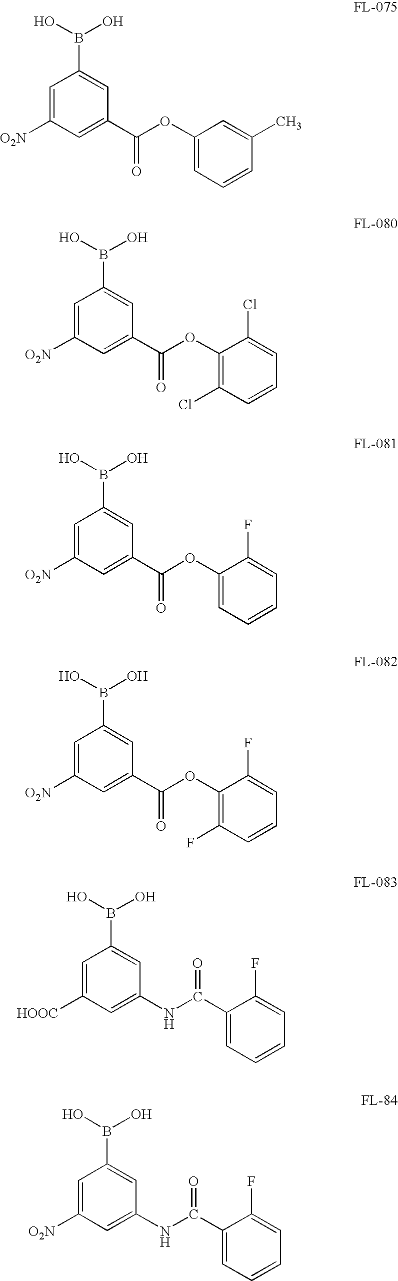

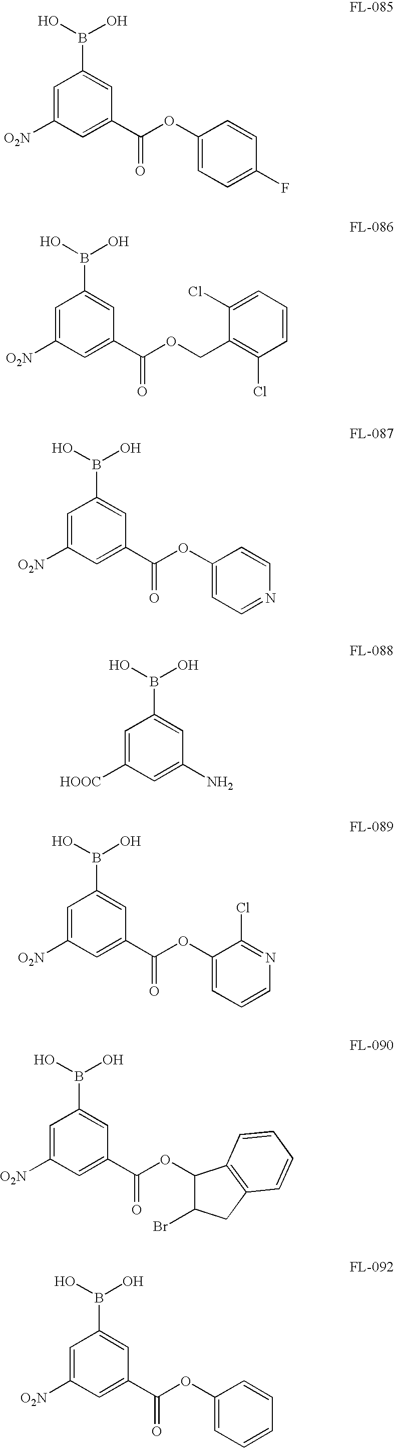

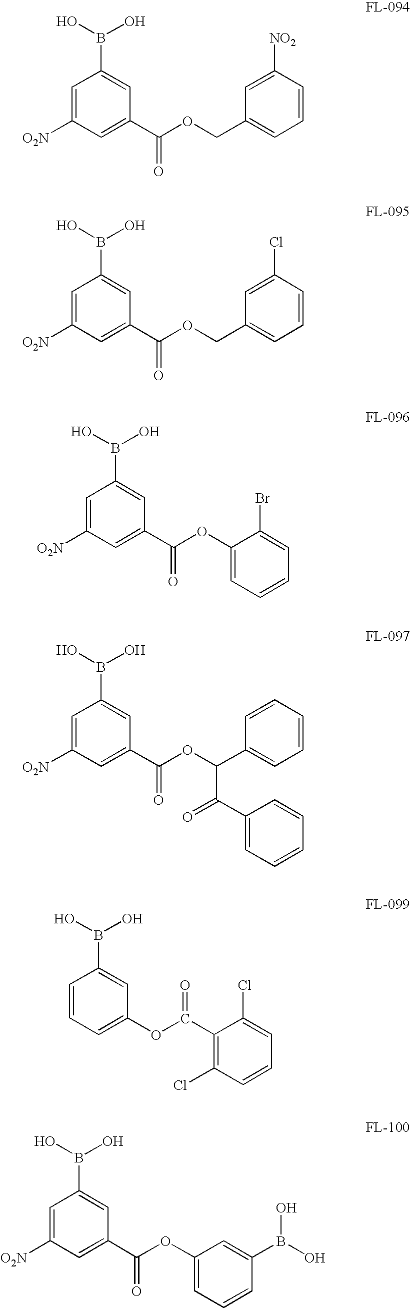

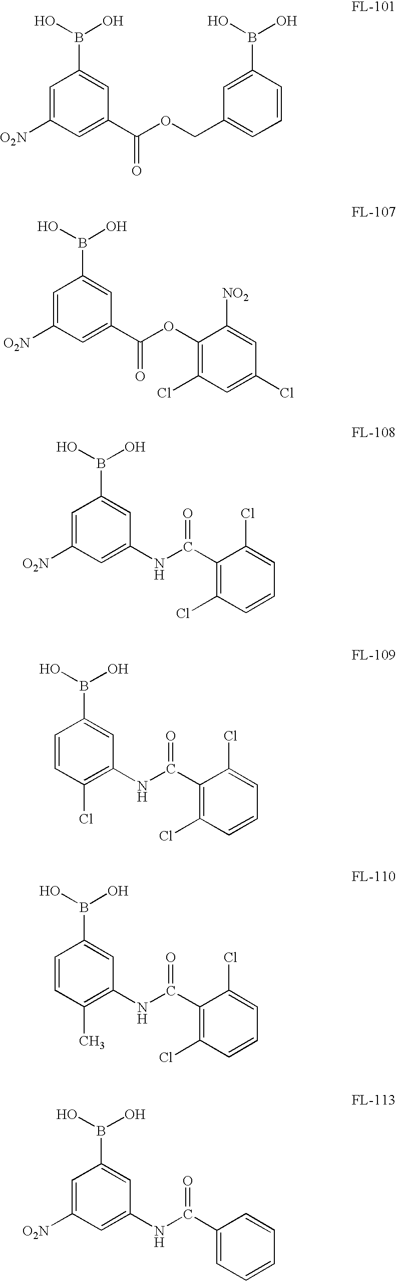

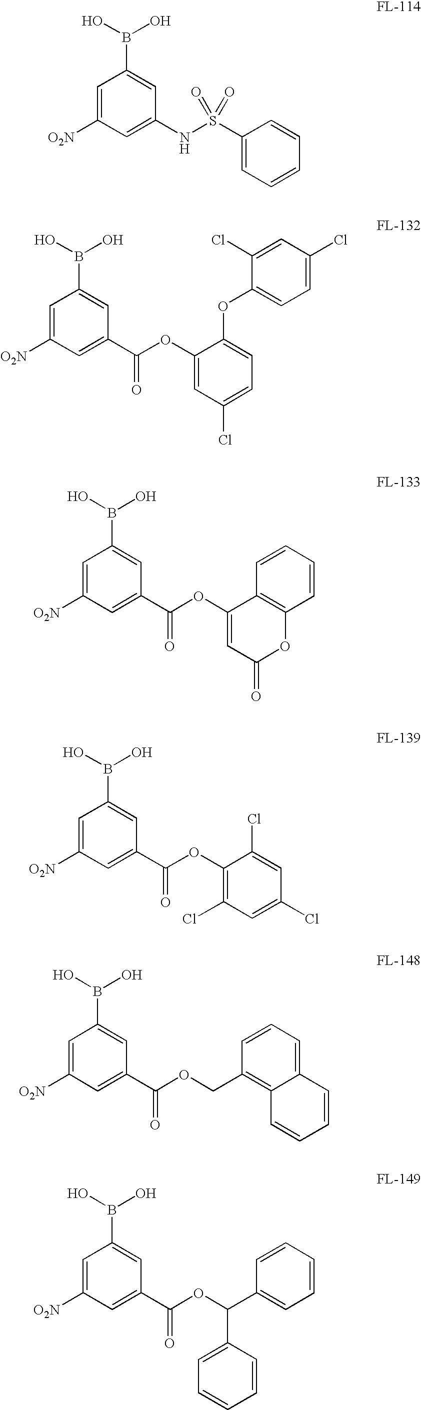

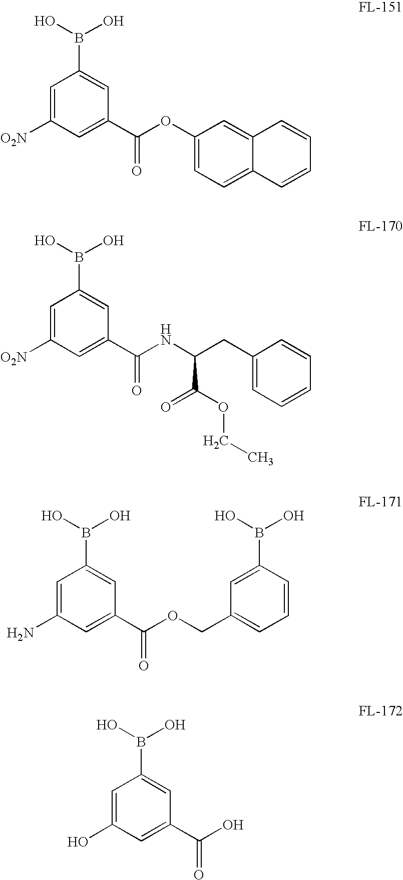

Additional compounds in Formula (5) are:

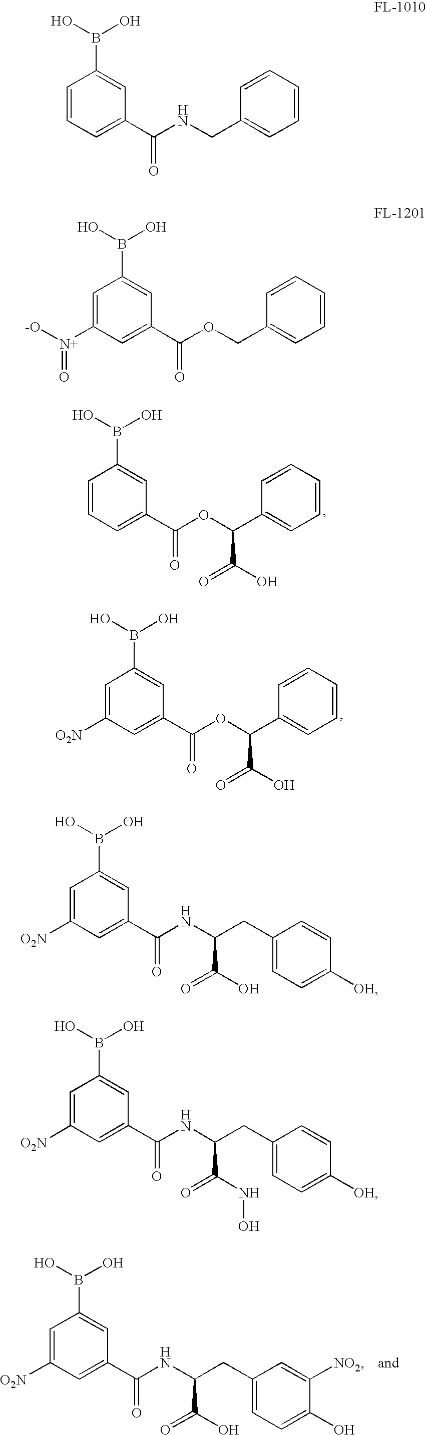

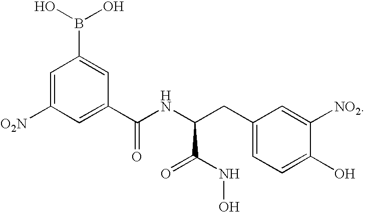

### 6. Sixth Preferred Embodiment

In a sixth preferred embodiment, the compounds are described by formula (6): 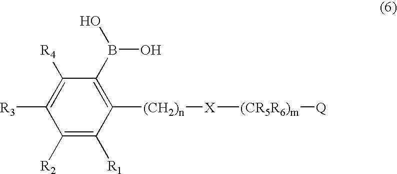
wherein R₁ through R₆, m, n, Q and X can vary in order to optimize affinity, activity, absorption, distribution, metabolism, excretion, pharmacokinetic, toxicological and other properties required for their use as orally deliverable pharmaceuticals.

### a. Preferred Embodiment 6-a

In a more preferred sixth embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0, 1 or 2;
X is —O—, —NH—, —S—, —SO₂—, —CO—, —CH₂—, —CH═CH—CO—O—, —CH═CH—CO—NH—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 6-b

In another more preferred sixth embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, —SO₂NH₂;
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0 or 1;
X is —CO—O—, —CH═CH—CO—O—, —O—CO—, —CO—NH—, —CH═CH—CO—NH—, —NH—CO—, —CH═N—, —CH═CH—, —NH—CH₂—, —CH₂—NH—, —CH₂—O—, —SO₂—O—, —O—SO₂—, —SO₂—NH—, or —NH—SO₂—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

### c. Preferred Embodiment 6-c

In another more preferred sixth embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0, 1 or 2;
X is —O—, —NH—, —S—, —SO₂—, —CO—, —CH₂—, —CH═CH—CO—O—, —CH═CH—CO—NH—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₁ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
with the proviso that,
(1) when R₁ through R₄ are H, X is not —CH₂—NH—;
(2) when m=0 and Q is 3-boronophenyl, X is not —CO—NH or —SO₂—NH—;
(3) when R₁ through R₄ are H, X=—NH—CO—, m=0 and n=1, Q is not phenyl;
(4) when R₁ through R₄ are H, X=—NH—CO—, m=0 and n=0, Q is not phenyl;
(5) when R₁ through R₄ are H, X=—NH—CH₂—, m=0 and n=1, Q is not phenyl;
(6) when R₁ through R₆ are H, X=—NH—CH₂—, m=2 and n=1, Q is not phenyl;
(7) when R₁ through R₄ are H, X=—NH—CH₂—, m=0 and n=1, Q is not 10-(hydroxymethyl)-9-anthracenyl;
(8) when R₁ through R₄ are H, X=—NH—CH₂—, m=n=0, Q is not phenyl;
(9) when R₁ through R₈ are H, X=—CH₂—NH—, m=n=1, U is 1,3-benzene, Y=Z=—NH—CH₂—, Q is not 2-boronophenyl; and
(10) when R₁ through R₆ are H, X=—CO—O—, m=1 and n=0, Q is not 4-methoxyphenyl.

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### d. Preferred Embodiment 6-d

In another more preferred sixth embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, —SO₂NH₂;
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0 or 1;
X is —CO—O—, —CH═CH—CO—O—, —O—CO—, —CO—NH—, —CH═CH—CO—NH—, —NH—CO—, —CH═N—, —CH═CH—, —NH—CH₂—, —CH₂—NH—, —CH₂—O—, —SO₂—O—, —O—SO₂—, —SO₂—NH—, or —NH—SO₂—;
Q represents —CH₂CHR₁₁COR₁₂ or —CHR₁₁COR₁₂ [wherein R₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
with the proviso that,
(1) when R₁ through R₄ are H, X is not —CH₂—NH—;
(2) when m=0 and Q is 3-boronophenyl, X is not —CO—NH or —SO₂—NH—;
(3) when R₁ through R₄ are H, X=—NH—CO—, m=0 and n=1, Q is not phenyl;
(4) when R₁ through R₄ are H, X=—NH—CO—, m=0 and n=0, Q is not phenyl;
(5) when R₁ through R₄ are H, X=—NH—CH₂—, m=0 and n=1, Q is not phenyl;
(6) when R₁ through R₆ are H, X=—NH—CH₂—, m=2 and n=1, Q is not phenyl;
(7) when R₁ through R₄ are H, X=—NH—CH₂—, m=0 and n=1, Q is not 10-(hydroxymethyl)-9-anthracenyl;
(8) when R₁ through R₄ are H, X=—NH—CH₂—, m=n=0, Q is not phenyl;
(9) when R₁ through R₈ are H, X=—CH₂—NH—, m=n=1, U is 1,3-benzene, Y=Z=—NH—CH₂—, Q is not 2-boronophenyl; and
(10) when R₁ through R₆ are H, X=—CO—O—, m=1 and n=0, Q is not 4-methoxyphenyl.

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (6) are set forth in Table 6 below.

TABLE 6
Representative Compounds in Formula (6):

| Compound | | | | | | | | | | |
|---|---|---|---|---|---|---|---|---|---|---|
| No. | R₁ | R₂ | R₃ | R₄ | R₅ | R₆ | -X- | m | n | -Q |
| 232. | H | H | H | H | | | —CO—O— | 0 | 0 | 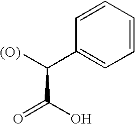 |
| 233. | H | H | H | H | | | —CO—O— | 0 | 1 | 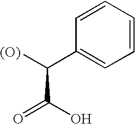 |
| 234. | H | H | H | H | | | —CO—O— | 0 | 2 | 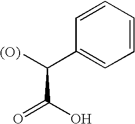 |
| 235. | H | H | H | H | | | —CO—O— | 0 | 0 | 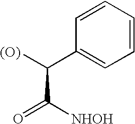 |
| 236. | H | H | H | H | | | —CO—O— | 0 | 1 | 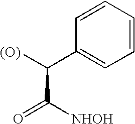 |
| 237. | H | H | H | H | | | —CO—O— | 0 | 2 | 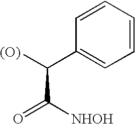 |
| 238. | H | H | H | H | | | —CO—NH— | 0 | 0 | 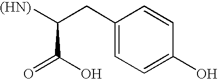 |
| 239. | H | H | H | H | | | —CO—NH— | 0 | 1 | 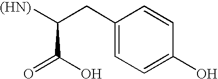 |
| 240. | H | H | H | H | | | —CO—NH— | 0 | 2 | 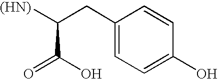 |
| 241. | H | H | H | H | | | —CO—NH— | 0 | 0 | 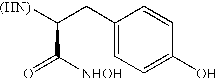 |
| 242. | H | H | H | H | | | —CO—NH— | 0 | 1 | 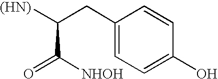 |
| 243. | H | H | H | H | | | —CO—NH— | 0 | 2 | 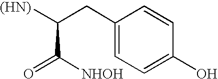 |
| 244. | H | H | H | H | | | —CO—NH— | 0 | 0 | 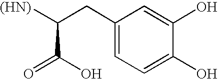 |
| 245. | H | H | H | H | | | —CO—NH— | 0 | 1 | 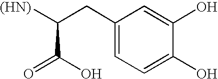 |
| 246. | H | H | H | H | | | —CO—NH— | 0 | 2 | 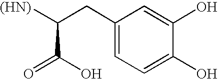 |
| 247. | H | H | H | H | | | —CO—NH— | 0 | 0 | 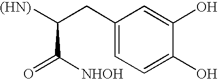 |
| 248. | H | H | H | H | | | —CO—NH— | 0 | 1 | 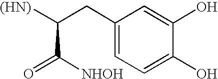 |
| 249. | H | H | H | H | | | —CO—NH— | 0 | 2 | 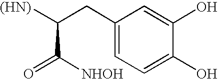 |
| 250. | H | H | H | H | | | —CO—NH— | 0 | 0 | 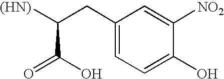 |
| 251. | H | H | H | H | | | —CO—NH— | 0 | 1 | 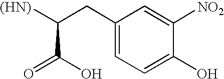 |
| 252. | H | H | H | H | | | —CO—NH— | 0 | 2 | 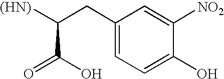 |
| 253. | H | H | H | H | | | —CO—NH— | 0 | 0 | 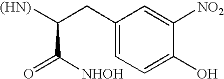 |
| 254. | H | H | H | H | | | —CO—NH— | 0 | 1 | 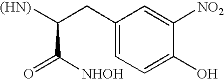 |
| 255. | H | H | H | H | | | —CO—NH— | 0 | 2 | 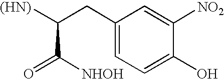 |
| 256. | H | H | H | H | | | —CO—NH— | 0 | 0 | 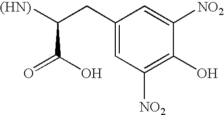 |
| 257. | H | H | H | H | | | —CO—NH— | 0 | 1 | 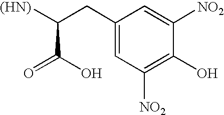 |
| 258. | H | H | H | H | | | —CO—NH— | 0 | 2 | 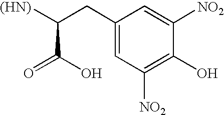 |
| 259. | H | H | H | H | | | —CO—NH— | 0 | 0 | 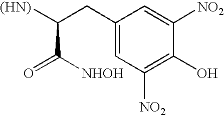 |
| 260. | H | H | H | H | | | —CO—NH— | 0 | 1 | 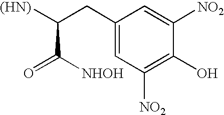 |
| 261. | H | H | H | H | | | —CO—NH— | 0 | 2 | 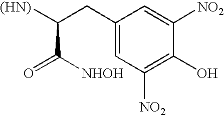 |
| 262. | H | H | H | H | | | 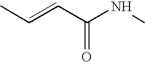 | 0 | 0 | 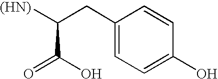 |
| 263. | H | H | H | H | | | 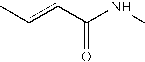 | 0 | 0 | 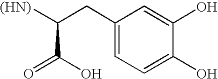 |
| 264. | H | H | H | H | | | 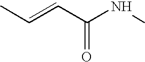 | 0 | 0 | 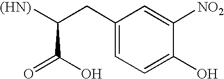 |
| 265. | H | H | H | H | | | 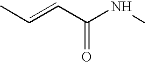 | 0 | 0 | 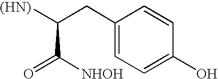 |
| 266. | H | H | H | H | | | 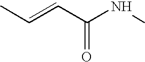 | 0 | 0 | 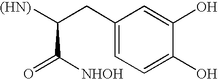 |
| 267. | H | H | H | H | | | 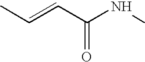 | 0 | 0 | 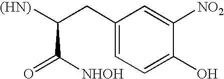 |
| 268. | H | H | H | H | | | 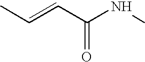 | 0 | 0 | 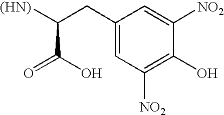 |
| 269. | H | H | H | H | | | 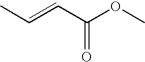 | 0 | 0 | 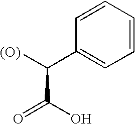 |
| 270. | H | H | H | H | | | 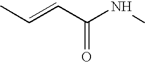 | 0 | 0 | 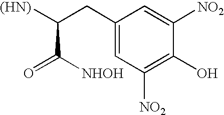 |
| 271. | H | H | H | H | | | 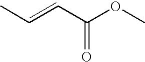 | 0 | 0 | 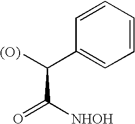 |

The most preferred compounds in Formula (6) are:

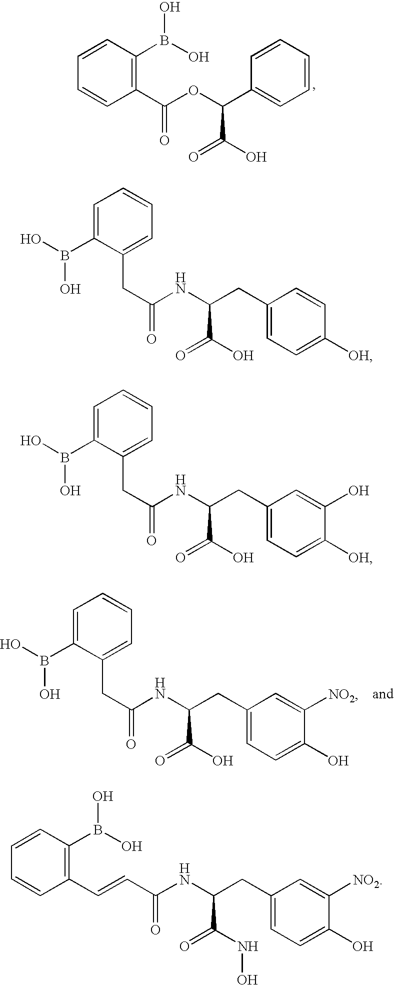

### 7. Seventh Preferred Embodiment

In a seventh preferred embodiment, the compounds are described by formula (7): 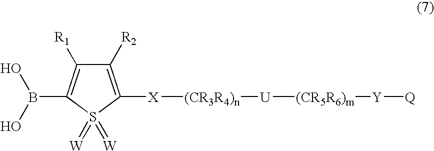
wherein R₁ through R₆, m, n, Q, U, W, X, and Y can vary in order to optimize affinity, activity, absorption, distribution, metabolism, excretion, pharmacokinetic, toxicological and other properties required for their use as orally deliverable pharmaceuticals.

### a. Preferred Embodiment 7-a

In a more preferred seventh embodiment,
R₁ and R₂ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₃ through R₆ each independently represents hydrogen, C₁₋₆ alkyl, C₃₋₇ cycloalkyl, benzyl or the carbon and attached two Rᵢs, they together form C₃₋₇ cycloalkyl;
m and n is each independently 0 or 1;
X and Y is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5 or 6, and j=0, 1 or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, —CH₂COOH, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₄₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 7-b

In another more preferred seventh embodiment,
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
R₃ through R₆ each independently represents hydrogen, C₁₋₆ alkyl, C₃₋₇ cycloalkyl, benzyl, or the carbon and attached two Rᵢs, they together form C₃₋₇ cycloalkyl;
m or n is each independently 0 or 1;
X is —CO—O—, —CH₂—O—CO—, —CO—NH—, —CH₂—O—NH—, —CH₂—CH₂, —CH═CH—, —CH₂—O—, —CH═N—, or —CH₂—NH—;
Y is —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N=═N—;
W is oxygen or lone-pair electrons;
U represents —(CH₂)ᵢ or —(CH═CH)ⱼ (wherein i=0, 1, 2 or 3 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (7) are set forth in Table 7 below.

TABLE 7
Representative Compounds in Formula 7:

| Compound | | | | | | | | | | | | | |
|---|---|---|---|---|---|---|---|---|---|---|---|---|---|
| No. | R₁ | R₂ | R₃ | R₄ | R₅ | R₆ | W | -X- | -Y- | m | n | -U- | -Q |
| 272. | H | H | H | H | H | H | | —CH₂O— | —OCH₂— | 1 | 1 | 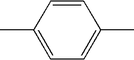 | 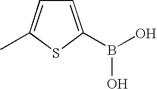 |
| 273. | H | H | | | | | | —CO—O— | —O—CO— | 0 | 0 | 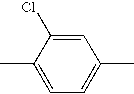 | 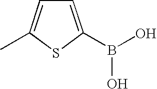 |
| 274. | H | H | | | | | | —CO—O— | —CO—O— | 0 | 0 | 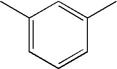 | 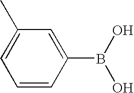 |
| 275. | H | H | | | | | | —CO—NH— | —CO—NH— | 0 | 0 | —CH₂— | 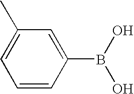 |
| 276. | H | H | | | | | | —CO—NH— | —CO—NH— | 0 | 0 | —CH₂— | 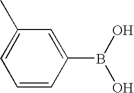 |
| 277. | H | H | | | | | | —CO—NH— | —CO—NH— | 0 | 0 | 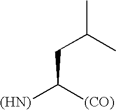 | 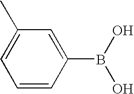 |
| 278. | H | H | | | | | | —CO—NH— | —CO—NH— | 0 | 0 | 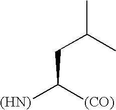 | 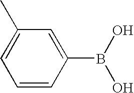 |
| 279. | H | H | H | H | H | H | O | —CH₂O— | —OCH₂— | 1 | 1 | 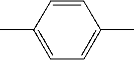 | 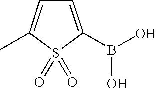 |
| 280. | H | H | | | | | O | —CO—O— | —O—CO— | 0 | 0 | 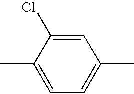 | 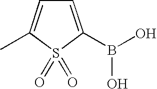 |
| 281. | H | H | | | | | O | —CO—O— | —CO—O— | 0 | 0 | 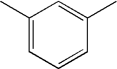 | 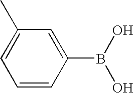 |
| 282. | H | H | | | | | O | —CO—NH— | —CO—NH— | 0 | 0 | —CH₂— | 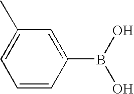 |
| 283. | H | H | | | | | O | —CO—NH— | —CO—NH— | 0 | 0 | —CH₂— | 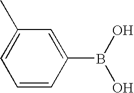 |
| 284. | H | H | | | | | O | —CO—NH— | —CO—NH— | 0 | 0 | 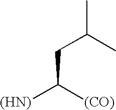 | 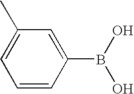 |
| 285. | H | H | | | | | O | —CO—NH— | —CO—NH— | 0 | 0 | 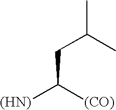 | 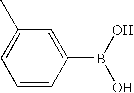 |

The most preferred compounds in Formula (7) are:

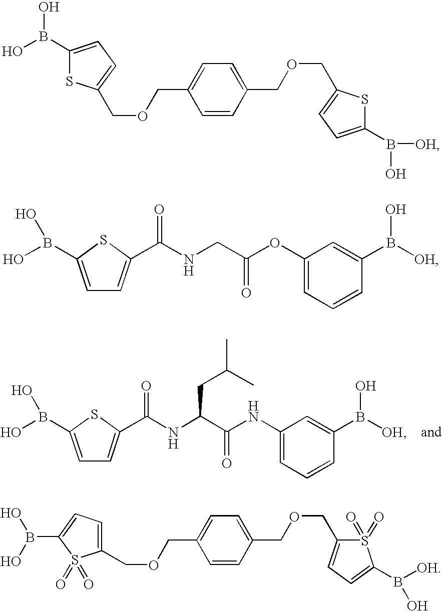

### 58. Eighth Preferred Embodiment

In an eighth preferred embodiment, the compounds are described by formula (8): 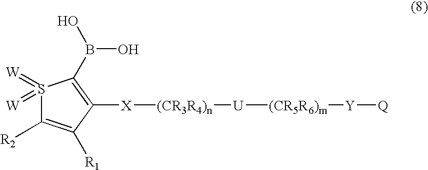
wherein R₁ through R₆, m, n, Q, U, V, W, X, and Y can vary in order to optimize affinity, activity, absorption, distribution, metabolism, excretion, pharmacokinetic, toxicological and other properties required for their use as orally deliverable pharmaceuticals.

### a. Preferred Embodiment 8-a

In a more preferred eighth embodiment,
R₁ and R₂ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₃ through R₆ each independently represents hydrogen, C₁₋₆ alkyl, C₃₋₇ cycloalkyl, benzyl or the carbon and attached two Rᵢs, they together form C₃₋₇ cycloalkyl;
m and n is each independently 0 or 1;
X and Y is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5 or 6, and j=0, 1 or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, —CH₂COOH, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 8-b

In another more preferred eighth embodiment,
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
R₃ through R₆ each independently represents hydrogen, C₁₋₆ alkyl, C₃₋₇ cycloalkyl, benzyl, or the carbon and attached two Rᵢs, they together form C₃₋₇ cycloalkyl;
m or n is each independently 0 or 1;
X is —CO—O—, —CH₂—O—CO—, —CO—NH—, —CH₂—O—NH—, —CH₂—CH₂, —CH═CH—, —CH₂—O—, —CH═N—, or —CH₂—NH—;
Y is —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ or —(CH═CH)ᵢ (wherein i=0, 1, 2 or 3 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₁ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (8) are set forth in Table 8 below.

TABLE 8
Representative Compounds in Formula 8:

| Compound | | | | | | | | | | | | | |
|---|---|---|---|---|---|---|---|---|---|---|---|---|---|
| No. | R₁ | R₂ | R₃ | R₄ | R₅ | R₆ | W | -X- | -Y- | m | n | -U- | -Q |
| 286. | H | H | H | H | H | H | | —CH₂O— | —OCH₂— | 1 | 1 | 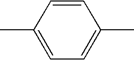 | 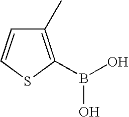 |
| 287. | H | H | | | | | | —CO—O— | —O—CO— | 0 | 0 | 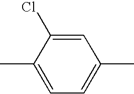 | 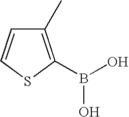 |
| 288. | H | H | | | | | | —CO—O— | —CO—O— | 0 | 0 | 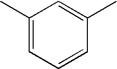 | 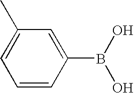 |
| 289. | H | H | | | | | | —CO—NH— | —CO—NH— | 0 | 0 | —CH₂— | 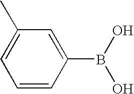 |
| 290. | H | H | | | | | | —CO—NH— | —CO—NH— | 0 | 0 | —CH₂— | 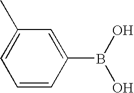 |
| 291. | H | H | | | | | | —CO—NH— | —CO—NH— | 0 | 0 | 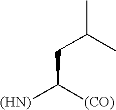 | 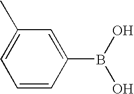 |
| 292. | H | H | | | | | | —CO—NH— | —CO—NH— | 0 | 0 | 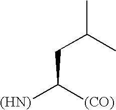 | 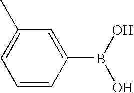 |
| 293. | H | H | H | H | H | H | O | —CH₂O— | —OCH₂— | 1 | 1 | 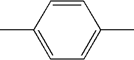 | 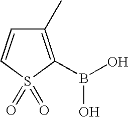 |
| 294. | H | H | | | | | O | —CO—O— | —O—CO— | 0 | 0 | 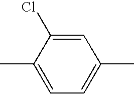 | 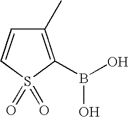 |
| 295. | H | H | | | | | O | —CO—O— | —CO—O— | 0 | 0 | 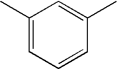 | 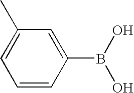 |
| 296. | H | H | | | | | O | —CO—NH— | —CO—NH— | 0 | 0 | —CH₂— | 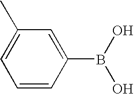 |
| 297. | H | H | | | | | O | —CO—NH— | —CO—NH— | 0 | 0 | —CH₂— | 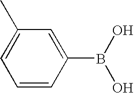 |
| 298. | H | H | | | | | O | —CO—NH— | —CO—NH— | 0 | 0 | 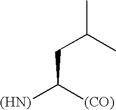 | 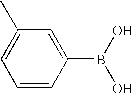 |
| 299. | H | H | | | | | O | —CO—NH— | —CO—NH— | 0 | 0 | 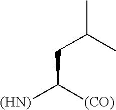 | 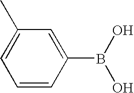 |

The most preferred compounds in Formula (8) are:

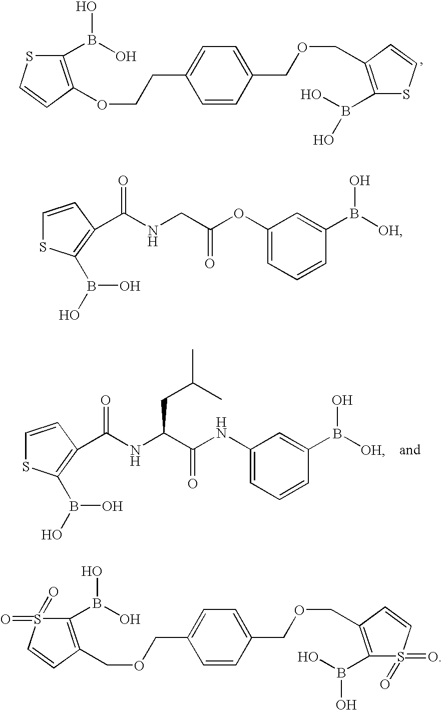

### 9. Ninth Preferred Embodiment

In a ninth preferred embodiment, the compounds are described by formula (9): 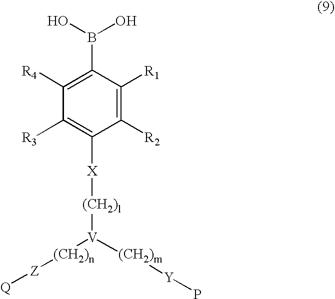
wherein R₁ through R₄, l, m, n, P, Q, V, W. X, Y, and Z can vary in order to optimize affinity, activity, absorption, distribution, metabolism, excretion, pharmacokinetic, toxicological and other properties required for their use as orally deliverable pharmaceuticals.

### a. Preferred Embodiment 9-a

In a more preferred ninth embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m and n is each independently 0, 1 or 2;
X, Y and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene which may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₁ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene which may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 9-b

In another more preferred ninth embodiment,
R₁ through R₄ each independently represents hydrogen, C₁₋₁ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, —SO₂NH₂;
l, m, and n is each independently 0, 1, or 2;
X, Y, and Z is each independently —CO—O—, —O—CO—, —CO—NH—, —NH—CO—, —CH═CH—, —NH—CH₂—, —CH═N—, —CH₂—NH—, —SO₂—O—, —O—SO₂—, —SO₂—NH—, —NH—SO₂— or —N═N—;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

### C. Preferred Embodiment 9-c

In another more preferred ninth embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m and n is each independently 0, 1 or 2;
X, Y and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene which may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene which may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
with the proviso that,
when P or Q is 3-boronophenyl, Y or Z is not —CO—NH or —SO₂—NH—.

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### d. Preferred Embodiment 9-d

In another more preferred ninth embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, —SO₂NH₂;
l, m, and n is each independently 0, 1, or 2;
X, Y, and Z is each independently —CO—O—, —O—CO—, —CO—NH—, —NH—CO—, —CH═CH—, —NH—CH₂—, —CH═N—, —CH₂—NH—, —SO₂—O—, —O—SO₂—, —SO₂—NH—, —NH—SO₂— or —N═N—;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
with the proviso that,
when P or Q is 3-boronophenyl, Y or Z is not —CO—NH or —SO₂—NH—.

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (9) are set forth in Table 9 below.

TABLE 9
Representative Compounds in Formula (9):

| Compound | | | | | | | | | | |
|---|---|---|---|---|---|---|---|---|---|---|
| No. | R₁ | R₂ | R₃ | R₄ | -X- | -Y- | -Z- | l | m | n |
| 300. | H | H | H | H | —CO—O— | —O—CO— | —O—CO— | 0 | 0 | 0 |
| 301. | H | H | H | H | —CO—O— | —O—CO— | —O—CO— | 0 | 0 | 0 |
| 302. | H | H | H | H | —CO—O— | —O—CO— | —O—CO— | 2 | 2 | 2 |
| 303. | H | H | H | H | —CH₂O— | —OCH₂— | —OCH₂— | 0 | 0 | 0 |
| 304. | H | H | H | H | —CO—O— | —NH—CO— | —CO—O— | 0 | 0 | 0 |
| 305. | H | H | H | H | —O—CO— | —NH—CO— | —CO—O— | 0 | 0 | 0 |
| 306. | H | H | H | H | —O—CO— | —NH—CO— | —CO—O— | 0 | 0 | 0 |
| 307. | H | H | H | H | —CO—O— | —CO—NH— | —CO—O— | 0 | 1 | 0 |
| 308. | H | H | H | H | —CO—O— | —CO—NH— | —CO—O— | 0 | 1 | 0 |

| Compound No. | -P | -Q | 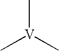 |
|---|---|---|---|
| 300. | 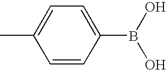 | 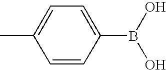 | 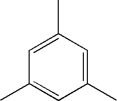 |
| 301. | 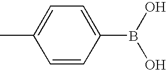 | 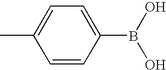 | 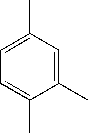 |
| 302. | 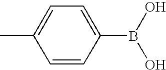 | 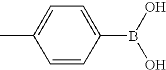 | 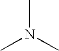 |
| 303. | 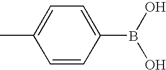 | 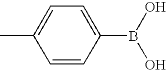 | 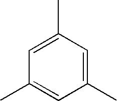 |
| 304. | 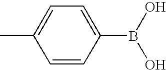 | 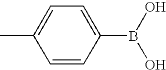 | 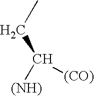 |
| 305. | 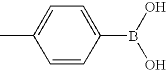 | 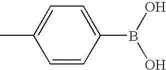 | 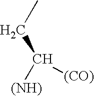 |
| 306. | 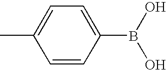 | 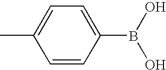 | 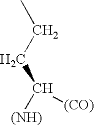 |
| 307. | 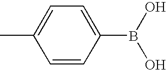 | 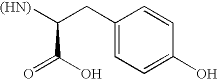 | 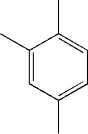 |
| 308. | 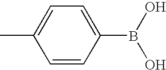 | 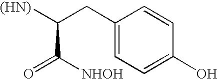 | 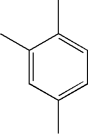 |

The most preferred compounds in Formula (9) are:

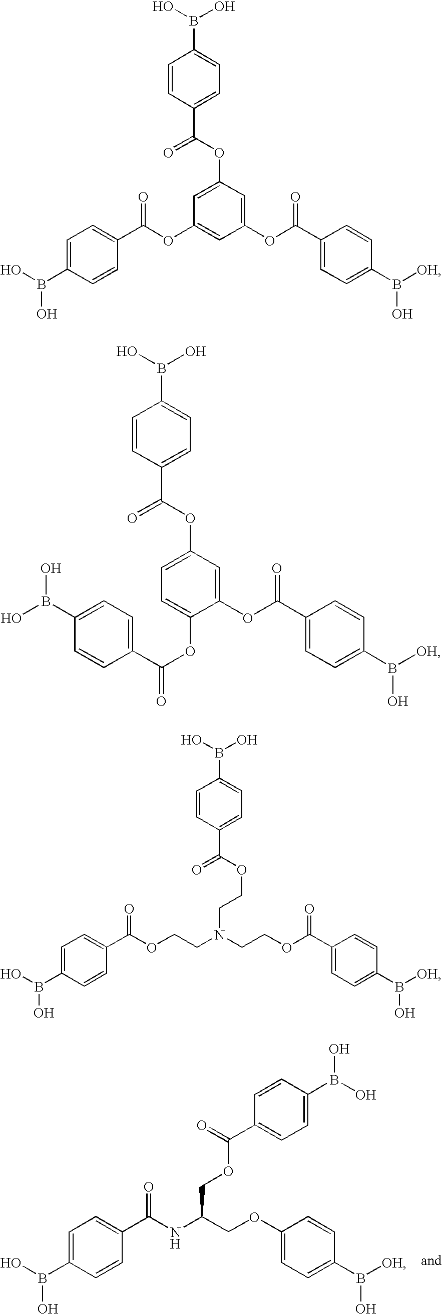

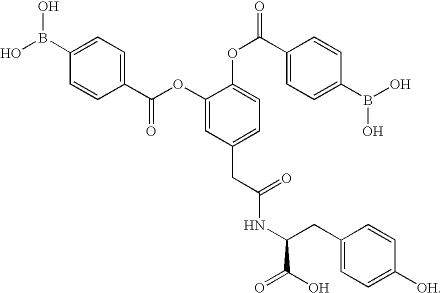

### 10. Tenth Preferred Embodiment

In a tenth preferred embodiment, the compounds are described by formula (10): 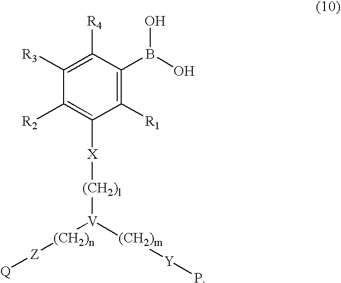
wherein R₁ through R₄, l, m, n, P, Q, V, W, X, Y, and Z can vary in order to optimize affinity, activity, absorption, distribution, metabolism, excretion, pharmacokinetic, toxicological and other properties required for their use as orally deliverable pharmaceuticals.

### a. Preferred Embodiment 10-a

In a more preferred tenth embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m and n is each independently 0, 1 or 2;
X, Y and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene which may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene which may be substituted with one or more substituents R₁₃, and each R₁₃ independently represents hydrogen, C₁₋₆ alkyl, C₃₋₇ cycloalkyl, C₁₋₁ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 10-b

In another more preferred tenth embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —CH═N—, —SO₂—O—, —O—SO₂—, —SO₂—NH—, —NH—SO₂— or —N═N—. Y or Z is not —CO—NH or —SO₂—NH—;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₆₋₄ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO— or —CH₂—).

In this particular embodiment, each heterocyclic alkene is independently a cyclic alkene as defined above, wherein one, two, or all three rings contain(s) one or more S, N or O atom(s).

### c. Preferred Embodiment 10-c

In another more preferred tenth embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m and n is each independently 0, 1 or 2;
X, Y and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene which may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene which may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
with the proviso that,
when R₁ through R₄ represent hydrogen, X is not —NH—CO— or —NH—SO₂—.

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### d. Preferred Embodiment 10-d

In another more preferred tenth embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —CH═N—, —SO₂—O—, —O—SO₂—, —SO₂—NH—, —NH—SO₂— or —N═N—. Y or Z is not —CO—NH or —SO₂—NH—;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
with the proviso that,
when R₁ through R₄ represent hydrogen, X is not —NH—CO— or —NH—SO₂—.

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO— or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (10) are set forth in Table 10 below.

TABLE 10
Representative Compounds in Formula (10):

| Compound | | | | | | | | | | |
|---|---|---|---|---|---|---|---|---|---|---|
| No. | R₁ | R₂ | R₃ | R₄ | -X- | -Y- | -Z- | l | m | n |
| 309. | H | H | H | H | —CO—O— | —O—CO— | —O—CO— | 0 | 0 | 0 |
| 310. | H | H | H | H | —CO—O— | —O—CO— | —O—CO— | 0 | 0 | 0 |
| 311. | H | H | H | H | —CO—O— | —O—CO— | —O—CO— | 2 | 2 | 2 |
| 312. | H | H | H | H | —CH₂O— | —OCH₂— | —OCH₂— | 0 | 0 | 0 |
| 313. | H | H | H | H | —CO—O— | —NH—CO— | —CO—O— | 0 | 0 | 0 |
| 314. | H | H | H | H | —O—CO— | —NH—CO— | —CO—O— | 0 | 0 | 0 |
| 315. | H | H | H | H | —O—CO— | —NH—CO— | —CO—O— | 0 | 0 | 0 |
| 316. | H | H | H | H | —CO—O— | —O—CO— | —CO—O— | 0 | 1 | 0 |
| 317. | H | H | H | H | —CO—O— | —O—CO— | —CO—O— | 0 | 1 | 0 |
| 318. | H | H | —NO₂ | H | —CO—O— | —O—CO— | —O—CO— | 0 | 0 | 0 |
| 319. | H | H | —NO₂ | H | —CO—O— | —O—CO— | —O—CO— | 0 | 0 | 0 |
| 320. | H | H | —NO₂ | H | —CO—O— | —O—CO— | —O—CO— | 2 | 2 | 2 |
| 321. | H | H | —NO₂ | H | —CH₂O— | —OCH₂— | —OCH₂— | 0 | 0 | 0 |
| 322. | H | H | —NO₂ | H | —CO—O— | —NH—CO— | —CO—O— | 0 | 0 | 0 |
| 323. | H | H | —NO₂ | H | —O—CO— | —NH—CO— | —CO—O— | 0 | 0 | 0 |
| 324. | H | H | —NO₂ | H | —O—CO— | —NH—CO— | —CO—O— | 0 | 0 | 0 |
| 325. | H | H | —NO₂ | H | —CO—O— | —O—CO— | —CO—O— | 0 | 1 | 0 |
| 326. | H | H | —NO₂ | H | —CO—O— | —O—CO— | —CO—O— | 0 | 1 | 0 |

| Compound No. | -P | -Q | 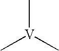 |
|---|---|---|---|
| 309. | 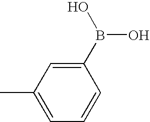 | 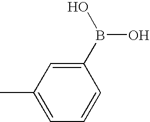 | 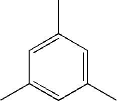 |
| 310. | 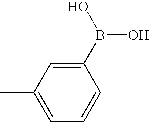 | 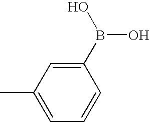 | 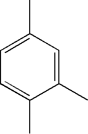 |
| 311. | 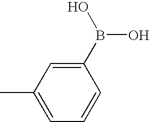 | 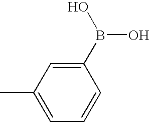 | 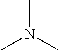 |
| 312. | 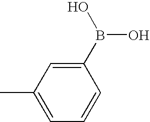 | 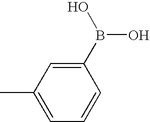 | 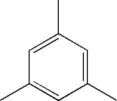 |
| 313. | 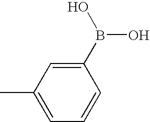 | 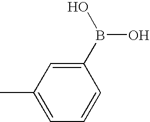 | 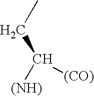 |
| 314. | 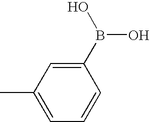 | 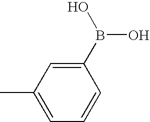 | 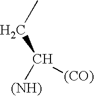 |
| 315. | 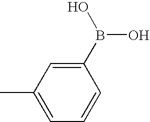 | 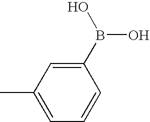 | 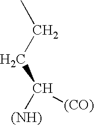 |
| 316. | 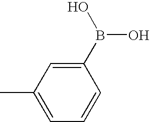 | 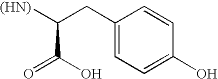 | 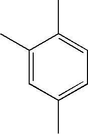 |
| 317. | 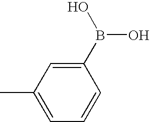 | 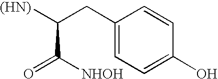 | 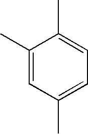 |
| 318. | 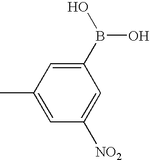 | 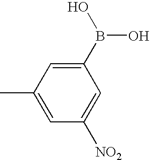 | 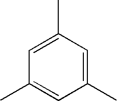 |
| 319. | 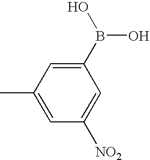 | 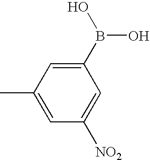 | 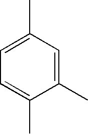 |
| 320. | 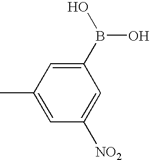 | 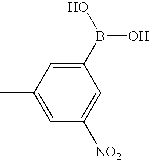 | 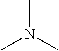 |
| 321. | 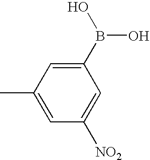 | 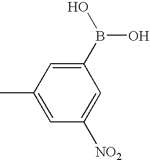 | 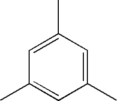 |
| 322. | 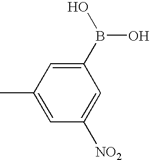 | 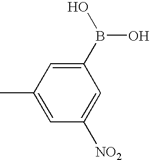 | 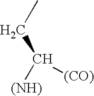 |
| 323. | 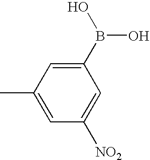 | 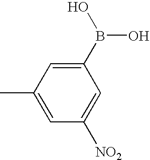 | 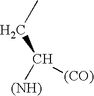 |
| 324. | 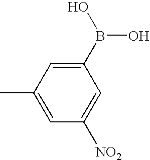 | 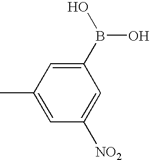 | 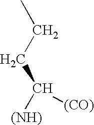 |
| 325. | 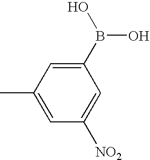 | 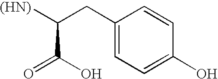 | 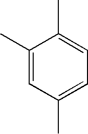 |
| 326. | 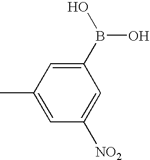 | 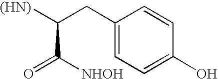 | 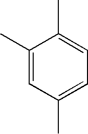 |

Most preferred compounds in Formula (10) are:

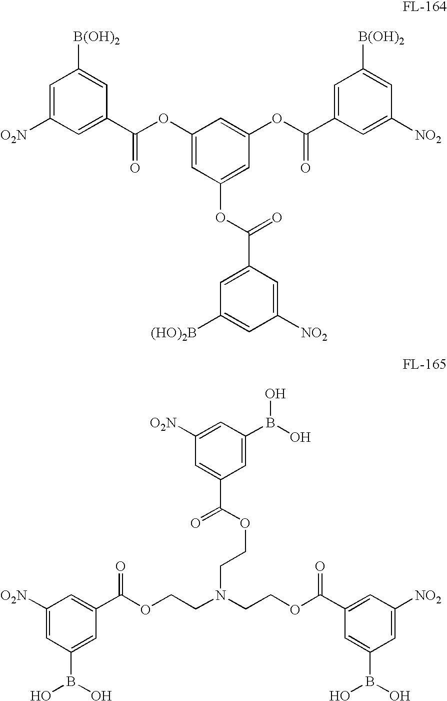

Additional compounds in Formula (10) are:

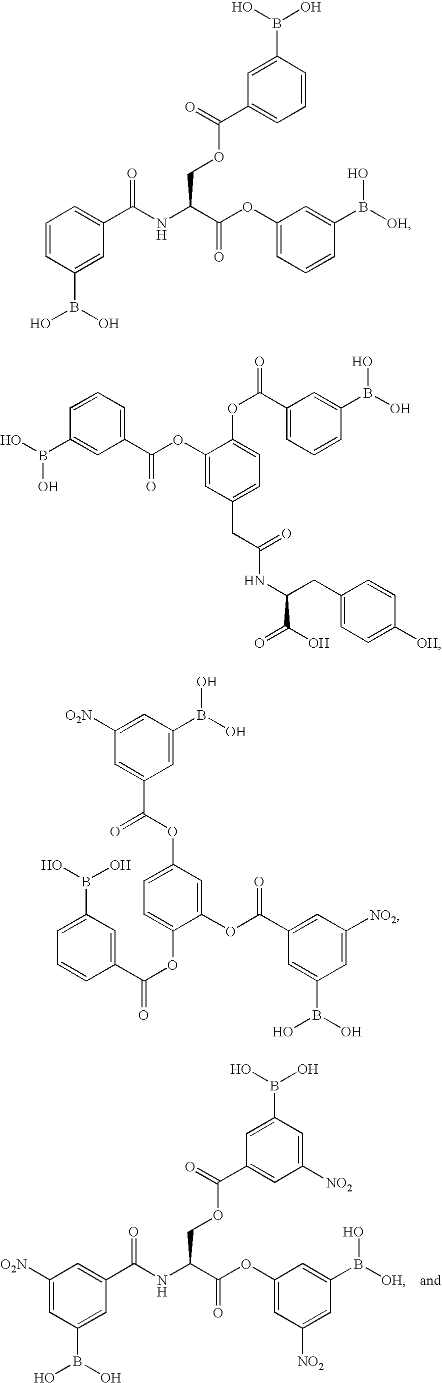

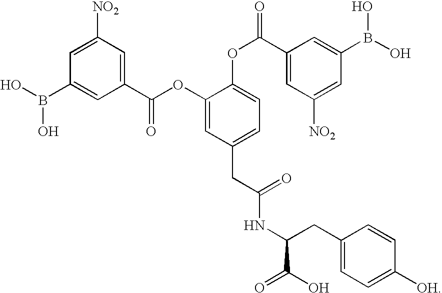

### 11. Eleventh Preferred Embodiment

In an eleventh preferred embodiment, the compounds are described by formula (11): 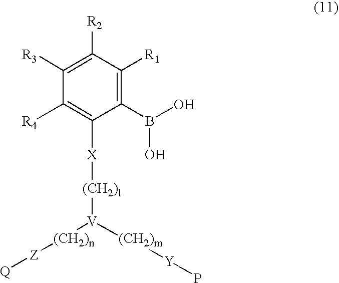
wherein R₁ through R₄, l, m, n, P, Q, V, W, X, Y, and Z can vary in order to optimize affinity, activity, absorption, distribution, metabolism, excretion, pharmacokinetic, toxicological and other properties required for their use as orally deliverable pharmaceuticals.

### a. Preferred Embodiment 11-a

In a more preferred eleventh embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m and n is each independently 0, 1 or 2;
X, Y and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene which may be substituted with one or more substituents R₁₀, and each R₁₀ independently represents hydrogen, C₁₋₆ alkyl, C₃₋₇ cycloalkyl, C₆₋₄ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene which may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 11-b

In another more preferred eleventh embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —CH═N—, —SO₂—O—, —O—SO₂—, —SO₂—NH—, —NH—SO₂— or —N═N—;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₁ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂[wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

### c. Preferred Embodiment 11-c

In another more preferred eleventh embodiment,
R₁ through R₄ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m and n is each independently 0, 1 or 2;
X, Y and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene which may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene which may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
with the proviso that,
(1) when P or Q is 3-boronophenyl, Y or Z is not —CO—NH or —SO₂—NH—; and
(2) when R₁ through R₄ are H, X=—CH₂—NH—, l=m=n=2, V is a nitrogen, Y=Z=—NH—CH₂—, P and Q is not 2-boronophenyl at the same time.

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### d. Preferred Embodiment 11-d

In another more preferred eleventh embodiment,
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —CH═N—, —SO₂—O—, —O—SO₂—, —SO₂—NH—, —NH—SO₂— or —N═N—;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
with the proviso that,
(1) when P or Q is 3-boronophenyl, Y or Z is not —CO—NH or —SO₂—NH—; and
(2) when R₁ through R₄ are H, X=—CH₂—NH—, l=m=n=2, V is a nitrogen, Y=Z=—NH—CH₂—, P and Q is not 2-boronophenyl at the same time.

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (11) are set forth in Table 11 below.

TABLE 11
Representative Compounds in Formula (11):

| Compound | | | | | | | | | | |
|---|---|---|---|---|---|---|---|---|---|---|
| No. | R₁ | R₂ | R₃ | R₄ | -X- | -Y- | -Z- | l | m | n |
| 327. | H | H | H | H | —CO—O— | —O—CO— | —O—CO— | 0 | 0 | 0 |
| 328. | H | H | H | H | —CO—O— | —O—CO— | —O—CO— | 0 | 0 | 0 |
| 329. | H | H | H | H | —CO—O— | —O—CO— | —O—CO— | 2 | 2 | 2 |
| 330. | H | H | H | H | —CH₂O— | —OCH₂— | —OCH₂— | 0 | 0 | 0 |
| 331. | H | H | H | H | —CO—O— | —NH—CO— | —CO—O— | 0 | 0 | 0 |
| 332. | H | H | H | H | —O—CO— | —NH—CO— | —CO—O— | 0 | 0 | 0 |
| 333. | H | H | H | H | —O—CO— | —NH—CO— | —CO—O— | 0 | 0 | 0 |
| 334. | H | H | H | H | —CO—O— | —CO—NH— | —CO—O— | 0 | 1 | 0 |
| 335. | H | H | H | H | —CO—O— | —CO—NH— | —CO—O— | 0 | 1 | 0 |

| Compound No. | -P | -Q | 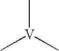 |
|---|---|---|---|
| 327. | 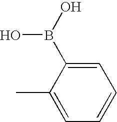 | 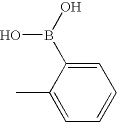 | 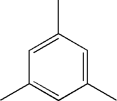 |
| 328. | 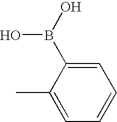 | 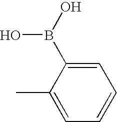 | 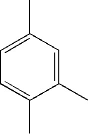 |
| 329. | 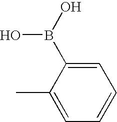 | 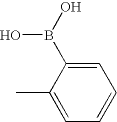 | 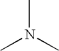 |
| 330. | 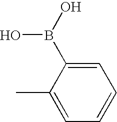 | 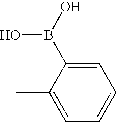 | 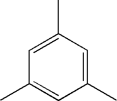 |
| 331. | 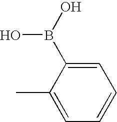 | 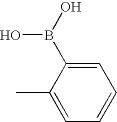 | 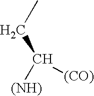 |
| 332. | 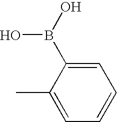 | 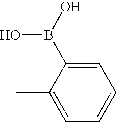 | 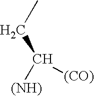 |
| 333. | 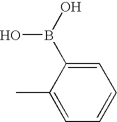 | 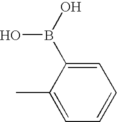 | 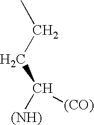 |
| 334. | 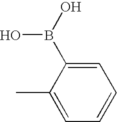 | 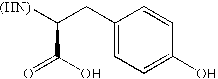 | 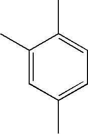 |
| 335. | 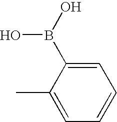 | 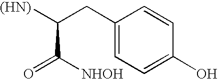 | 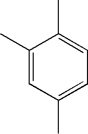 |

The most preferred compounds in Formula (11) are:

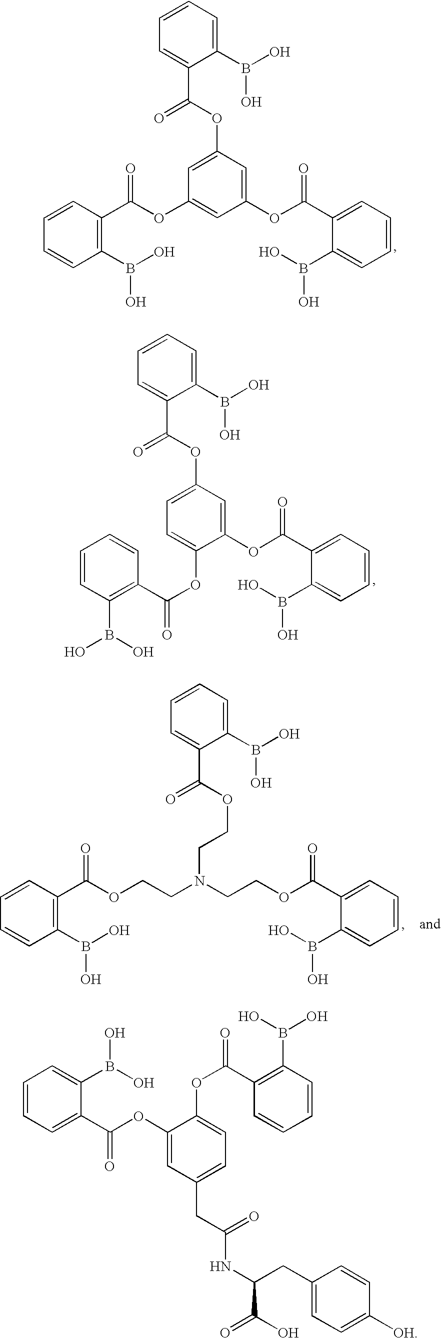

### 12. Twelfth Preferred Embodiment

In a twelfth preferred embodiment, the compounds are described by formula (12): 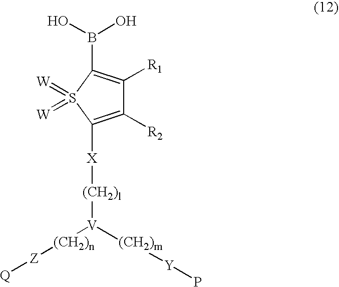
wherein R₁ through R₂, l, m, n, P, Q, V, W, X, Y, and Z can vary in order to optimize affinity, activity, absorption, distribution, metabolism, excretion, pharmacokinetic, toxicological and other properties required for their use as orally deliverable pharmaceuticals.

### a. Preferred Embodiment 12-a

In a more preferred twelfth embodiment,
R₁ and R₂ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m, and n is each independently 0, 1, or 2;
X, Y, and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons.

V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 12-b

In another more preferred twelfth embodiment,
R₁ and R₁ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

### c. Preferred Embodiment 12-c

In another more preferred twelfth embodiment,
R₁ and R₂ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m, and n is each independently 0, 1, or 2;
X, Y, and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons.
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₁ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
with the proviso that,
when P or Q is 3-boronophenyl, Y or Z is not —CO—NH or —SO₂—NH—.

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### d. Preferred Embodiment 12-d

In another more preferred twelfth embodiment,
R₁ and R₁ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₁ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
with the proviso that,
when P or Q is 3-boronophenyl, Y or Z is not —CO—NH or —SO₂—NH—.

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (12) are set forth in Table 12 below.

TABLE 12
Representative Compounds in Formula (12):

| Compound | | | | | | | | | |
|---|---|---|---|---|---|---|---|---|---|
| No. | R₁ | R₂ | W | -X- | -Y- | -Z- | l | m | n |
| 336. | H | H | | —CO—O— | —O—CO— | —O—CO— | 0 | 0 | 0 |
| 337. | H | H | | —CO—O— | —O—CO— | —O—CO— | 0 | 0 | 0 |
| 338. | H | H | | —CO—O— | —O—CO— | —O—CO— | 2 | 2 | 2 |
| 339. | H | H | | —CH₂O— | —OCH₂— | —OCH₂— | 0 | 0 | 0 |
| 340. | H | H | | —CO—O— | —NH—CO— | —CO—O— | 0 | 0 | 0 |
| 341. | H | H | | —O—CO— | —NH—CO— | —CO—O— | 0 | 0 | 0 |
| 342. | H | H | | —O—CO— | —NH—CO— | —CO—O— | 0 | 0 | 0 |
| 343. | H | H | | —CO—O— | —CO—NH— | —CO—O— | 0 | 1 | 0 |
| 344. | H | H | | —CO—O— | —CO—NH— | —CO—O— | 0 | 1 | 0 |
| 345. | H | H | O | —CO—O— | —CO—NH— | —O—CO— | 0 | 0 | 0 |
| 346. | H | H | O | —CO—O— | —O—CO— | —O—CO— | 0 | 0 | 0 |
| 347. | H | H | O | —CO—O— | —O—CO— | —O—CO— | 2 | 2 | 2 |
| 348. | H | H | O | —CH₂O— | —OCH₂— | —OCH₂— | 0 | 0 | 0 |
| 349. | H | H | O | —CO—O— | —NH—CO— | —CO—O— | 0 | 0 | 0 |
| 350. | H | H | O | —O—CO— | —NH—CO— | —CO—O— | 0 | 0 | 0 |
| 351. | H | H | O | —O—CO— | —NH—CO— | —CO—O— | 0 | 0 | 0 |
| 352. | H | H | O | —CO—O— | —CO—NH— | —CO—O— | 0 | 1 | 0 |
| 353. | H | H | O | —CO—O— | —CO—NH— | —CO—O— | 0 | 1 | 0 |

| Compound No. | -P | -Q | 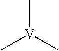 |
|---|---|---|---|
| 336. | 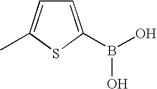 | 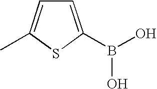 | 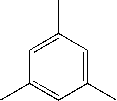 |
| 337. | 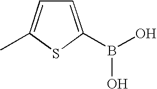 | 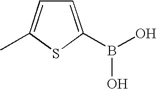 | 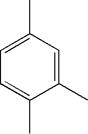 |
| 338. | 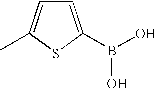 | 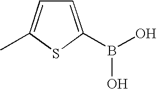 | 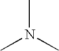 |
| 339. | 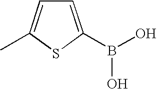 | 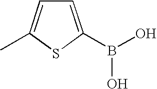 | 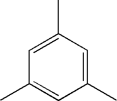 |
| 340. | 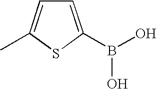 | 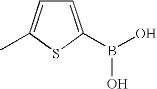 | 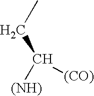 |
| 341. | 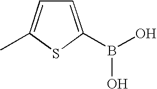 | 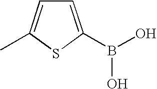 | 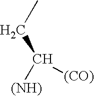 |
| 342. | 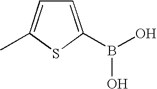 | 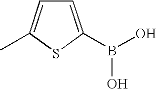 | 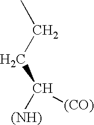 |
| 343. | 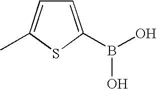 | 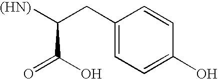 | 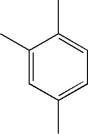 |
| 344. | 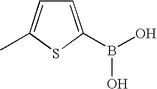 | 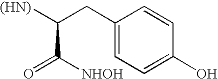 | 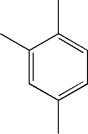 |
| 345. | 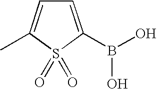 | 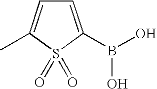 | 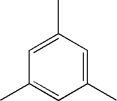 |
| 346. | 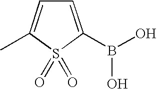 | 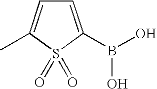 | 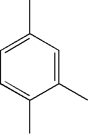 |
| 347. | 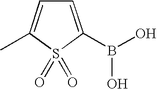 | 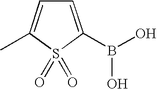 | 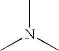 |
| 348. | 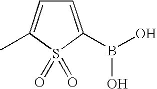 | 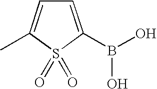 | 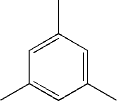 |
| 349. | 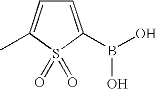 | 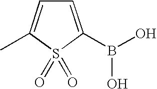 | 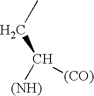 |
| 350. | 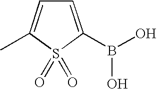 | 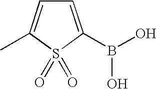 | 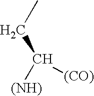 |
| 351. | 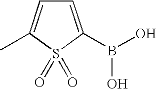 | 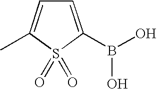 | 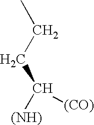 |
| 352. | 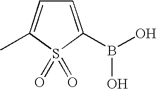 | 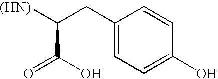 | 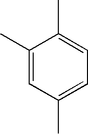 |
| 353. | 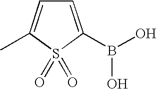 | 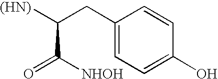 | 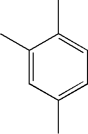 |

The most preferred compounds in Formula (12) are:

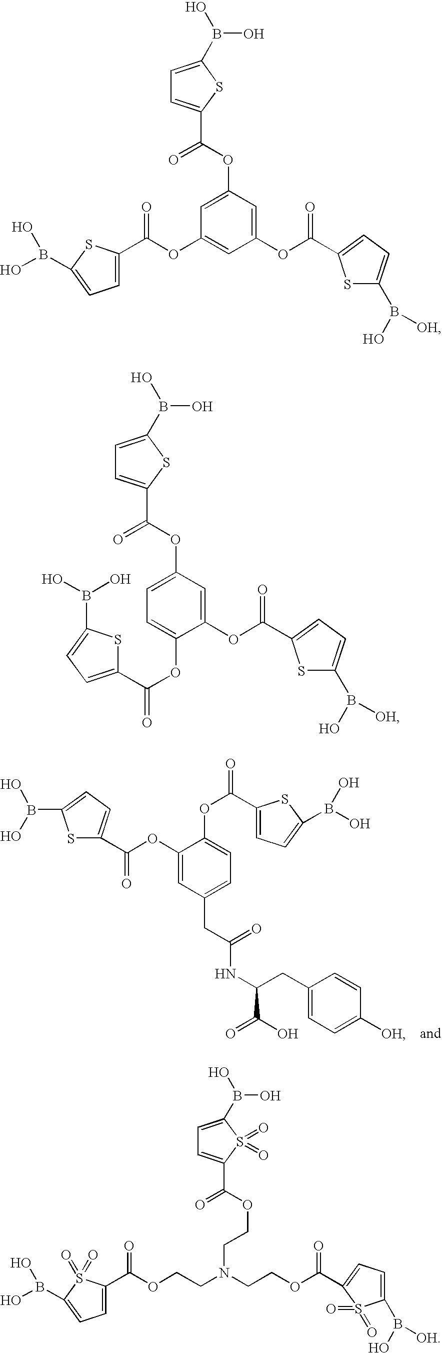

### 13. Thirteenth Preferred Embodiment

In a thirteenth preferred embodiment, the compounds are described by formula (13): 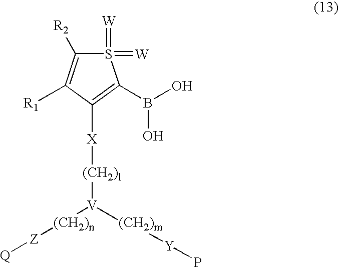
wherein R₁ through R₂, l, m, n, P, Q, V, W, X, Y, and Z can vary in order to optimize affinity, activity, absorption, distribution, metabolism, excretion, pharmacokinetic, toxicological and other properties required for their use as orally deliverable pharmaceuticals.

### a. Preferred Embodiment 13-a

In a more preferred thirteenth embodiment,
R₁ and R₂ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m, and n is each independently 0, 1, or 2;
X, Y, and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons.

V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CHCHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 13-b

In another more preferred thirteenth embodiment,
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

### c. Preferred Embodiment 13-c

In another more preferred thirteenth embodiment,
R₁ and R₂ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m, and n is each independently 0, 1, or 2;
X, Y, and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons.

V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
with the proviso that,
when P or Q is 3-boronophenyl, Y or Z is not —CO—NH or —SO₂—NH—.

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### d. Preferred Embodiment 13-d

In another more preferred thirteenth embodiment,
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R¹⁵R¹⁶N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
with the proviso that,
when P or Q is 3-boronophenyl, Y or Z is not —CO—NH or —SO₂—NH—.

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (13) are set forth in Table 13 below.

TABLE 13
Representative Compounds in Formula (13):

| Compound | | | | | | | | | |
|---|---|---|---|---|---|---|---|---|---|
| No. | R₁ | R₂ | W | -X- | -Y- | -Z- | l | m | n |
| 354. | H | H | | —CO—O— | —O—CO— | —O—CO— | 0 | 0 | 0 |
| 355. | H | H | | —CO—O— | —O—CO— | —O—CO— | 0 | 0 | 0 |
| 356. | H | H | | —CO—O— | —O—CO— | —O—CO— | 2 | 2 | 2 |
| 357. | H | H | | —CH₂O— | —OCH₂— | —OCH₂— | 0 | 0 | 0 |
| 358. | H | H | | —CO—O— | —NH—CO— | —CO—O— | 0 | 0 | 0 |
| 359. | H | H | | —O—CO— | —NH—CO— | —CO—O— | 0 | 0 | 0 |
| 360. | H | H | | —O—CO— | —NH—CO— | —CO—O— | 0 | 0 | 0 |
| 361. | H | H | | —CO—O— | —CO—NH— | —CO—O— | 0 | 1 | 0 |
| 362. | H | H | | —CO—O— | —CO—NH— | —CO—O— | 0 | 1 | 0 |
| 363. | H | H | O | —CO—O— | —CO—NH— | —O—CO— | 0 | 0 | 0 |
| 364. | H | H | O | —CO—O— | —O—CO— | —O—CO— | 0 | 0 | 0 |
| 365. | H | H | O | —CO—O— | —O—CO— | —O—CO— | 2 | 2 | 2 |
| 366. | H | H | O | —CH₂O— | —OCH₂— | —OCH₂— | 0 | 0 | 0 |
| 367. | H | H | O | —CO—O— | —NH—CO— | —CO—O— | 0 | 0 | 0 |
| 368. | H | H | O | —O—CO— | —NH—CO— | —CO—O— | 0 | 0 | 0 |
| 369. | H | H | O | —O—CO— | —NH—CO— | —CO—O— | 0 | 0 | 0 |
| 370. | H | H | O | —CO—O— | —CO—NH— | —CO—O— | 0 | 1 | 0 |
| 371. | H | H | O | —CO—O— | —CO—NH— | —CO—O— | 0 | 1 | 0 |

| Compound No. | -P | -Q | 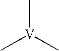 |
|---|---|---|---|
| 354. | 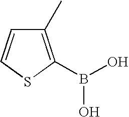 | 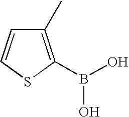 | 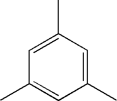 |
| 355. | 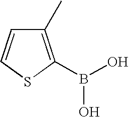 | 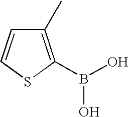 | 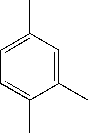 |
| 356. | 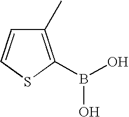 | 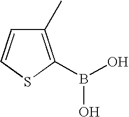 | 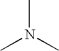 |
| 357. | 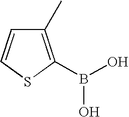 | 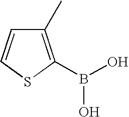 | 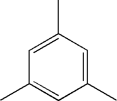 |
| 358. | 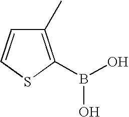 | 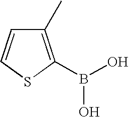 | 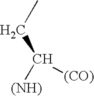 |
| 359. | 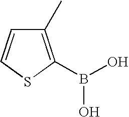 | 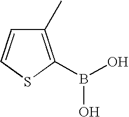 | 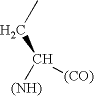 |
| 360. | 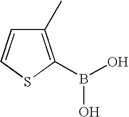 | 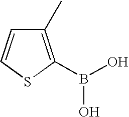 | 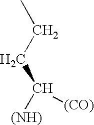 |
| 361. | 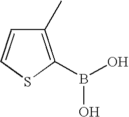 | 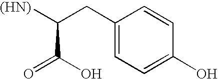 | 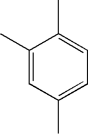 |
| 362. | 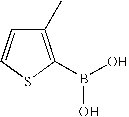 | 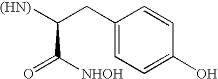 | 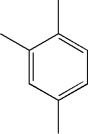 |
| 363. | 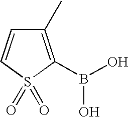 | 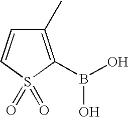 | 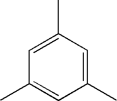 |
| 364. | 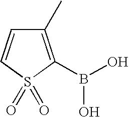 | 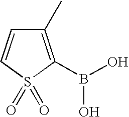 | 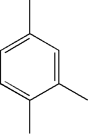 |
| 365. | 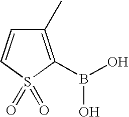 | 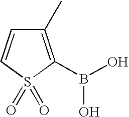 | 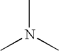 |
| 366. | 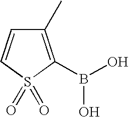 | 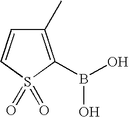 | 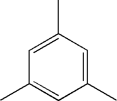 |
| 367. | 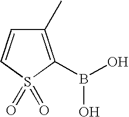 | 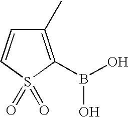 | 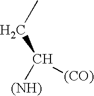 |
| 368. | 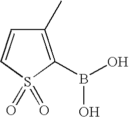 | 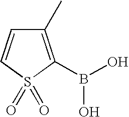 | 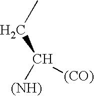 |
| 369. | 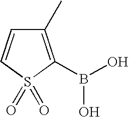 | 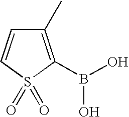 | 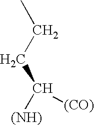 |
| 370. | 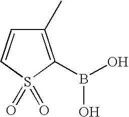 | 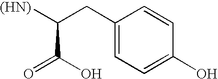 | 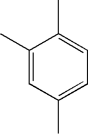 |
| 371. | 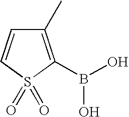 | 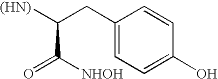 | 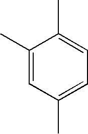 |

The most preferred compounds in Formula (13) are:

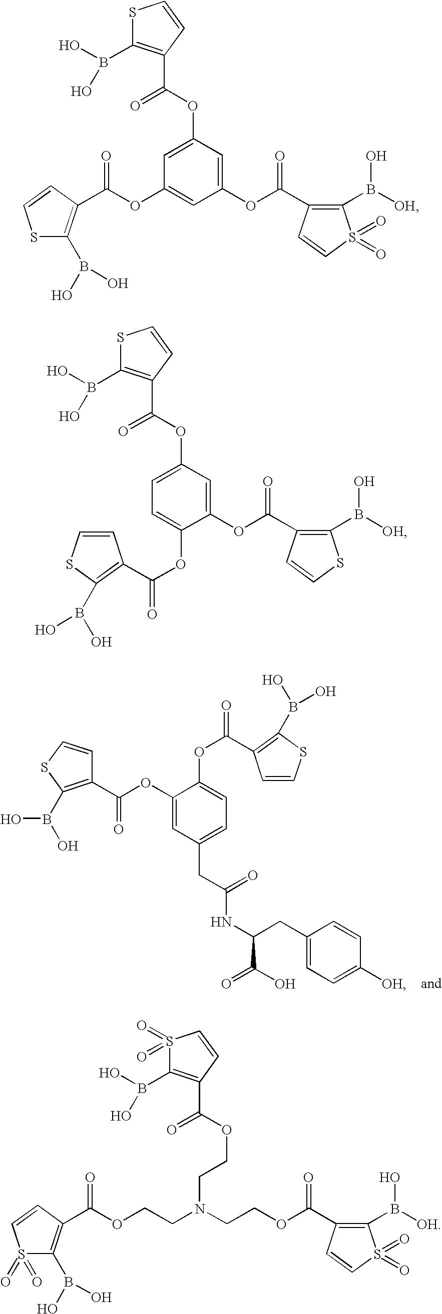

### 14. Fourteenth Preferred Embodiment

In a fourteenth preferred embodiment, the compounds are described by formula (14): 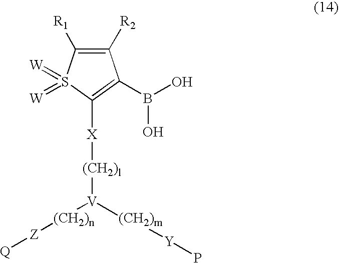
wherein R₁ through R₂, l, m, n, P, Q, V, W, X, Y, and Z can vary in order to optimize affinity, activity, absorption, distribution, metabolism, excretion, pharmacokinetic, toxicological and other properties required for their use as orally deliverable pharmaceuticals.

### a. Preferred Embodiment 14-a

In a more preferred fourteenth embodiment,
R₁ and R₂ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m, and n is each independently 0, 1, or 2;
X, Y, and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons.

V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R¹⁵R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 14-b

In another more preferred fourteenth embodiment,
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R¹⁵R¹⁶N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

### c. Preferred Embodiment 14-c

In another more preferred fourteenth embodiment,
R₁ and R₂ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m, and n is each independently 0, 1, or 2;
X, Y, and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons.

V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₁)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
with the proviso that,
when P or Q is 3-boronophenyl, Y or Z is not —CO—NH or —SO₂—NH—.

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### d. Preferred Embodiment 14-d

In another more preferred fourteenth embodiment,
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
with the proviso that,
when P or Q is 3-boronophenyl, Y or Z is not —CO—NH or —SO₂—NH—.

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (14) are set forth in Table 14 below.

TABLE 14
Representative Compounds in Formula (14):

| Compound | | | | | | | | | |
|---|---|---|---|---|---|---|---|---|---|
| No. | R₁ | R₂ | W | -X- | -Y- | -Z- | l | m | n |
| 372. | H | H | | —CO—O— | —O—CO— | —O—CO— | 0 | 0 | 0 |
| 373. | H | H | | —CO—O— | —O—CO— | —O—CO— | 0 | 0 | 0 |
| 374. | H | H | | —CO—O— | —O—CO— | —O—CO— | 2 | 2 | 2 |
| 375. | H | H | | —CH₂O— | —OCH₂— | —OCH₂— | 0 | 0 | 0 |
| 376. | H | H | | —CO—O— | —NH—CO— | —CO—O— | 0 | 0 | 0 |
| 377. | H | H | | —O—CO— | —NH—CO— | —CO—O— | 0 | 0 | 0 |
| 378. | H | H | | —O—CO— | —NH—CO— | —CO—O— | 0 | 0 | 0 |
| 379. | H | H | | —CO—O— | —CO—NH— | —CO—O— | 0 | 1 | 0 |
| 380. | H | H | | —CO—O— | —CO—NH— | —CO—O— | 0 | 1 | 0 |
| 381. | H | H | O | —CO—O— | —CO—NH— | —O—CO— | 0 | 0 | 0 |
| 382. | H | H | O | —CO—O— | —O—CO— | —O—CO— | 0 | 0 | 0 |
| 383. | H | H | O | —CO—O— | —O—CO— | —O—CO— | 2 | 2 | 2 |
| 384. | H | H | O | —CH₂O— | —OCH₂— | —OCH₂— | 0 | 0 | 0 |
| 385. | H | H | O | —CO—O— | —NH—CO— | —CO—O— | 0 | 0 | 0 |
| 386. | H | H | O | —O—CO— | —NH—CO— | —CO—O— | 0 | 0 | 0 |
| 387. | H | H | O | —O—CO— | —NH—CO— | —CO—O— | 0 | 0 | 0 |
| 388. | H | H | O | —CO—O— | —CO—NH— | —CO—O— | 0 | 1 | 0 |
| 389. | H | H | O | —CO—O— | —CO—NH— | —CO—O— | 0 | 1 | 0 |

| Compound No. | -P | -Q | 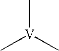 |
|---|---|---|---|
| 372. | 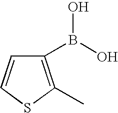 | 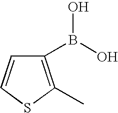 | 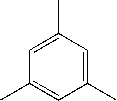 |
| 373. | 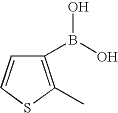 | 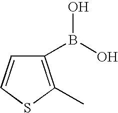 | 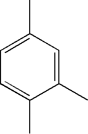 |
| 374. | 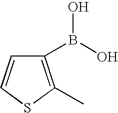 | 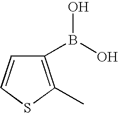 | 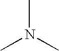 |
| 375. | 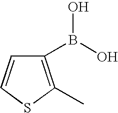 | 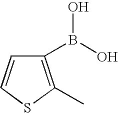 | 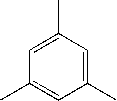 |
| 376. | 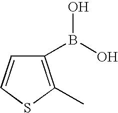 | 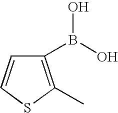 | 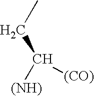 |
| 377. | 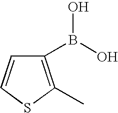 | 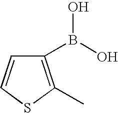 | 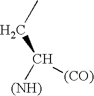 |
| 378. | 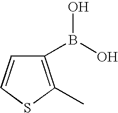 | 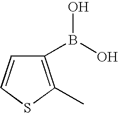 | 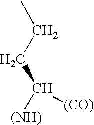 |
| 379. | 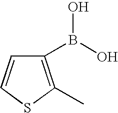 | 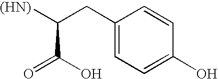 | 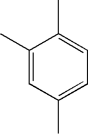 |
| 380. | 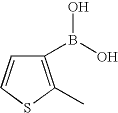 | 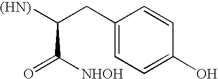 | 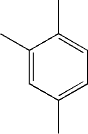 |
| 381. | 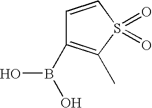 | 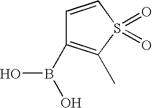 | 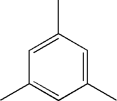 |
| 382. | 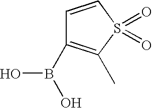 | 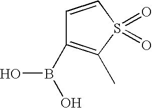 | 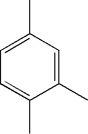 |
| 383. | 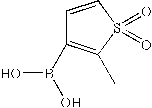 | 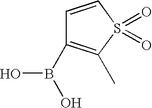 | 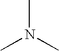 |
| 384. | 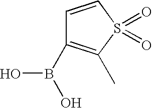 | 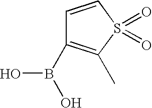 | 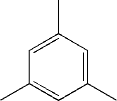 |
| 385. | 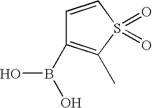 | 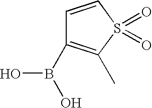 | 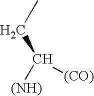 |
| 386. | 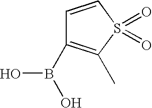 | 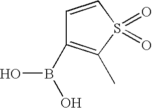 | 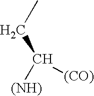 |
| 387. | 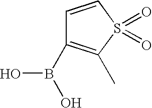 | 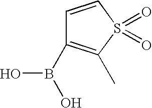 | 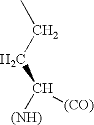 |
| 388. | 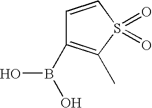 | 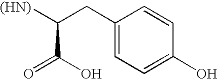 | 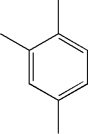 |
| 389. | 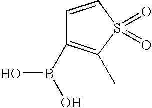 | 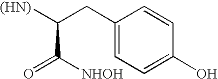 | 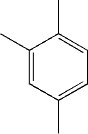 |

The most preferred compounds in Formula (14) are:

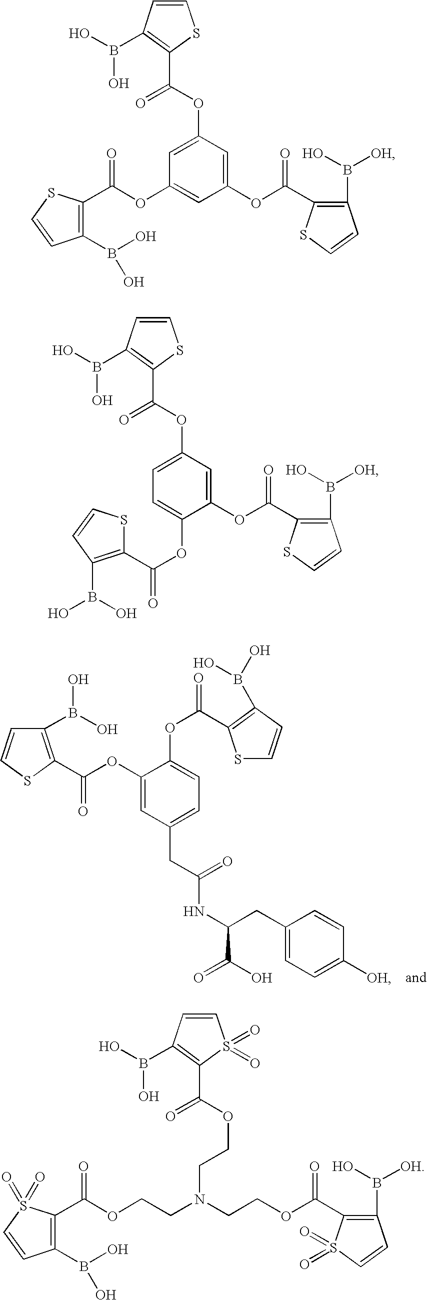

### 15. Fifteenth Preferred Embodiment

In a fifteenth preferred embodiment, the compounds are described by formula (15): 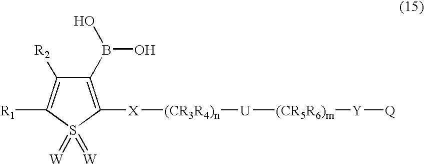
wherein R₁ through R₆, m, n, Q, U, W, X, and Y can vary in order to optimize affinity, activity, absorption, distribution, metabolism, excretion, pharmacokinetic, toxicological and other properties required for their use as orally deliverable pharmaceuticals.

### a. Preferred Embodiment 15-a

In a more preferred fifteenth embodiment,
R₁ and R₂ each independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₃ through R₆ each independently represents hydrogen, C₁₋₁ alkyl, C₃₋₇ cycloalkyl, benzyl or the carbon and attached two Rᵢs, they together form C₃₋₇ cycloalkyl;
m and n is each independently 0 or 1;
X and Y is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5 or 6, and j=0, 1 or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, —CH₂COOH, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₆₋₄ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy (e.g., n-butoxy, i-butoxy, sec-butoxy), R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl).

In this particular embodiment, each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N, O, P or Se atom(s).

### b. Preferred Embodiment 15-b

In another more preferred fifteenth embodiment,
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
R₃ through R₆ each independently represents hydrogen, C₁₋₆ alkyl, C₃₋₇ cycloalkyl, benzyl, or the carbon and attached two Rᵢs, they together form C₃₋₇ cycloalkyl;
m or n is each independently 0 or 1;
X is —CO—O—, —CH₂—O—CO—, —CO—NH—, —CH₂—O—NH—, —CH₂—CH₂, —CH═CH—, —CH₂—O—, —CH═N—, or —CH₂—NH—;
Y is —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ or —(CH═CH)ⱼ (wherein i=0, 1, 2 or 3 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy (e.g., n-butoxy, i-butoxy, sec-butoxy), —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl).

In this particular embodiment, each cyclic alkene is independently a structure containing 1, 2 or 3 rings, each ring containing 5, 6 or 7 carbon atoms and at least one double bond. One, two, or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—).

In this particular embodiment, each heterocyclic alkene is independently a cyclic alkene as defined above, wherein one, two, or all three rings contain(s) one or more S, N or O atom(s).

Non-limiting representative compounds of formula (15) are set forth in Table 15 below.

TABLE 15
Representative Compounds in Formula (15):

| Compound | | | | | | | | | | | | | |
|---|---|---|---|---|---|---|---|---|---|---|---|---|---|
| No. | R₁ | R₂ | R₃ | R₄ | R₅ | R₆ | W | -X- | -Y- | m | n | -U- | -Q |
| 390. | H | H | H | H | H | H | | —CH₂O— | —OCH₂— | 1 | 1 | 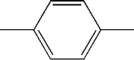 | 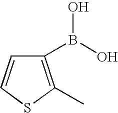 |
| 391. | H | H | | | | | | —CO—O— | —O—CO— | 0 | 0 | 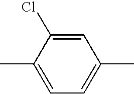 | 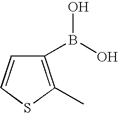 |
| 392. | H | H | | | | | | —CO—O— | —CO—O— | 0 | 0 | 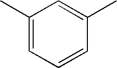 | 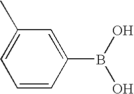 |
| 393. | H | H | | | | | | —CO—NH— | —CO—NH— | 0 | 0 | —CH₂— | 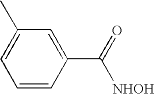 |
| 394. | H | H | | | | | | —CO—NH— | —CO—NH— | 0 | 0 | —CH₂— | 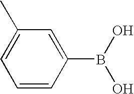 |
| 395. | H | H | | | | | | —CO—NH— | —CO—NH— | 0 | 0 | 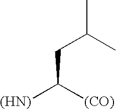 | 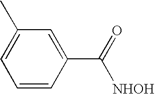 |
| 396. | H | H | | | | | | —CO—NH— | —CO—NH— | 0 | 0 | 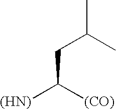 | 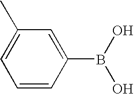 |
| 397. | H | H | H | H | H | H | O | —CH₂O— | —OCH₂— | 1 | 1 | 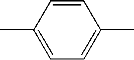 | 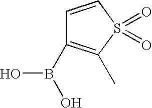 |
| 398. | H | H | | | | | O | —CO—O— | —O—CO— | 0 | 0 | 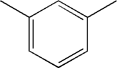 | 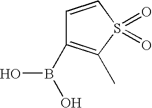 |
| 399. | H | H | | | | | O | —CO—O— | —CO—O— | 0 | 0 | 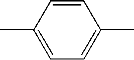 | 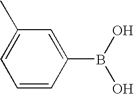 |
| 400. | H | H | | | | | O | —CO—NH— | —CO—NH— | 0 | 0 | —CH₂— | 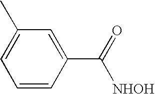 |
| 401. | H | H | | | | | O | —CO—NH— | —CO—NH— | 0 | 0 | —CH₂— | 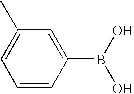 |
| 402. | H | H | | | | | O | —CO—NH— | —CO—NH— | 0 | 0 | 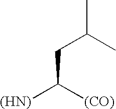 | 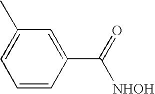 |
| 403. | H | H | | | | | O | —CO—NH— | —CO—NH— | 0 | 0 | 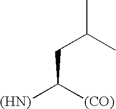 | 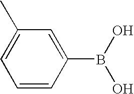 |

The most preferred compounds in Formula (15) are: 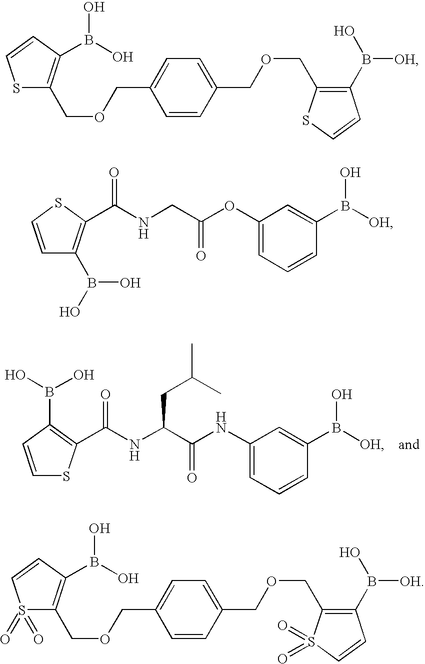
Proteases

The compounds of the present invention can be used to inhibit any coronavirus proteases.

In yet another embodiment, such protease has one or more serine or threonine residues at or near the catalytic site.

In a preferred embodiment, such protease has two or more serine or threonine residues at or near the catalytic site.

In a more preferred embodiment, such protease is a cysteine protease, that is, the catalytic residue of the protease is a cysteine.

In a most preferred embodiment, such protease is a 3CL^{pro}.

Coronaviruses

The boronic acid-based compounds of the present invention can be used to treat infections caused by various viruses and can be used to inhibit protease(s) of various viruses. These various viruses are described in, for example, Fields Virology (4^{th} Edition, Lippincott Williams & Wilkins (2001)).

In one embodiment, the virus belongs to the Coronaviridae family. The Coronaviridae family includes two genera, coronavirus and torovirus, which share many features of genome organization and replication strategy but have different virion morphology and genome lengths. The viral envelopes are studded with long, petal shaped spikes, giving coronaviruses the appearance of a crown (Latin, corona), and the nucleocapsids are long, flexible helices. Other characteristics that define Coronaviridae include the 3′-coterminal, nested-set structure of the mRNAs, unique RNA transcription strategy, genome organization, nucleotide sequence homology, and the properties of their structural proteins.

In one particular embodiment, the virus is a coronavirus. Coronaviruses, a genus in the family Coronaviradae, are large, enveloped, positive-stranded RNA viruses that cause highly prevalent diseases in humans and domestic animals. They have the largest genomes of all RNA viruses and replicate by a unique mechanism, which results in a high frequency of recombination. Virions mature by budding at intracellular membranes, and infection with some coronaviruses induces cell fusion. Coronaviruses were first recognized as a distinct virus group by their characteristic virion morphology in negatively stained preparations. Most coronaviruses can be divided into three serologically distinct groups, although SARS-associated virus does not fit into any of these three known groups. Within each serogroup, the viruses are classified according to their natural hosts, nucleotide sequences, and serologic relationships. Most coronaviruses naturally infect only one animal species or, at most, a limited number of closely related species. Virus replication in vivo can be either disseminated, causing systemic infections, or restricted to a few cell types, often the epithelial cells of the respiratory or enteric tracts and macrophages, causing localized infections. Group I coronaviruses include, for example, HCoV-229E (human respiratory coronavirus), TGEV (porcine transmissible gatroenteritis virus), PRCoV (porcine respiratory coronavirus), CCoV (canine coronavirus), FECoV (Feline enteric coronavirus), FIPV (feline infectious peritonitis virus), and RbCoV (rabbit coronavirus). Group II coronaviruses include, for example, HCoV-OC43 (human respiratory coronavirus), MHV (murine hepatitis virus), SDAV (sialodacryoadenitis virus), HEV (porcine hemagglutinating encephalomyelitis virus), and BCoV (bovine coronavirus). Group III coronaviruses include, for example, IBV (avain infectious bronchitis virus) and TCoV (turkey coronavirus). The SARS-associated coronavirus genome appears to be closer to the murine, bovine, porcine, and human coronaviruses in Group II and avian coronavirus IBV in Group I.

Non-limiting examples of the coronavirus include SARS-associated virus, rat coronavirus (ATCC Nos. VR-1410 (Sialodacryoadenitis virus, deposited as rat coronavirus), and VR-882), murine hepatitis virus (ATCC Nos. VR-1426, VR-246, VR-261, VR-764, VR-765 (murine hepatitis virus deposited as mouse hepatitis virus), and VR-766), human enteric coronavirus (ATCC No. VR-1475), feline coronavirus (ATCC Nos. VR-2004, VR-2009. VR-2125, VR-2126, VR-2127, VR-2128, VR-2201, VR-2202, VR-867, VR-989, and VR-990), canine coronavirus (ATCC Nos. VR-2068 and VR-809), infectious bronchitis virus (ATCC Nos. VR-21, VR-22, VR-817, and VR-841), human coronavirus 229E (ATCC No. VR-740), transmissible gastroenteritis virus (porcine respiratory coronavirus) (ATCC Nos. VR-743 and VR-763), human coronavirus OC43 (ATCC No. VR-759), bovine coronavirus (calf diarrheal coronavirus) (ATCC No. VR-874), rat coronavirus (ATCC No. VR-882); turkey coronavirus (ATCC No. VR-911), rabbit coronavirus (ATCC No. VR-920), and transmissible gastroenteritis virus (ATCC No. VR-2384).

Salts and Derivatives

Various pharmaceutically acceptable salts, ether derivatives, ester derivatives, acid derivatives, and aqueous solubility altering derivatives of the active compound also are encompassed by the present invention. The present invention further includes all individual enantiomers, diastereomers, racemates, and other isomer of the compound. The invention also includes all polymorphs and solvates, such as hydrates and those formed with organic solvents, of this compound. Such isomers, polymorphs, and solvates may be prepared by methods known in the art, such as by regiospecific and/or enantioselective synthesis and resolution, based on the disclosure provided herein.

Suitable salts of the compound include, but are not limited to, acid addition salts, such as those made with hydrochloric, hydrobromic, hydroiodic, perchloric, sulfuric, nitric, phosphoric, acetic, propionic, glycolic, lactic pyruvic, malonic, succinic, maleic, fumaric, malic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid; salts made with saccharin; alkali metal salts, such as sodium and potassium salts; alkaline earth metal salts, such as calcium and magnesium salts; and salts formed with organic or inorganic ligands, such as quaternary ammonium salts.

Additional suitable salts include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate salts of the compound of the present invention.

Prodrugs and active metabolites of compounds disclosed herein are also within the scope of the invention.

A prodrug is a pharmacologically inactive compound that is converted into a pharmacologically active agent by a metabolic transformation. In vivo, a prodrug is acted on by naturally occurring enzyme(s) resulting in liberation of the pharmacologically active agent. Conventional procedures for the selection and preparation of suitable prodrug derivatives are described, for example, in “Design of Prodrugs”, ed. H. Bundgaard, Elsevier, 1985.

An active metabolite is a compound which results from metabolism of another compound after administration of the latter to a subject. Metabolites can be identified by techniques well-known in the art.

Formulation and Administration

Suitable dosage forms include but are not limited to oral, rectal, sub-lingual, mucosal, nasal, ophthalmic, subcutaneous, intramuscular, intravenous, transdermal, spinal, intrathecal, intra-articular, intra-arterial, sub-arachinoid, bronchial, lymphatic, and intra-uterille administration, and other dosage forms for systemic delivery of active ingredients. In a preferred embodiment, the dosage form is suitable for injection.

To prepare such pharmaceutical dosage forms, one or more of the aforementioned compounds of formulae (A), (B) and (1)-(15), are intimately admixed with a pharmaceutical carrier according to conventional pharmaceutical compounding techniques. The carrier may take a wide variety of forms depending on the form of preparation desired for administration.

For parenteral formulations, the carrier will usually comprise sterile water, though other ingredients, for example, ingredients that aid solubility or for preservation, may be included. Injectable solutions may also be prepared in which case appropriate stabilizing agents may be employed.

In preparing the compositions in oral dosage form, any of the usual pharmaceutical media may be employed. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like. Due to their ease in administration, tablets and capsules represent the most advantageous oral dosage unit form. If desired, tablets may be sugar coated or enteric coated by standard techniques.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Treatment methods of the present invention using formulations suitable for oral administration may be presented as discrete units such as capsules, cachets, tablets, or lozenges, each containing a predetermined amount of the active ingredient as a powder or granules. Optionally, a suspension in an aqueous liquor or a non-aqueous liquid may be employed, such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration usually comprise a sterile aqueous preparation of the active compound, which preferably is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery or delivery directly to the CNS. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

Nasal and other mucosal spray formulations (e.g. inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

Formulations for rectal administration may be presented as a suppository with a suitable carrier such as cocoa butter, hydrogenated fats, or hydrogenated fatty carboxylic acids.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, formulations of this invention may further include one or more accessory ingredient(s) selected from diluents, buffers, flavoring agents, binders, disintegrants, surface active agents, thickeners, lubricants, preservatives (including antioxidants), and the like.

The formulation of the present invention can have immediate release, sustained release, delayed-onset release or any other release profile known to one skilled in the art.

The subject is preferably an animal, including, but not limited, to an animal such a cow, horse, sheep, pig, chicken, turkey, quail, cat, dog, mouse, rat, rabbit, guinea pig, etc., and is more preferably a mammal, and most preferably a human.

### EXAMPLES

The following examples illustrate the invention, but are not limiting.

### Example 1

### Synthesis of Select Compounds

1.1 General Procedure A: Synthesis of Boronobenzoic Acid Esters

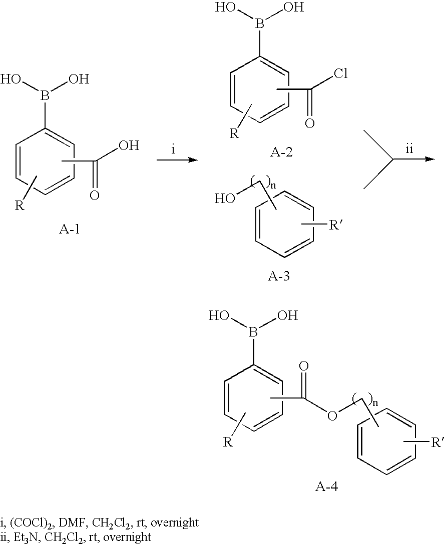

In step (i) of general procedure A, oxalyl chloride (35 μL, 0.4 mmol) was added to a suspension of carboxyl boronic acid derivative A-1 (0.2 mmol), 1 drop of DMF and 5 mL of anhydrous CH₂Cl₂. The reaction mixture was stirred at room temperature overnight and then evaporated to dryness to afford acid chloride A-2 as a yellow solid, which was used without further purification.

In step (ii), a solution of the acid chloride A-2 (0.2 mmol, obtained from step (i) above) in 5 mL of anhydrous CH₂Cl₂ was added dropwise to an ice-cold solution of A-3, anhydrous triethyl amine (42 μL, 0.3 mmol) and 10 mL of anhydrous CH₂Cl₂. The reaction mixture was stirred at room temperature overnight and then evaporated to dryness. It was then dissolved in 25 mL of ethyl acetate and washed with 1N aqueous HCl, 10% aqueous NaHCO₃, saturated brine solution, dried (Na₂SO₄) and then concentrated. The residue was purified by flash chromatography eluting with MeOH-ethyl acetate (1:10). The product yielded from step (ii) is A4.

### 3-(4-Nitrophenoxycarbonyl)-5-nitrophenylboronic acid (FL-061)

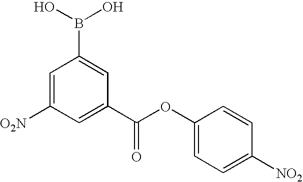

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with p-nitrophenol (21 mg, 0.15 mmol) according to general procedure A, step (ii), to give 42 mg (84% yield) of the desired compound as a white powder, mp: 205-207° C. ¹H-NMR(300 MHz, 5% D₂O in d₆-DMSO): δ 7.61-7.66 [m, 2H, Ar—H], 8.30-8.35 [m, 2H, Ar—H], 8.78-8.89 [m, 3H, Ar—H].

### 3-(2-Nitrophenoxycarbonyl)-5-nitrophenylboronic acid (FL-062)

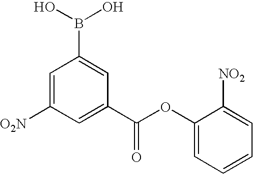

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with o-nitrophenol (21 mg, 0.15 mmol) according to general procedure A, step (ii), to give 36 mg (72% yield) of the desired compound as a pale yellow powder, mp: 128-130° C. ¹H-NMR(300 MHz, 5% D₂O in d₆-DMSO): δ 7.52-7.71 [m, 2H, Ar—H], 7.81-7.94 [m, 1H, Ar—H], δ8.13-8.24 [m, 1H, Ar—H], 8.70-8.93 [m, 3H, Ar—H].

### 3-(3-Nitrophenoxycarbonyl)-5-nitrophenylboronic acid (FL-063)

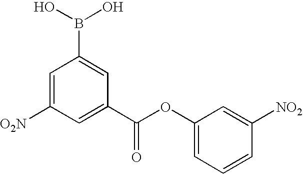

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with m-nitrophenol (21 mg, 0.15 mmol) according to general procedure A, step (ii), to give 41 mg (82% yield) of the desired compound as a light yellow crystal, mp: 185-187C. ¹H-NMR(300 MHz, 5% D₂O in d₆-DMSO): δ7.72-7.81 [m, 2H, Ar—H], δ8.15-8.29 [m, 2H, Ar—H], 8.76-8.89 [m, 3H, Ar—H).

### 3-(4-Methoxyphenoxycarbonyl)-5-nitrophenylboronic acid (FL-064)

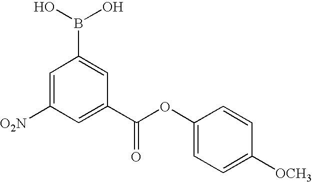

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with p-methoxyphenol (19 mg, 0.15 mmol) according to general procedure A, step (ii), to give 39 mg (81% yield) of the desired compound as a white needle, mp: 67° C. ¹H-NMR(300 MHz, 5% D₂O in d₆-DMSO): δ 3.74 [s, 3H, OCH₃], 6.93-6.99 [m, 2H, Ar—H], 7.19-7.24 [m, 2H, Ar—H], 8.72-8.87 [m, 3H, Ar—H].

### 3-(4-Ethoxycarbonylphenoxycarbonyl)-5-nitrophenylboronic acid (FL-065)

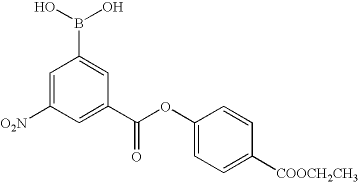

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with ethyl 4-hydroxybenzoate (25 mg, 0.15 mmol) according to general procedure A, step (ii), to give 51 mg (94% yield) of the desired compound as a white powder, mp: 230-232° C. ¹H-NMR(400 MHz, 5% D₂O in d₆-DMSO): δ1.31 [t, 2H, CH₂CH₃], 4.30 [q, 3H, CH₂CH₃], 7.50 [d, 2H, Ar—H], 8.05 [d, 2H, Ar—H], 8.78-8.90 μm, 3H, Ar—H].

### 3-(2-Oxo-benzo[1,3]oxathiol-6-oxycarbonyl)-5-nitrophenylboronic acid (FL-067)

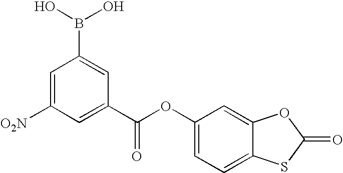

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 6-hydroxy-1,3-benzoxathiol-2-one (25 mg, 0.15 mmol) according to general procedure A, step (ii), to give 50 mg (92% yield) of the desired compound as a white powder, mp: 145-147° C. ¹H-NMR(300 MHz, 5% D₂O in d₆-DMSO): δ7.28-7.36 [m, 1H, Ar—H], 7.56-7.64 [m, 1H, Ar—H], 7.78-7.86 [m, 1H, Ar—H], 8.72-8.86 [m, 3H, Ar—H].

### 3-(2-Cyanophenoxycarbonyl)-5-nitrophenylboronic acid (FL-068)

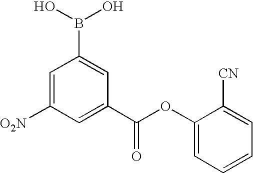

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 2-hydroxybenzonitrile (18 mg, 0.15 mmol) according to general procedure A, step (ii), to give 43 mg (92% yield) of the desired compound as a white powder, mp: 248-250° C. ¹H-NMR(400 MHz, 5% D₂O in d₆-DMSO): δ7.48-7.57 [m, 1H, Ar—H], 7.62-7.69 [m, 1H, Ar—H], 7.78-7.89 [m, 1H, Ar—H], 7.96-8.01 [m, 1H, Ar—H], 8.83-8.94 [m, 3H, Ar—H].

### 3-(2-Chloro-6-nitrophenoxycarbonyl)-5-nitrophenylboronic acid (FL-069)

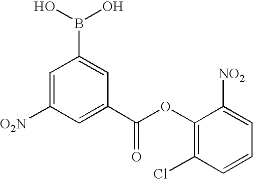

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 2-chloro-6-nitrophenol (26 mg, 0.15 mmol) according to general procedure A, step (ii), to give 39 mg (71% yield) of the desired compound as a yellow needle, mp: 179° C. ¹H-NMR(300 MHz, 5% D₂O in d₆-DMSO): δ7.54-7.72 [m, 1H, Ar—H], 8.01-8.24 [m, 2H, Ar—H], 8.65-8.94 [m, 3H, Ar—H].

### 3-(2-Ethoxycarbonyl-phenoxycarbonyl)-5-nitrophenylboronic acid (FL-071)

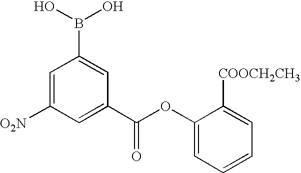

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with ethyl salicylate (25 mg, 0.15 mmol) according to general procedure A, step (ii), to give 35 mg (64% yield) of the desired compound as a yellow semisolid. ¹H-NMR(300 MHz, 5% D₂O in d₆-DMSO): δ1.24 [t, 2H, CH₂CH₃], 4.31 [q, 3H, CH₂CH₃], 7.41-7.57 [m, 2H, Ar—H], 7.81-8.05 [m, 2H, Ar—H], 8.67-8.93 [m, 3H, Ar—H].

### 3-(2-Chlorophenoxycarbonyl)-5-nitrophenylboronic acid (FL-072)

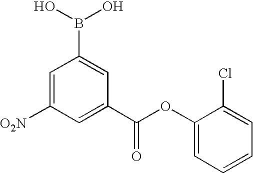

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with o-chlorophenol (19 mg, 0.15 mmol) according to general procedure A, step (ii), to give 44 mg (90% yield) of the desired compound as a white powder, mp: 138-140C. ¹H-NMR(300 MHz, 5% D₂O in d₆-DMSO): δ 7.38-7.78 [m, 4H, Ar—H], 8.71-9.04 [m, 3H, Ar—H].

### 3-(2-Methylphenoxycarbonyl)-5-nitro-phenylboronic acid (FL-073)

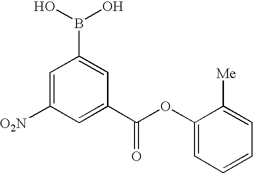

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with o-cresol (16 mg, 0.15 mmol) according to general procedure A, step (ii), to give 40 mg (89% yield) of the desired compound as a yellow powder, mp: 175° C. (dec). ¹H-NMR(300 MHz, 5% D₂O in d₆-DMSO): δ 2.17 [t, 3H, CH₃], 7.21-7.40 [m, 3H, Ar—H], 8.15-8.22 [m, 1H, Ar—H], 8.65-8.91 [m, 3H, Ar—H].

### 3-(2-Methoxylphenoxycarbonyl)-5-nitrophenylboronic acid (FL-074)

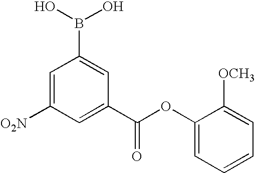

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with guaiacol (19 mg, 0.15 mmol) according to general procedure A, step (ii), to give 44 mg (92% yield) of the desired compound as a yellow semisolid. ¹H-NMR(300 MHz, 5% D₂O in d₆-DMSO): δ3.69[s, 3H, OCH₃], 7.46-8.03 [m, 4H, Ar—H], 8.64-8.97 [m, 3H, Ar—H].

### 3-(3-Methylphenoxycarbonyl)-5-nitrophenylboronic acid (FL-075)

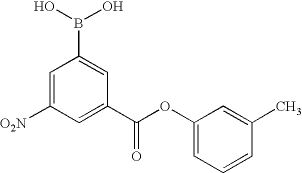

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with m-cresol (16 mg, 0.15 mmol) according to general procedure A, step (ii), to give 43 mg (82% yield) of the desired compound To make compound (1m), was treated according to general procedure A, step (ii), to give 41 mg (91% yield) of the desired compound as a yellow semisolid. ¹H-NMR(300 MHz, 5% D₂O in d₆-DMSO): δ2.35 [t, 3H, CH₃], 7.79-8.30 [m, 4H, Ar—H], 8.71-8.91 [m, 3H, Ar—H].

### 3-(2,6-Dichlorophenoxycarbonyl)-5-nitrophenylboronic acid (FL-080)

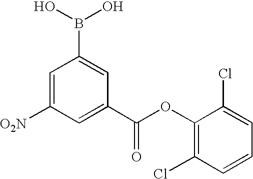

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 2,6-dichlorophenol (25 mg, 0.15 mmol) according to general procedure A, step (ii), to give 43 mg (80% yield) of the desired compound as a pale yellow powder, mp: 167-169° C. ¹H-NMR(300 MHz, 5% D₂O in d₆-DMSO): δ7.39-7.44 [m, 1H, Ar—H], 7.52-7.67 [m, 2H, Ar—H], 8.83 [s, 1H, Ar—H], 8.94-8.96 [m, 2H, Ar—H].

### 3-(2-Fluorophenoxycarbonyl)-5-nitrophenylboronic acid (FL-081)

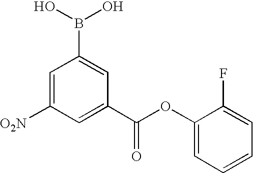

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 2-fluorophenol (17 mg, 0.15 mmol) according to general procedure A, step (ii), to give 39 mg (85% yield) of the desired compound as a white needle, mp: 144-146C. ¹H-NMR (400 MHz, 5% D₂O in d₆-DMSO): δ 7.29-7.53 [m, 4H, Ar—H], 8.80 [s, 1H, Ar—H], 8.89-8.93 [m, 2H, Ar—H].

### 3-(2,6-Difluorophenoxycarbonyl)-5-nitrophenylboronic acid (FL-082)

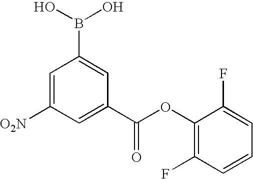

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 2,6-difluorophenol (20 mg, 0.15 mmol) according to general procedure A, step (ii), to give 39 mg (81% yield) of the desired compound as a white powder, mp: 124-126° C. ¹H-NMR(400 MHz, 5% D₂O in d₆-DMSO): δ7.23-7.38 [m, 2H, Ar—H], 7.38-7.48 [m, 1H, Ar—H], 8.81 [s, 1H, Ar—H], 8.86-8.96 [m, 2H, Ar—H].

### 3-(4-Fluorophenoxycarbonyl)-5-nitrophenylboronic acid (FL-085)

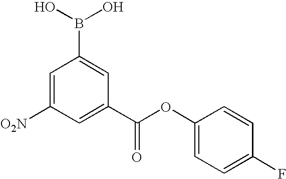

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 4-fluorophenol (17 mg, 0.15 mmol) according to general procedure A, step (ii), to give 41 mg (89% yield) of the desired compound as a white powder, mp: 233-235° C. ¹H-NMR(400 MHz, 5% D₂O in d₆-DMSO): δ 7.21-7.42 [m, 4H, Ar—H], 8.76 [s, 1H, Ar—H], 8.83-8.95 [m, 2H, Ar—H].

### 3-(2,6-Dichlorobenzyloxycarbonyl-5-nitrophenylboronic acid (FL-086)

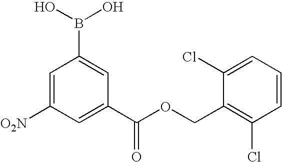

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 2,6-dichlorobenzyl alcohol (27 mg, 0.15 mmol) according to general procedure A, step (ii), to give 49 mg (89% yield) of the desired compound as a pale yellow semisolid. ¹H-NMR(400 MHz, 5% D₂O in d₆-DMSO): δ5.62 [s, 2H, Ar—CH₂], 7.26-7.62 [m, 3H, Ar—H], 8.57-8.95 [m, 3H, Ar—H].

### 3-(4-pyridyloxycarbonyl)-5-nitro-phenylboronic acid (FL-087)

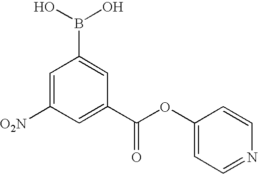

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 4-pyridinol (14 mg, 0.15 mmol) according to general procedure A, step (ii), to give 34 mg (79% yield) of the desired compound as a pale yellow powder, mp: 165° C. (dec). ¹H-NMR(400 MHz, 5% D₂O in d₆-DMSO): δ7.43-7.50 [m, 1H, Ar—H], 8.67-8.72 [m, 1H, Ar—H], 8.77-8.86 [m, 3H, Ar—H], 8.86-8.97 [m, 1H, Ar—H].

### 3-(2-Chloro-3-pyridyloxycarbonyl)-5-nitrophenylboronic acid (FL-089)

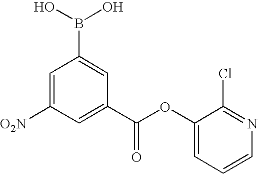

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 2-chloro-3-pyridinol (19 mg, 0.15 mmol) according to general procedure A, step (ii), to give 37 mg (77% yield) of the desired compound as a white powder, mp: 146-148° C. ¹H-NMR(400 MHz, 5% D₂O in d₆-DMSO): δ7.57-7.63 [m, 1H, Ar—H], 8.05-8.09 [m, 1H, Ar—H], 8.39-8.43 [m, 1H, Ar—H], 8.81 [s, 1H, Ar—H], 8.89-8.94 [m, 2H, Ar—H].

### 3-(2-Bromo-1-indanoxycarbonyl)-5-nitrophenylboronic acid (FL-090)

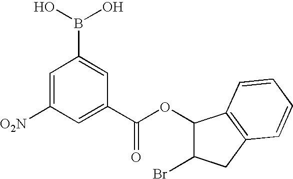

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 2-bromo-1-indanol (32 mg, 0.15 mmol) according to general procedure A, step (ii), to give 43 mg (71% yield) of the desired compound as a white powder, mp: 177-179C. ¹H-NMR(400 MHz, 5% D₂O in d₆-DMSO): δ3.30 [d, 2H, —CH₂—], 4.26[q, 1H, Br—CH—], 5.44 [d, 2H, O—CH—], 7.20-7.32 [m, 4H, Ar—H], 8.78 [s, 1H, Ar—H], 8.86-8.95 [m, 2H, Ar—H].

### 3-Phenoxycarbonyl-5-nitrophenylboronic acid (FL-092)

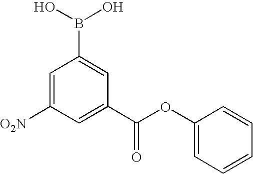

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with phenol (14 mg, 0.15 mmol) according to general procedure A, step (ii), to give 39 mg (91% yield) of the desired compound as a white needle, mp: 139-141° C. ¹H-NMR(400 MHz, 5% D₂O in d₆-DMSO): δ 7.28-7.34 [m, 3H, Ar—H], 7.44-7.51 [m, 2H, Ar—H], 8.78 [s, 1H, Ar—H], 8.86-8.92 [m, 2H, Ar—H].

### 3-(3-Nitrobenzyloxycarbonyl)-5-nitrophenylboronic acid (Compound FL-094)

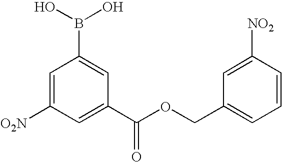

In step (i) of procedure A, oxalyl chloride (35 μl, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 3-nitrobenzyl alcohol (23 mg, 0.15 mmol) according to general procedure A, step (ii), to give 43 mg (82% yield) of the desired compound as a pale yellow powder, mp: 142-144° C. ¹H-NMR (400 MHz, 5% D₂O in d₆-DMSO): δ5.46 [s, 2H, Ar—CH_{2], 7.65}-7.70 [m, 1H, Ar—H], 7.93-7.97 [m, 1H, Ar—H], 8.09-8.12 [m, 1H, Ar—H], 8.17-8.21 [m, 1H, Ar—H], 8.62-8.94 [m, 3H, Ar—H].

### 3-(3-Chlorobenzyloxycarbonyl)-5-nitrophenylboronic acid (FL-095)

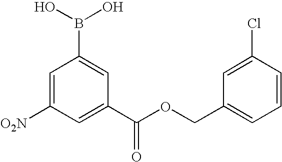

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 3-chlorobenzyl alcohol (22 mg, 0.15 mmol) according to general procedure A, step (ii), to give 44 mg (88% yield) of the desired compound as a white powder, mp: 91° C. (dec). ¹H-NMR(400 MHz, 5% D₂O in d₆-DMSO): δ5.41 [s, 2H, Ar—CH₂], 7.41-7.61 [m, 4H, Ar—H], 8.62-8.95 [m, 3H, Ar—H].

### 3-(2-Bromophenoxycarbonyl)-5-nitro-phenylboronic acid (FL-096)

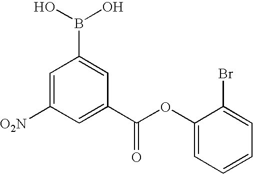

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 2-bromophenol (26 mg, 0.15 mmol) according to general procedure A, step (ii), to give 49 mg (90% yield) of the desired compound as a pale yellow powder, mp: 128-130° C. ¹H-NMR(400 MHz, 5% D₂O in d₆-DMSO): δ7.26-7.35 [m, 1H, Ar—H], 7.47-7.56 [m, 2H, Ar—H], 7.76-7.81 [m, 1H, Ar—H], 8.83-8.94 [m, 3H, Ar—H].

### 3-(1-Benzoyl-1-Phenylmethoxycarbonyl)-5-nitrophenylboronic acid (FL-097)

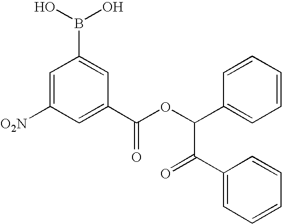

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with benzoin (32 mg, 0.15 mmol) according to general procedure A, step (ii), to give 43 mg (71% yield) of the desired compound as a white powder, mp: 237° C. (dec). ¹H-NMR(400 MHz, 5% D₂O in d₆-DMSO): δ 7.37-7.55 [m, 3H, Ar—H], 7.58-7.69 [m, 3H, Ar—H], 7.75-7.83 [m, 1H, Ar—H], 7.88-7.94 [m, 1H, Ar—H], 8.07-8.13 [m, 1H, Ar—H), 8.77 [s, 1H, Ar—H], 8.80-8.96 [m, 2H, Ar—H].

### 3-(3-Boronophenoxycarbonyl)-5-nitrophenylboronic acid (Compound FL-100)

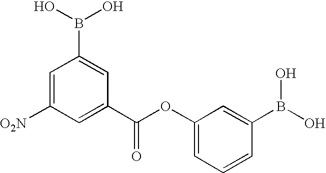

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 3-hydroxyphenylboronic acid (21 mg, 0.15 mmol) according to general procedure A, step (ii), to give 32 mg (64% yield) of the desired compound as a pale yellow powder, mp: 228-230° C. ¹H-NMR (400 MHz, 5% D₂O in d₆-DMSO): δ7.30-7.36 [m, 1H, Ar—H], 7.41-7.47 [m, 1H, Ar—H], 7.60-7.63 [m, 1H, Ar—H], 7.68-7.73 [m, 1H, Ar—H], 8.76-8.79 [m, 1H, Ar—H], 8.84-8.90 [m, 2H, Ar—H].

### 3-(3-Boronobenzyloxycarbonyl)-5-nitrophenylboronic acid (FL-101)

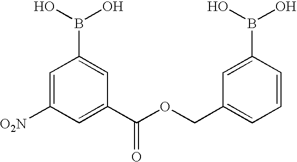

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 3-hydroxymethylphenylboronic acid (23 mg, 0.15 mmol) according to general procedure A, step (ii), to give 35 mg (68% yield) of the desired compound as a yellow semisolid. ¹H-NMR (400 MHz, 5% D₂O in d₆-DMSO): δ 5.39 [s, 2H, Ar—CH_{2], 7.27}-7.84 [m, 4H, Ar—H], 8.79-8.90 [m, 3H, Ar—H].

### 4-(2-Chloro-6-nitrophenoxycarbonyl)-3-fluorophenylboronic acid (FL-104)

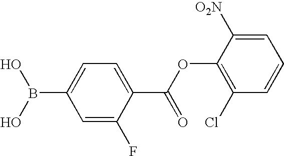

In step (i) of procedure A, oxalyl chloride (35 μL, 0.4 mmol) was added to a suspension of 3-fluoro-4-carboxylphenylboronic acid (37 mg, 0.2 mmol), 1 drop of DMF and 5 mL of anhydrous CH₂Cl₂. The reaction was stirred overnight at room temperature and then evaporated to dryness. The product obtained was reacted with 2-chloro-6-nitrophenol (26 mg, 0.15 mmol) according to general procedure A, step (ii), to give 40 mg (79% yield) of the desired compound as a pale yellow powder, mp: 174-175° C. ¹H-NMR (400 MHz, 5% D₂O in d₆-DMSO): δ7.61-7.36 [m, 1H, Ar—H], 7.71-7.74 [m, 1H, Ar—H], 7.77-7.79 [m, 1H, Ar—H], 8.07-8.11 [m, 2H, Ar—H], 8.18-8.21 [m, 2H, Ar—H].

### 3-(2,4-Dichloro-6-nitro-phenoxycarbonyl)-5-nitro-phenylboronic acid (FL-107)

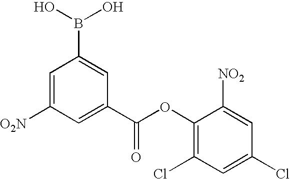

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 2,4-dichloro-6-nitrophenol (80%) (39 mg, 0.15 mmol) according to general procedure A, step (ii), to give 52 mg (87% yield) of the desired compound as a pale yellow powder, mp: 184-186° C. ¹H-NMR (400 MHz, 5% D₂O in d₆-DMSO): 8.34-8.38 [m, 1H, Ar—H], 8.82-8.84 [m, 1H, Ar—H], 8.90-8.91 [m, 1H, Ar—H], 8.94-8.96 [m, 1H, Ar—H].

### 3-(2,4-Dichloro-6-nitro-phenoxycarbonyl)-5-nitro-phenylboronic acid (FL-132)

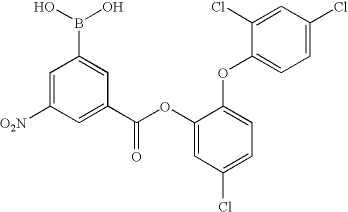

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 5-chloro-2-(2,4-dichlorophenoxy)phenol (43 mg, 0.15 mmol) according to general procedure A, step (ii), to give 51 mg (71% yield) of the desired compound as a pale yellow powder, mp: 193° C. ¹H-NMR (400 MHz, 5% D₂O in d₆-DMSO): δ6.6₁₋₆.94 [m, 3H, Ar—H], 7.15-8.07 [m, 3H, Ar—H], 8.71-8.76 [m, 1H, Ar—H], 8.84-8.89 [m, 1H, Ar—H], 8.89-8.96 [m, 1H, Ar—H].

### 3-(4-Coumarinoxycarbonyl)-5-nitrophenylboronic acid (FL-133)

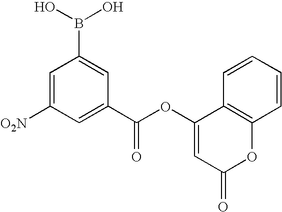

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 4-hydroxycoumarin (24 mg, 0.15 mmol) according to general procedure A, step (ii), to give 36 mg (67% yield) of the desired compound as a white powder, mp: 230-232° C. ¹H-NMR (400 MHz, 5% D₂O in d₆-DMSO): δ7.12-7.22 [m, 1H, Ar—H], 7.23-7.36 [m, 2H, Ar—H], 7.55-7.64 [m, 1H, Ar—H], 7.72-7.86 [m, 1H, Ar—H], 8.60 [s, 1H, Ar—H], 8.72 [s, 1H, Ar—H], 8.77 [s, 1H, Ar—H].

### 3-(2,4,6-Trichlorophenoxycarbonyl)-5-nitro-phenylboronic acid (FL-139)

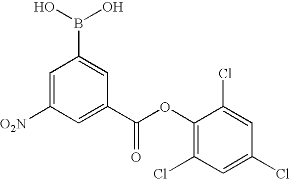

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 2,4,6-trichlorophenol (30 mg, 0.15 mmol) according to general procedure A, step (ii), to give 45 mg (77% yield) of the desired compound as a white powder, mp: 217° C. (dec). ¹H-NMR (400 MHz, 5% D₂O in d₆-DMSO): δ7.24 [s, 2H, Ar—H], 8.77-8.80 [m, 1H, Ar—H], 8.90-8.96 [m, 2H, Ar—H].

### 3-(1-Naphthalenemethoxycarbonyl)-5-nitrophenylboronic acid (FL-148)

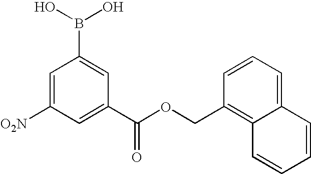

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 1-naphthalenemethanol (24 mg, 0.15 mmol) according to general procedure A, step (ii), to give 38 mg (73% yield) of the desired compound as a pale yellow powder, mp: 230° C. (dec). ¹H-NMR (400 MHz, 5% D₂O in d₆-DMSO): δ 5.84 [s, 2H, Ar—CH₂—], 7.47-7.61 [m, 3H, Ar—H], 7.65-7.72 [m, 1H, Ar—H], 7.92-7.99 [m, 1H, Ar—H], 8.02-8.16 [m, 1H, Ar—H], 8.58 [s, 1H, Ar—H], 8.68 [s, 1H, Ar—H], 8.77 [s, 1H, Ar—H].

### 3-(1,1-Diphenylmethoxycarbonyl)-5-nitrophenylboronic acid (FL-149)

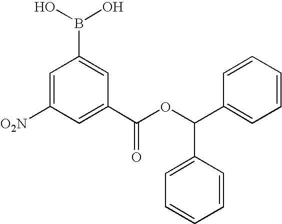

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with (40 mg, 0.15 mmol) according to general procedure A, step (ii), to give 40 mg (70% yield) of the desired compound as a pale yellow powder, mp: 217° C. (dec). ¹H-NMR (400 MHz, 5% D₂O in d₆-DMSO): δ 5.66 [s, 2H, Ar—CH—], 7.13-7.18 μm, 1H, Ar—CH—], 7.23-7.31 [m, 3H, Ar—H], 7.31-7.46 [m, 5H, Ar—H], 7.48-7.54 [m, 1H, Ar—H], 8.73 [s, 1H, Ar—H], 8.83-8.87 [m, 2H, Ar—H].

### 3-(2-Naphthoxycarbonyl)-5-nitrophenylboronic acid (FL-151)

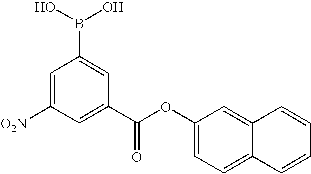

In step (i) of procedure A, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 2-naphthol (22 mg, 0.15 mmol) according to general procedure A, step (ii), to give 41 mg (82% yield) of the desired compound as a pale yellow powder, mp: 256° C. (dec). ¹H-NMR (400 MHz, 5% D₂O in d₆-DMSO): δ7.42-7.54 [m, 3H, Ar—H], 7.78-8.02 [m, 4H, Ar—H], 8.74-8.79 [m, 1H, Ar—H], 8.80-8.86 [m, 1H, Ar—H], 8.86-8.91 [m, 1H, Ar—H].

### 3-(3-Boronobenzyloxycarbonyl)-5-nitrophenylboronic acid (Compound FL-171)

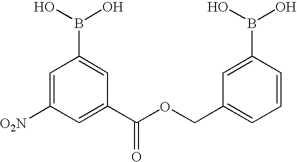

In step (i) of procedure A, oxalyl chloride (35 μl, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 3-hydroxymethylphenylboronic acid (23 mg, 0.15 mmol) according to general procedure A, step (ii), to give 35 mg (68% yield) of the desired compound as a yellow semisolid. ¹H-NMR (400 MHz, 5% D₂O in d₆-DMSO): δ 5.39 [s, 2H, Ar—CH₂], 7.27-7.84 [m, 4H, Ar—H], 8.79-8.90 [m, 3H, Ar—H].

1.2 General Procedure B: Synthesis of Boronobenzoic Acid Phenylene Esters

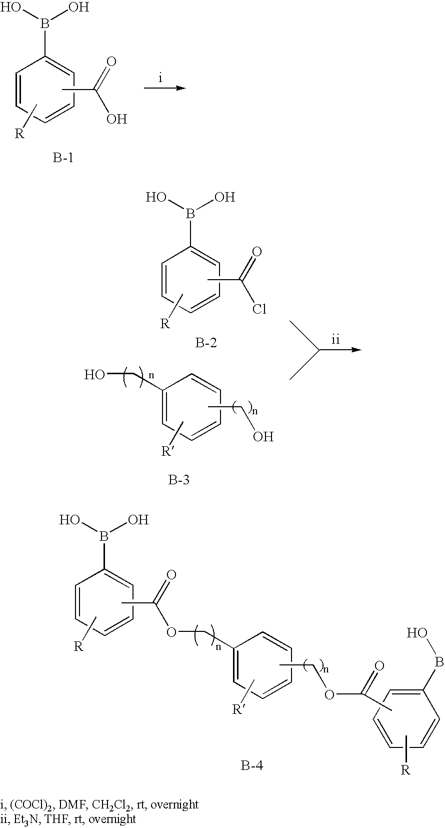

In step (i), oxalyl chloride (35 μL, 0.4 mmol) was added to a suspension of compound B-1 (0.2 mmol), 1 drop of DMF and 5 mL of dried CH₂Cl₂. The reaction mixture was stirred at room temperature overnight and then evaporated to dryness to afford acid chloride B-2 as a solid, which was used without further purification in step (ii).

In step (ii), a suspension of the acid chloride B-2 (0.2 mmol, obtained from step (i) above) in 5 mL of dried THF was added dropwise to an ice-cold solution of B-3 (0.075 mmol), anhydrous triethyl amine (42 μL, 0.3 mmol) and 10 mL of anhydrous THF. The reaction mixture was stirred at room temperature overnight and then evaporated to dryness. It was dissolved in 25 mL of ethyl acetate and washed with 1N aqueous HCl, 10% aqueous NaHCO₃, saturated brine solution, dried (Na₂SO₄) and then concentrated. The residue was purified by flash chromatography eluting with MeOH-ethyl acetate (1:10). The product yielded from step (ii) is B-4.

### 3-Borono-5-nitrobenzoic acid 1,4-phenylene ester (FL-078)

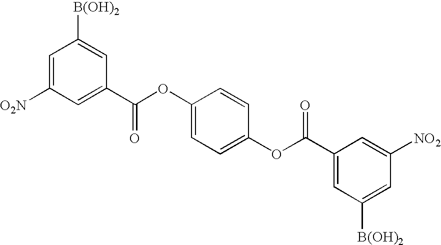

In step (i) of procedure B, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with hydroquinone (9 mg, 0.075 mmol) according to general procedure B, step (ii), to give 32 mg (86% yield) of the desired compound as a white powder, mp: 271-273° C. ¹H-NMR(300 MHz, 5% D₂O in d₆-DMSO): δ 7.43 [s, 4H, Ar—H], 8.79 [s, 2H, Ar—H], 8.84 [s, 2H, Ar—H], 8.86 [s, 2H, Ar—H].

### 3-Borono-5-nitrobenzoic acid 2-chloro-1,4-phenylene ester (FL-079)

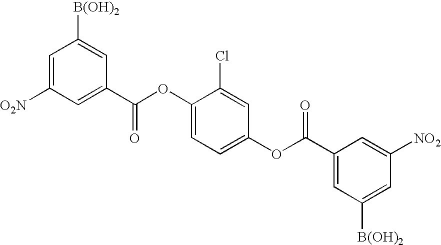

In step (i) of procedure B, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with chlorohydroquinone (11 mg, 0.075 mmol) according to general procedure B, step (ii), to give 31 mg (78% yield) of the desired compound as a white powder, mp: 264-266° C. ¹H-NMR(300 MHz, 5% D₂O in d₆-DMSO): δ7.46-7.79 [m, 3H, Ar—H], 8.79 [d, 1H, Ar—H], 8.88-8.92 [m, 2H, Ar—H].

### 4-Borono-2-fluorobenzoic acid 2-chloro-1,4-phenylene ester (FL-103)

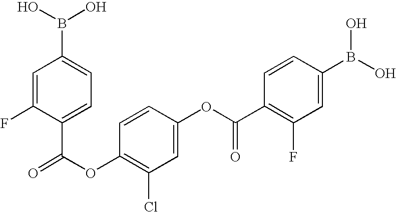

In step (i) of procedure B, oxalyl chloride (35 μL, 0.4 mmol) was added to a suspension of 3-fluoro-4-carboxylphenylboronic acid (37 mg, 0.2 mmol), 1 drop of DMF and 5 mL of anhydrous CH₂Cl₂. The reaction was stirred overnight at room temperature and then evaporated to dryness. The product obtained was reacted with and chlorohydroquinone (11 mg, 0.075 mmol) according to general procedure B, step (ii), to give 26 mg (74% yield) of the desired compound as a pale yellow powder, mp: 228-230° C. ¹H-NMR (400 MHz, 5% D₂O in d₆-DMSO): δ6.52-6.55 [m, 1H, Ar—H], 6.68-6.76 [m, 2H, Ar—H], 6.94-7.06 [m, 1H, Ar—H], 7.21-7.34 [m, 1H, Ar—H], 7.57-7.78 [m, 3H, Ar—H], 7.99-8.11 [m, 1H, Ar—H].

### 3-Borono-5-nitrobenzoic acid 1,3-phenylene ester (FL-106)

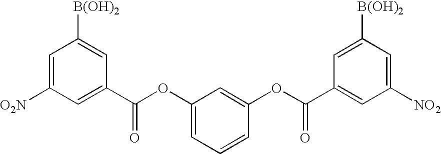

In step (i) of procedure B, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with resorcinol (9 mg, 0.075 mmol) according to general procedure B, step (ii), to give 26 mg (70% yield) of the desired compound as a pale yellow powder, mp: 134° C. (dec). ¹H-NMR (400 MHz, 5% D₂O in d₆-DMSO): δ 6.14-6.18 [m, 2H, Ar—H], 7.33-7.45 [m, 2H, Ar—H], 8.75-8.82 [m, 2H, Ar—H], 8.85-8.90 [m, 4H, Ar—H].

### 3-Borono-5-nitrobenzoic acid 1,2-benzenedimethanol ester (FL-136)

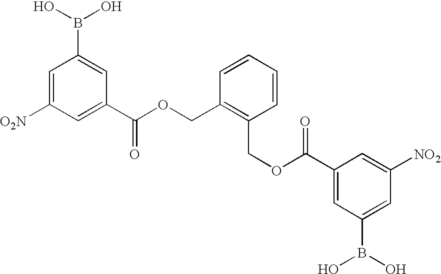

In step (i) of procedure B, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 1,2-benzenedimethanol (10 mg, 0.075 mmol) according to general procedure B, step (ii), to give 21 mg (53% yield) of the desired compound as a pale yellow powder, mp: 142° C. (dec). ¹H-NMR (400 MHz, 5% D₂O in d₆-DMSO): δ 5.64 [s, 4H, CH_{2], 6.14}-6.18 [m, 2H, Ar—H], 7.33-7.45 [m, 4H, Ar—H], 8.77-8.84 [m, 2H, Ar—H], 8.87-8.93 [m, 4H, Ar—H].

### 3-Borono-5-nitrobenzoic acid 2,3,5,6-tetrachloro-1,4-phenylene ester (FL-141)

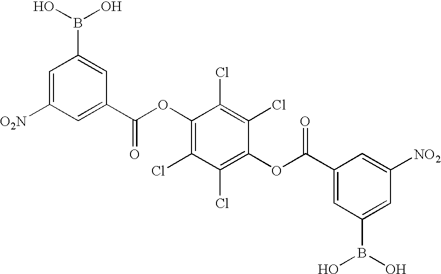

In step (i) of procedure B, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted tetrachlorohydroquinone (19 mg, 0.075 mmol) according to general procedure B, step (ii), to give 33 mg (69% yield) of the desired compound as a white powder, mp: 252° C. ¹H-NMR(300 MHz, 5% D₂O in d₆-DMSO): 8.71-8.75 [m, 2H, Ar—H], 8.93-8.99 [m, 4H, Ar—H].

### 3-Borono-5-nitrobenzoic acid, 1,4-dihydroxynaphthalene ester (FL-167)

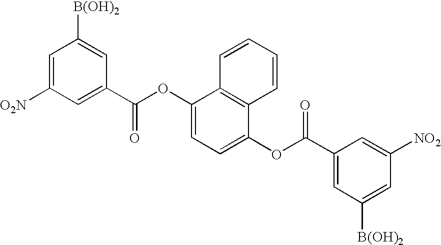

In step (i) of procedure B, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with 1,4-naphthalenediol (12 mg, 0.075 mmol) according to general procedure B, step (ii), to give 24 mg (59% yield) of the desired compound as a pale yellow powder, mp: 270° C. (dec). ¹H-NMR(300 MHz, 5% D₂O in d₆-DMSO): δ 6.89 [s, 2H, Ar—H], 7.68-7.74 [m, 2H, Ar—H], 8.08-8.13 [m, 2H, Ar—H], 8.70 [s, 2H, Ar—H], 8.85 [s, 2H, Ar—H], 8.93 [s, 2H, Ar—H].

### 3-Borono-5-nitrobenzoic acid, 2-tert-butyl-1,4-phenylene ester (FL-169)

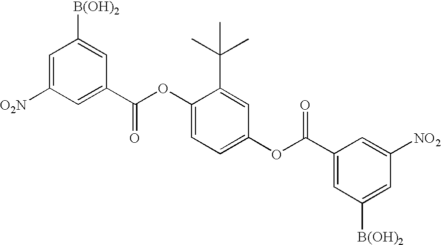

In step (i) of procedure B, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with tert-butylhydroquinone (12 mg, 0.075 mmol) according to general procedure B, step (ii), to give 22 mg (43% yield) of the desired compound as a brown powder, mp: 268° C. (dec). ¹H-NMR(300 MHz, 5% D₂O in d₆-DMSO): δ 1.37 [s, 9H, —CH_{3], 7.10}-7.13 [m, 1H, Ar—H], 7.27-7.31 [m, 1H, Ar—H], 7.51-7.54 [m, 1H, Ar—H], 8.65 [s, 2H, Ar—H], 8.77 [s, 2H, Ar—H], 8.88 [s, 2H, Ar—H].

1.3 General Procedure C: Synthesis of Acyloxyphenylboronic Acids

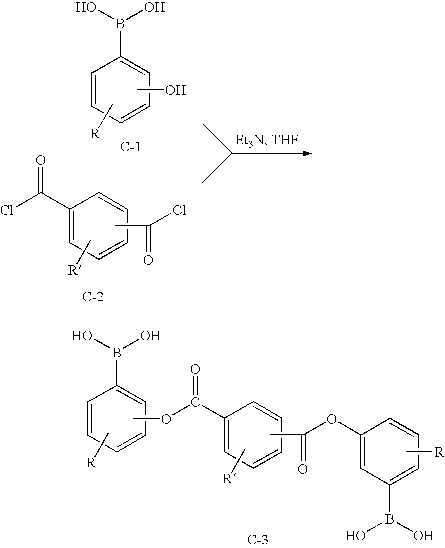

To an ice-cold solution of C-1 (0.15 mmol), dried triethyl amine (28 μL, 0.2 mmol) and 10 mL of dried THF was added dropwise a solution of C-2 (0.1 mmol) in 5 mL of dried THF. The reaction mixture was stirred at room temperature overnight and then evaporated to dryness. It was dissolved in 25 mL of ethyl acetate and washed with 1N aqueous HCl, 10% aqueous NaHCO₃, saturated brine solution, dried (Na₂SO₄) and concentrated. The residue was purified by flash chromatography eluting with MeOH-ethyl acetate (1:10). The product yielded is C-3.

### 1,4-Benzenedicarboxylic acid, di 3-boronophenyl ester (Compound FL-156)

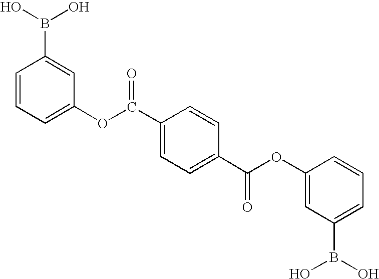

3-Hydroxyphenylboronic acid (21 mg, 0.15 mmol) and terephthaloyl chloride (21 mg, 0.1 mmol) were reacted according to general procedure C, to give 16 mg (51% yield) of the desired compound as a white powder, mp: 214° C. (dec). ¹H-NMR (400 MHz, 5% D₂O in d₆-DMSO): δ7.28-7.34 [m, 2H, Ar—H], 7.38-7.46 [m, 2H, Ar—H], 7.59-7.63 [m, 2H, Ar—H], 7.68-7.73 [m, 2H, Ar—H], 8.30 [s, 4H, Ar—H].

1.4 General Procedure D: Synthesis of Amide of Boronoanilines

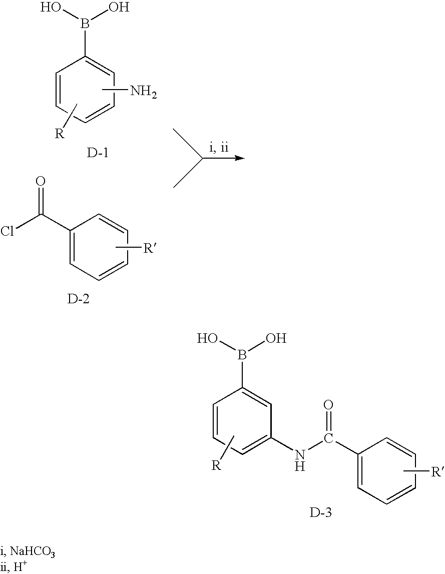

To an ice-cold solution of D-1 (0.5 mmol) and NaHCO₃ (105 mg, 1.25 mmol) in 10 mL of water and 10 mL of ether was add dropwise D-2 (0.5 mmol) over a period of 30 min. The reaction mixture was kept at 0° C. for 1 h and then stirred at room temperature overnight. It was then extracted twice with 10 mL of ethyl ether. The aqueous solution was acidified with 1N aqueous HCl and extracted twice with 10 mL of ethyl acetate. The combined organic layers were washed with water and saturated brine solution, dried (Na₂SO₄) and concentrated. The residue was recrystallized from ethyl acetate/hexane. The product yielded is D-3.

### 3-(2-Fluorobenzamido)-5-carboxylphenylboronic acid (Compound FL-083)

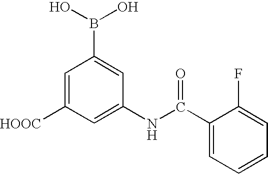

2-Fluorobenzoyl chloride (60 μL, 0.5 mmol) and 3-amino-5-carboxylphenylboronic acid (91 mg, 0.5 mmol) were reacted according to general procedure D, to give 94 mg (62% yield) of the desired compound as a white powder, mp: 224-225° C. ¹H-NMR(300 MHz, 5% D₂O in d₆-DMSO): δ 7.21-7.38 [m, 2H, Ar—H], 7.48-7.72 [m, 2H, Ar—H], 8.12-8.33 [m, 3H, Ar—H].

### 3-(2-Fluorobenzamido)-5-nitrophenylboronic acid (FL-084)

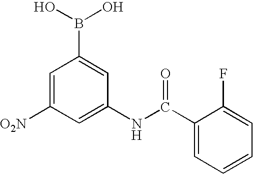

2-Fluorobenzoyl chloride (60 μL, 0.5 mmol) and 3-amino-5-nitrophenylboronic acid hydrochloride (109 mg, 0.5 mmol) were reacted according to general procedure D, to give 82 mg (54% yield) of the desired compound as a white powder, mp: 212-214° C. ¹H-NMR(300 MHz, 5% D₂O in d₆-DMSO): δ7.22-7.42 [m, 2H, Ar—H], 7.53-7.63 [m, 1H, Ar—H], 7.64-7.72 [m, 1H, Ar—H], 8.34 [s, 1H, Ar—H], 8.37 [s, 1H, Ar—H], 8.79 [s, 1H, Ar—H].

### 3-(2,6-Dichlorobenzamido)-5-nitrophenylboronic acid (FL-108)

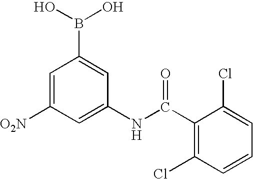

2,6-Dichlorobenzoyl chloride (72 μL, 0.5 mmol) and 3-amino-5-carboxylphenylboronic acid hydrochloride (109 mg, 0.5 mmol) were reacted according to general procedure D, to give 113 mg (64% yield) of the desired compound as a white powder, mp: 267° C. (dec). ¹H-NMR(300 MHz, 5% D₂O in d₆-DMSO): δ7.46-7.53 [m, 1H, Ar—H], 7.54-7.60 [m, 2H, Ar—H], 8.27 [s, 1H, Ar—H], 8.40 [s, 1H, Ar—H], 8.74 [s, 1H, Ar—H].

### 3-(2,6-Dichlorobenzamido)-4-chlorophenylboronic acid (FL-109)

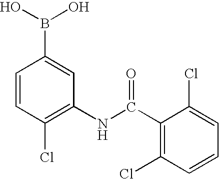

2,6-Dichlorobenzoyl chloride (72 μL, 0.5 mmol) and 3-amino-4-chlorophenylboronic acid hydrochloride (104 mg, 0.5 mmol) were reacted according to general procedure D, to give 98 mg (57% yield) of the desired compound as a pale yellow powder, mp: 246° C. (dec). ¹H-NMR(300 MHz, 5% D₂O in d₆-DMSO): δ7.41-7.57 [m, 4H, Ar—H], 7.62-7.68 [m, 1H, Ar—H], 7.95-8.01 [m, 1H, Ar—H].

### 3-(2,6-Dichlorobenzamido)-4-methylphenylboronic acid (FL-110)

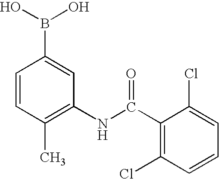

2,6-Dichlorobenzoyl chloride (72 μL, 0.5 mmol) and 3-amino-4-methylphenylboronic acid hydrochloride (94 mg, 0.5 mmol) were reacted according to general procedure D, to give 79 mg (49% yield) of the desired compound as a white powder, mp: 234-236° C. ¹H-NMR(300 MHz, 5% D₂O in d₆-DMSO): δ 2.26 [s,3H,—CH₃], 7.15-7.25 [m, 1H, Ar—H], 7.40-7.47 [m, 1H, Ar—H], 7.47-7.60 [m, 3H, Ar—H], 7.71-7.77 [m, 1H, Ar—H].

### 3-Benzamido-5-nitrophenylboronic acid (FL-113)

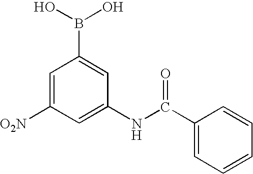

Benzoyl chloride (58 μL, 0.5 mmol) and 3-amino-5-nitrophenylboronic acid hydrochloride (109 mg, 0.5 mmol) were reacted according to general procedure D, to give 94 mg (66% yield) of the desired compound as a pale yellow powder, mp: 227-229° C. ¹H-NMR(300 MHz, 5% D₂O in d₆-DMSO): δ7.49-7.54 [m, 2H, Ar—H], 7.56-7.61 [m, 1H, Ar—H], 7.94-7.99 [m, 2H, Ar—H], 8.36 [s, 1H, Ar—H], 8.46 [s, 1H, Ar—H], 8.84 [s, 1H, Ar—H].

1.5 General Procedure E: Synthesis of Boronobenzoic Acid Phenylene Amides

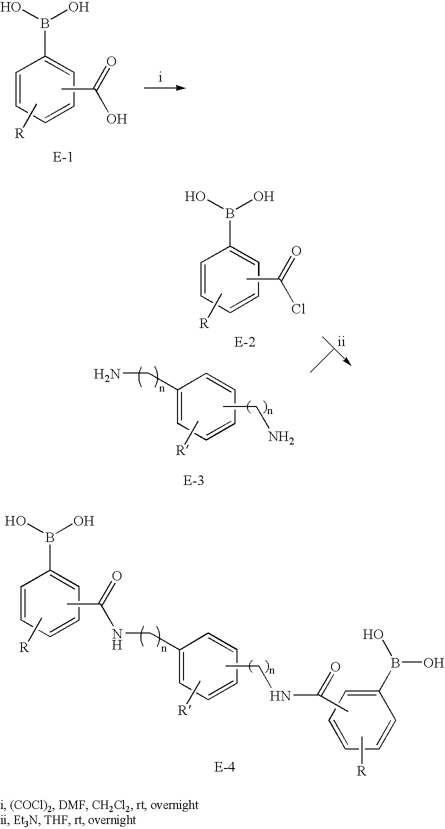

In step (i), oxalyl chloride (35 μL, 0.4 mmol) was added to a suspension of compound E-1 (0.2 mmol), 1 drop of DMF and 5 mL of dried CH₂Cl₂. The reaction mixture was stirred at room temperature overnight and then evaporated to dryness to afford acid chloride E-2 as a solid, which was used without further purification in step (ii).

In step (ii), a suspension of the acid chloride E-2 (0.2 mmol, obtained from step (i) above) in 5 mL of dried THF was added dropwise to an ice-cold solution of E-3 (0.075 mmol), anhydrous triethylamine (42 μL, 0.3 mmol) and 10 mL of anhydrous THF. The reaction mixture was stirred at room temperature overnight and then evaporated to dryness. It was dissolved in 25 mL of ether and washed with 1N aqueous HCl, 10% aqueous NaHCO₃, saturated brine solution, dried (Na₂SO₄) and then concentrated. The residue was recrystallized from ethyl acetate/hexane. The product yielded from step (ii) is E-4.

### 3-Borono-5-nitrobenzoyl-1,4-phenylenediamine (FL-166)

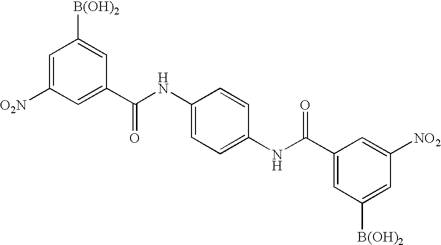

In step (i) of procedure E, oxalyl chloride (35 μL, 0.4 mmol) was added to a suspension of (3-carboxyl-5-nitrophenyl)boronic acid (42 mg, 0.2 mmol), 1 drop of DMF and 5 mL of anhydrous CH₂Cl₂. The reaction was stirred overnight at room temperature and then evaporated to dryness. The product obtained was reacted with p-phenylenediamine (8 mg, 0.075 mmol) according to general procedure E, step (ii), to give 29 mg (78% yield) of the desired compound as pale yellow powder, mp: 254-256° C. ¹H-NMR(300 MHz, 5% D₂O in d₆-DMSO): δ7.73 [s, 4H, Ar—H], 8.67-8.70 [m, 2H, Ar—H], 8.72-8.78 [m, 4H, Ar—H].

### 1.6 Additional Synthetic Examples

### 3-amino-5-carboxylphenylboronic acid (FL-088)

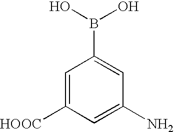

To make desired compound, a solution of 3-carboxyl-5-nitrophenylboronic acid (422 mg, 2 mmol) in absolute ethanol (5 ml) was hydrogenated in the presence of Raney Nickel (150 mg) for 6 hours. The catalyst was removed by filtration and the solvent was evaporated to dryness, then the residue was recrystallized from water to give 257 mg (71% yield) of the desired compound as a pale yellow powder, mp: 210-212° C. (Ref. mp: 212-214° C.). ¹H-NMR(400 MHz, 5% D₂O in d₆-DMSO): δ7.06 [s, 1H, Ar—H], 7.16 [s, 1H, Ar—H], 7.55 [s, 1H, Ar—H] (Torssell, K.; Meyer, H.; Zacharias, B. Arkiv Kemi 1957, 10, 497-505).

### 3-(2,6-dichlorophenylcarbonyloxy)phenyl boronic acid (FL-099)

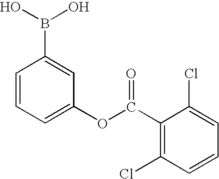

To make desired compound, a solution of 2,6-dichlorobenzoyl chloride (29 μL, 0.2 mmol) in 5 mL of dried THF was added dropwise to an ice-cold solution of 3-hydroxyphenylboronic acid (21 mg, 0.15 mmol), dried triethylamine (42 μL, 0.3 mmol) and 10 mL of dried THF. The reaction mixture was stirred at room temperature overnight and then evaporated to dryness. It was dissolved in 25 mL of ethyl acetate and washed with 1N aqueous HCl, 10% aqueous NaHCO₃, saturated brine solution, dried (Na₂SO₄) and then concentrated. The residue was purified by flash chromatography eluting with MeOH-ethyl acetate (1:10) to give 38 mg (81% yield) of desired compound as a white powder, mp: 238° C. (dec). ¹H-NMR (400 MHz, 5% D₂O in d₆-DMSO): δ7.08-7.19 [m, 1H, Ar—H], 7.22-7.30 [m, 1H, Ar—H], 7.22-7.30 [m, 1H, Ar—H], 7.42-7.50 [m, 1H, Ar—H], 7.54-7.60 [m, 1H, Ar—H], 7.60-7.68 [m, 2H, Ar—H], 7.73-7.78 [m, 3H, Ar—H].

### 3-benzenesulfonamido-5-nitrophenylboronic acid (FL-114)

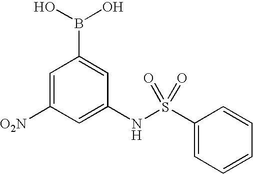

To make desired compound, a solution of benzenesulfonyl chloride (19 μL, 0.15 mmol) in 5 mL of dried THF was added dropwise to an ice-cold solution of 3-amino-5-nitrophenylboronic acid, HCl (33 mg, 0.15 mmol), dried triethylamine (63 μL, 0.45 mmol) and 10 mL of dried THF. The reaction mixture was stirred at room temperature overnight and then evaporated to dryness. It was dissolved in 25 mL of ether and washed with 1N aqueous HCl, 10% aqueous NaHCO₃, saturated brine solution, dried (Na₂SO₄) and then concentrated. The residue was purified by flash chromatography eluting with MeOH-ethyl acetate (1:10) to give 37 mg (77% yield) of desired compound as a white powder, mp: 238° C. (dec). ¹H-NMR (400 MHz, 5% D₂O in d₆-DMSO): δ7.49-7.61 [m, 3H, Ar—H], 7.72-7.78 [m, 2H, Ar—H], 7.82-7.85 [m, 1H, Ar—H], 7.93-7.96 [m, 1H, Ar—H], 8.25-8.29 [m, 1H, Ar—H].

### Tris-(3-borono-5-nitrobenzoic acid), 1,3,5-trihydroxybenzene ester (FL-164)

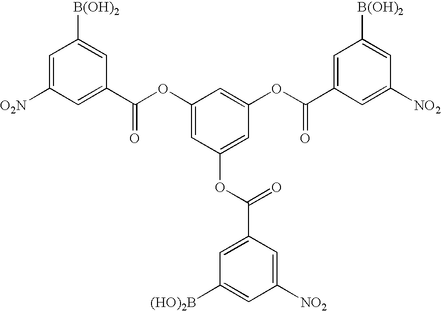

In step (i), oxalyl chloride (79 μL, 0.9 mmol) was added to a suspension of (3-carboxyl-5-nitrophenyl)boronic acid (95 mg, 0.45 mmol), 1 drop of DMF and 10 mL of dried CH₂Cl₂. The reaction mixture was stirred at room temperature overnight and then evaporated to dryness to afford acid chloride as a yellow solid, which was used without further purification in step (ii).

In step (ii), a suspension of the above acid chloride (0.45 mmol, obtained from step (ii)) in 5 mL of dried THF was added dropwise to an ice-cold solution of phloroglucinol (13 mg, 0.1 mmol), anhydrous triethylamine (63 μL, 0.45 mmol) and 10 mL of anhydrous THF. The reaction mixture was stirred at room temperature overnight and then evaporated to dryness. It was dissolved in 25 mL of ethyl acetate and washed with 1N aqueous HCl, 10% aqueous NaHCO₃, saturated brine solution, dried (Na₂SO₄) and then concentrated. The residue was purified by flash chromatography eluting with MeOH-ethyl acetate (1:10) to give 17 mg (24% yield) of the desired compound as a pale yellow powder, mp: 274° C. (dec). ¹H-NMR (400 MHz, 5% D₂O in d₆-DMSO): δ7.46 [s, 1H, Ar—H], 8.72-8.76 [m, 1H, Ar—H], 8.78-8.86 [m, 2H, Ar—H].

### Tris(3-borono-5-nitrobenzoic acid), triethanolamine ester (FL-165)

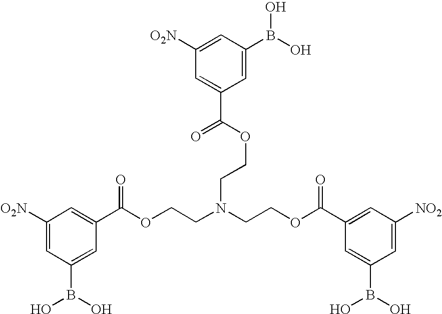

In step (i), oxalyl chloride (79 μL, 0.9 mmol) was added to a suspension of (3-carboxyl-5-nitrophenyl)boronic acid (95 mg, 0.45 mmol), 1 drop of DMF and 10 mL of dried CH₂Cl₂. The reaction mixture was stirred at room temperature overnight and then evaporated to dryness to afford acid chloride as a yellow solid, which was used without further purification in step (ii).

In step (ii), a suspension of the above acid chloride (0.45 mmol, obtained from step (ii)) in 5 mL of dried THF was added dropwise to an ice-cold solution of triethanolamine (15 mg, 0.1 mmol), anhydrous triethylamine (42 μL, 0.3 mmol) and 10 mL of anhydrous THF. The reaction mixture was stirred at room temperature overnight and then evaporated to dryness. It was dissolved in 25 mL of ethyl acetate and washed with water, 10% aqueous NaHCO₃, saturated brine solution, dried (Na₂SO₄) and then concentrated. The residue was purified by flash chromatography eluting with MeOH-ethyl acetate (1:10) to give 14 mg (19% yield) of the desired compound as a pale yellow powder, mp: 235° C. (dec). ¹H-NMR (400 MHz, 5% D₂O in d₆-DMSO): δ2.05 [t, 3H, —CH₂—], 4.05 [t, 2H, —CH₂—], 8.59-8.63 [m, 3H, Ar—H], 8.71-8.76 [m, 3H, Ar—H], 8.77-8.81 [m, 3H, Ar—H].

### 3-borono-5-nitrobenzoyl-L-phenylalanine ethyl ester (FL-170)

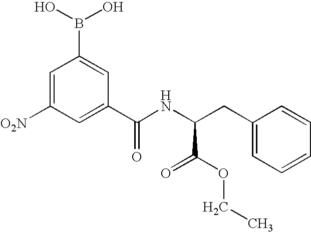

In step (i), oxalyl chloride (35 μL, 0.4 mmol) was added to a suspension of (3-carboxyl-5-nitrophenyl)boronic acid (42 mg, 0.2 mmol), 1 drop of DMF and 10 mL of dried CH₂Cl₂ The reaction mixture was stirred at room temperature overnight and then evaporated to dryness to afford acid chloride as a yellow solid, which was used without further purification in step (ii).

In step (ii), a solution of the above acid chloride (0.2 mmol, obtained from step (ii)) in 5 mL of dried THF was added dropwise to an ice-cold solution of L-phenylalanine ethyl ester hydrochloride (34 mg, 0.15 mmol), anhydrous triethylamine (63 μL, 0.45 mmol) and 10 mL of anhydrous THF. The reaction mixture was stirred at room temperature overnight and then evaporated to dryness. It was dissolved in 25 mL of ethyl acetate and washed with 1N aqueous HCl, 10% aqueous NaHCO₃, saturated brine solution, dried (Na₂SO₄) and then concentrated. The residue was purified by flash chromatography eluting with MeOH-ethyl acetate (1:10) to give 31 mg (54% yield) of the desired compound as a pale yellow powder, mp: 264° C. (dec). ¹H-NMR (400 MHz, 5% D₂O in d₆-DMSO): δ1.35 [t, 2H, —CH₂CH₃], 3.13 [dd, 1H, —CH₂—], 4.02 [q, 2H, —CH₂CH₃], 4.71 [m, 1H, —CH—], 7.05-7.37 [m, 5H, Ar—H], 8.57-8.63 [m, 1H, Ar—H], 8.67-8.72 [m, 2H, Ar—H], 8.73-8.80 [m, 1H, Ar—H].

### 3-(3-boronobenzyloxycarbonyl)-5-aminophenylboronic acid (FL-171)

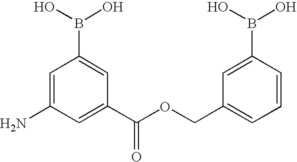

To make the desired compound, a solution of 3-(3-boronobenzyloxycarbonyl)-5-nitrophenylboronic acid (172 mg, 0.5 mmol) in absolute ethanol (10 ml) was hydrogenated in the presence of Raney Nickel (80 mg) for 4 hours. The catalyst was removed by filtration and the solvent was evaporated to dryness, then the residue was recrystallized from ethanol/H₂O to give 60 mg (38% yield) of the desired compound as a pale yellow semisolid. ¹H-NMR(300 MHz, 5% D₂O in d₆-DMSO): δ5.25 [s, 2H, Ar—CH₂], 6.56-6.97 [m, 2H, Ar—H], 7.15-8.09 [m, 5H, Ar—H].

### (3-carboxy-5-hydroxyphenyl)boronic acid (FL-172)

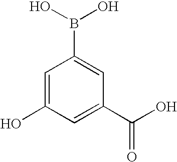

To make the desired compound, 3-amino-5-carboxylphenylboronic acid hydrochloride (22 mg, 0.1 mmol) was suspended in 2 mL of 50% H₂SO₄ and treated at −5° C. with a solution of NaNO₂ (8 mg, 0.1 mmol) in 1 mL of water. After the mixture had been stirred for 1 h at this temperature, water (10 mL) was added and the mixture was warmed to 60° C. until the evolution of gas ceased. The dark brown solution was extracted twice with ether, and the extracts were washed with water and brine and dried with Na₂SO₄. The solvent was evaporated to dryness, then the residue was recrystallized from methanol to give 5 mg (27% yield) of the desired compound as a pale yellow powder, mp: 229° C. (dec). ¹H-NMR(300 MHz, 5% D₂O in d₆-DMSO): δ7.19-7.41 [m, 2H, Ar—H], 7.77-7.87 [m, 1H, Ar—H].

### 3-Benzyloxycarbonyl-5-nitrophenylboronic acid (Compound FL-1201)

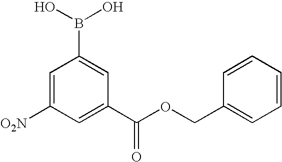

FL-1201 is available from Combi-Blocks, Inc. (San Diego, Calif., Cat. No. BB-2188).

### 3-(Benzylcarbamoyl)phenylboronic acid (Compound FL-1010)

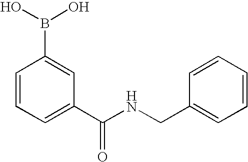

FL-1010 is available from Combi-Blocks, Inc. (San Diego, Calif., Cat. No. BB-3055).

### Example 2

### Cloning of Recombinant SARS-Associated 3CL^{pro} Protease

cDNA corresponding to the SARS 3CL protease gene (Tor2 strain, GenBank #AY274119) inserted in a pBR194c vector was kindly provided by British Columbia Cancer Agency Branch (Vancouver, British Columbia, Canada). Competent cells (XL-1 Blue, Stratagene) were transformed for plasmid propagation under ampicillin selection. DNA plasmid was isolated (Plasmid Midi Kit, Qiagen) and the gene was amplified by PCR with appropriate primers using Pfu Turbo DNA Polymerase (Stratagene). To prevent artifacts afterward, the original plasmid was degraded by DpnI (Stratagene) digestion reaction (1 hour at 37° C.) followed by inactivation of DpnI (20 minutes at 80° C.). Cloning reaction was performed by blunt-end directional cloning (Champion pET Directional TOPO Expression and Cloning Kit, Invitrogen) by topo-isomerase reaction in a pET100 vector, where the protein expression is chemically induced and under the control of the T7 promoter. The protease gene was cloned in frame with an N-terminal peptide containing a poly-histidine tag for further purification by affinity chromatography. An enterokinase recognition site was present to remove the amino terminal tag after purification. One Shot TOP10 competent cells (Invitrogen) were transformed with the product from the cloning reaction. DNA plasmid was purified and the gene insertion and its directionality, as well as the integrity of pET100 vector were confirmed by DNA sequencing. BL21 Star DE3 (Invitrogen) competent cells were transformed for protein expression under ampicillin selection and IPTG induction.

### Example 3

### Expression and Purification of Recombinant SARS-Associated 3CL^{pro} Protease

Plasmid-encoded SARS 3CL protease was expressed as a soluble fraction in BL21 Star DE3 Escherichia coli competent cells (Invitrogen). Cells were grown in LB supplemented with ampicillin (50 μg/ml) at 37° C., induced with IPTG when the optical density was ˜0.8 and harvested after 3-4 hours. Cells were re-suspended in lysis buffer (potassium phosphate 50 mM, pH 7.8, sodium chloride 400 mM, potassium chloride 100 mM, glycerol 10%, triton-X 0.5%, imidazole 10 mM) and broken with three passes through a French pressure cell (>16,000 psi). Cell debris was collected by centrifugation (20,000g at 4° C. for 20 min.). The supernatant was diluted 1:3 with binding buffer (sodium phosphate 50 mM, pH 7.5, sodium chloride 0.3 M, imidazole 10 mM), filtered using a 0.22 μm pore size filter (Millipore) and applied directly to a nickel-affinity column (H is Select, Sigma) which had been pre-equilibrated with five column volumes of the binding buffer. The protease was eluted with a linear gradient of elution buffer (sodium phosphate 50 mM, pH 7.5, sodium chloride 0.3 M, imidazole 250 mM) at fractions corresponding to 0-30% elution buffer. Protease fractions were pooled and concentrated. During concentration the elution buffer was exchanged gradually for storage buffer (sodium phosphate 10 mM, pH 7.4, sodium chloride 10 mM, DTT 1 mM, EDTA 0.5 mM). The poly-histidine tag of the fusion protein was cut through incubation with 0.1 units of enterokinase (Invitrogen) for 48 hours at 4° C. Efficiency of the cleavage reaction was inspected by SDS PAGE. This reaction mixture was passed again through the nickel-affinity column and the flow-through containing the protease was collected. The sample was diluted 4-fold with storage buffer and then concentrated (>10 mg/ml). The purified protein was then stored at −20° C. Purity of the sample was higher than 95%, assessed by SDS PAGE.

### Example 4

### Enzymatic Characterization of Recombinant SARS-Associated 3CL^{pro} Protease

The activity of the SARS protease was determined by continuous kinetic assays using the fluorogenic substrate Dabcyl-Leu-Ala-Gln-Ala-Val-Arg-Ser-Ser-Ser-Arg-Edans (Bachem). The hydrolysis of the substrate is accompanied by a proportional increase of the fluorescence intensity of the Edans group due to a decreased FRET efficiency following the release of the Dabcyl-linked peptide quencher fragment. The fluorescent intensity was monitored in a Cary Eclipse fluorescence spectrophotometer (Varian) using wavelengths of 360 nm and 500 nm for the excitation and emission, respectively. The experiments were performed with the same buffer used to store the enzyme (sodium phosphate 10 mM, pH 7.4, sodium chloride 10 mM, DTT 1 mM, EDTA 0.5 mM).

To determine the kinetic parameters, Kₘ and k_{cat}, initial rate measurements were performed by incubating the enzyme with the substrate at room temperature. In a microcuvette with a final volume of 120 μL, the reaction was initiated by adding the protease (final concentration 1 μM) to a solution of substrate at final concentration of 0-120 μM.

### Example 5

### Inhibition Kinetics of Recombinant SARS-Associated 3CL^{pro} Protease

Inhibition assays were performed under the same conditions at increasing concentration of inhibitor. Protease (final concentration 1 μM) was incubated for 20 minutes at room temperature with the inhibitor (final concentration 0-150 μM) and the reaction was initiated adding substrate to a final concentration of 5 μM. Inhibition constants, Kᵢ, were obtained as adjustable parameters through non-linear square fitting of the initial enzymatic rates as a function of the inhibitor concentration according to the following equation:

$$\frac{{v}_{I}}{{v}_{0}} = 1 - \frac{{[ I ]}_{T} + {[ E ]}_{T} + {K}_{i} - \sqrt{{( {[ I ]}_{T} + {[ E ]}_{T} + {K}_{i} )}^{2} - {{4 [ I ]}_{T} [ E ]}_{T}}}{{2 [ E ]}_{T}}$$

where v₁ and v₀ are the initial rate at a given inhibitor concentration and at zero inhibitor concentration, respectively, [I]_{T} is the total inhibitor concentration and [E]_{T} is the total enzyme concentration. The reversibility of the inhibition was confirmed by dilution experiments.

Table 16 shows the results obtained for nine different members of the family of SARS-associated coronavirus protease 3CL^{pro} inhibitors described in this disclosure.

TABLE 16
Inhibition of SARS Associated Coronavirus
Protease 3CL^{pro} by Boronic Acids

| Compound Name | Kᵢ^{app} (μM) |
|---|---|
| FL-061 | - |
| FL-062 | - |
| FL-063 | - |
| FL-064 | - |
| FL-065 | - |
| FL-067 | - |
| FL-068 | - |
| FL-069 | - |
| FL-071 | - |
| FL-072 | - |
| FL-073 | - |
| FL-074 | - |
| FL-075 | - |
| FL-078 | 6.7 ± 0.9 |
| FL-079 | 23.7 ± 3.0 |
| FL-080 | 62 ± 6 |
| FL-081 | 179 ± 70 |
| FL-082 | 158 ± 109 |
| FL-083 | 104 ± 38 |
| FL-084 | > 1000 μM |
| FL-085 | 95 ± 30 |
| FL-086 | > 1000 μM |
| FL-087 | 66 ± 2 |
| FL-088 | 63 ± 14 |
| FL-089 | 241 ± 85 |
| FL-090 | > 1000 μM |
| FL-092 | 15.5 ± 6.5 |
| FL-094 | 39 ± 5 |
| FL-095 | 91.8 ± 8.8 |
| FL-096 | 137 ± 61 |
| FL-097 | 153 ± 58 |
| FL-099 | > 1000 μM |
| FL-100 | 190 ± 100 |
| FL-101 | 21.7 ± 3.27 |
| FL-103 | 39.1 ± 2.1 |
| FL-104 | 18.0 ± 1.0 |
| FL-106 | 8.5 ± 0.5 |
| FL-107 | 6.9 ± 0.3 |
| FL-108 | 15.0 ± 7.0 |
| FL-109 | 61.5 ± 19.7 |
| FL-110 | 36.95 ± 9.7 |
| FL-113 | - |
| FL-114 | 49.8 ± 4.4 |
| FL-132 | - |
| FL-133 | > 1000 μM |
| FL-136 | 8.0 ± 1.7 |
| FL-139 | 73.3 ± 38.0 |
| FL-141 | 39.3 ± 11.7 |
| FL-148 | 21.5 ± 8.7 |
| FL-149 | 313 ± 170 |
| FL-151 | 16.6 ± 6.0 |
| FL-156 | 33.04 ± 9.4 |
| FL-164 | 79.6 ± 14.1 |
| FL-165 | 50.6 ± 21.5 |
| FL-166 | 0.022 ± 0.01 |
| FL-167 | 10.2 ± 2.2 |
| FL-169 | 32.7 ± 2.5 |
| FL-170 | 10.6 ± 7.5 |
| FL-171 | 3.53 ± 3.0 |
| FL-172 | 3.5 ± 3.9 |
| FL-1010 | 84 ± 45 |
| FL-1201 | 35.8 ± 11.5 |

### Example 6

### Binding Energetics of Inhibitors

The binding of the various boronic acids of the present invention to the SARS-associated coronavirus protease 3CL^{pro} was measured by isothermal titration calorimetry and the results are set forth in Table 17 below. Isothermal titration calorimetry does not only measure the binding affinity of inhibitors but also dissects the enthalpic and entropic components to binding, thus allowing identification of the forces involved in the association reaction. In general a binding reaction characterized by a favorable enthalpy change indicates that the inhibitor establishes strong interactions with the target, whereas an inhibitor characterized by unfavorable binding enthalpy is driven by non-specific hydrophobic interactions, i.e. a tendency to escape water rather than a strong attraction to the target (Velazquez-Campoy et al, 2001; Luque and Freire, 2002; Ohtaka et al, 2002).

TABLE 17
Binding Enthalpy of Selected Compounds to 3CL^{pro}

| | ΔH |
|---|---|
| Compound | cal/mol |
| FL-078 | −4900 |
| FL-079 | −1700 |
| FL-106 | −5300 |
| FL-136 | −9200 |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Numerous references, including patents, patent applications, protocols and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entirety and to the same extent as if each reference was individually incorporated by reference.

1. A compound described by formula (1): 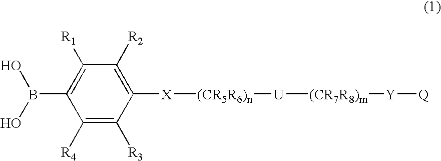
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
R₅ through R₈ each independently represents hydrogen, C₁₋₆ alkyl, C₃₋₇ cycloalkyl, benzyl, or the carbon and attached two Rᵢs, together form C₃₋₇ cycloalkyl;
m and n is each independently 0 or 1;
X and Y is each independently —CO—O—, —O—CO—, —CO—NH—, —NH—CO—, —CH═CH—, —NH—CH₂—, —CH₂—NH—, —SO₂—O—, —O—SO₂—, —SO₂—NH—, —NH—SO₂— or —N═N—;
U represents —(CH₂)ᵢ or —(CH═CH)ⱼ(wherein i=0, 1, 2, or 3 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —C₁H₂—); and
the heterocyclic alkene means a cyclic alkene as defined above, wherein one, two, or all three rings contain(s) one or more S, N or O atom(s);
with the proviso that,
(1) when R₁ through R₄ are H, X is not a —NH—CH₂— or —CH₂—NH—;
(2) when R₁=R=H, X is not —CO—NH—;
(3) when Q is 3-boronophenyl, Y is not —CO—NH or —SO₂—NH—;
(4) when X=—CO—O—, Y=—O—CO—, m=0, n=0 and U is a 1,4-benzene, a 1,4-benzocyclic alkene or a 1,4-benzoheterocyclic alkene, Q is not 4-boronophenyl, which may be substituted with one or more substituents R₁₃;
(5) when R₁ through R₈ are H, X=—CO—O—, m=n=1, U=1,4-benzene, Y=—O—CO—, Q is not 4-boronophenyl;
(6) when R₁ through R₈ are H, X=—CO—O—, m=n=1, U=1,3-benzene, Y=—O—CO—, Q is not 4-boronophenyl;
(7) when R₁ through R₈ are H, X=—CO—O—, m=n=1, U=1,2-benzene, Y=—O—CO—, Q is not 4-boronophenyl;
(8) when R₁ through R₄ are H, X=—NH—SO₂—, m=n=0, U=4-methoxy-1,3-benzene, Y=—N═N—, Q is not 4-(dimethylamino)-1-naphthalenyl; and
(9) when R₁ is amino, R₂ through R₄ are H, X═Y=—N═N—, m=n=0, U is 1,4-naphthalene, Q is not 3-boronophenyl.

2. A compound described by formula (2): 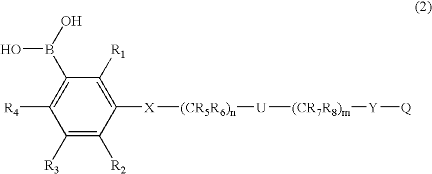
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, —SO₂NH₂;
R₅ through R₈ each independently represents hydrogen, C₁₋₆ alkyl, C₃₋₇ cycloalkyl, benzyl, or the carbon and attached two Rᵢs, they together form C₃₋₇ cycloalkyl;
m and n is each independently 0 or 1;
X and Y is each independently —CO—O—, —O—CO—, —CO—NH—, —NH—CO—, —CH═CH—, —NH—CH₂—, —CH₂—NH—, —SO₂—O—, —O—SO₂—, —SO₂—NH—, —NH—SO₂— or —N═N—;
U represents —(CH₂)ᵢ or —(CH═CH)ⱼ(wherein i=0, 1, 2, or 3 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
the heterocyclic alkene means a cyclic alkene as defined above, wherein one, two, or all three rings contain(s) one or more S, N or O atom(s);
with the proviso that,
(1) when R₁ through R₄ represent hydrogen, X is not —NH—CO—, —NH—SO₂—, —NH—CH₂— or —CH₂—NH—;
(2) when Q is 3-boronophenyl, Y is not —CO—NH or —SO₂—NH—;
(3) when X=—CO—O—, Y=—O—CO—, m=0, n=0 and U is a 1,3-benzene, 1,4-benzene, a 1,3-benzocyclic alkene, a 1,4-benzocyclic alkene, a 1,3-benzoheterocyclic alkene or a 1,4-benzoheterocyclic alkene, Q is not 4-boronophenyl, which may be substituted with one or more substituents R₁₃;
(4) when R₄ is borono, R₁ through R₃ and R₅ through R₈ are H, X=—CO—NH—, Y=—CO—O—, m=n=1, U=—CH₂—CH₂—CH₂—, Q is not 2,5-dioxo-1-pyrrolidinyl; and
(5) when R₃ is borono, R₁, R₂ and R₅ through R₈ are H, X=—CO—NH—, Y=—CO—O—, m=n=1, U=—C₁H₂—C₁H₂—CH₂—, Q is not 2,5-dioxo-1-pyrrolidinyl;

3. A compound described by formula (3): 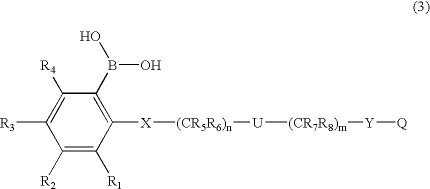
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, —SO₂NH₂;
R₅ through R₈ each independently represents hydrogen, C₁₋₆ alkyl, C₃₋₇ cycloalkyl, benzyl, or the carbon and attached two Rᵢs, they together form C₃₋₇ cycloalkyl;
m and n is each independently 0 or 1;
X and Y is each independently —CO—O—, —O—CO—, —CO—NH—, —NH—CO—, —CH═CH—, —NH—CH₂—, —CH₂—NH—, —SO₂—O—, —O—SO₂—, —SO₂—NH— or —NH—SO₂—;
U represents —(CH₂)ᵢ or —(CH═CH)ⱼ(wherein i=0, 1, 2 or 3 and j=0 or 1), —CHR₉— [wherein R represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₁ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO— or —CH₂—); and
each heterocyclic alkene is independently a cyclic alkene as defined above, wherein one, two, or all three rings contain(s) one or more S, N or O atom(s);
with the proviso that,
(1) when R₁ through R₄ are H, X is not —CH₂—NH—, and
(2) when Q is 3-boronophenyl, Y is not —CO—NH or —SO₂—NH—.

4. A compound described by formula (4): 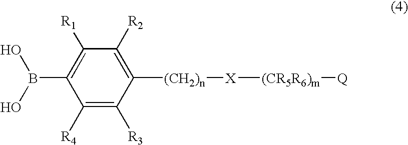
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0 or 1;
X is —CO—O—, —CH═CH—CO—O—, —O—CO—, —CO—NH—, —CH═CH—CO—NH—, —NH—CO—, —CH═N—, —CH═CH—, —NH—CH₂—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁, represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(CO)R₂)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N or O atom(s);
with the proviso that,
(1) when m=0 and Q is 3-boronophenyl, X is not —CO—NH or —SO₂—NH—;
(2) when R₁ through R₄ are H, m=n=0, X is not —CO—O—;
(3) when R₁=R₂=H, X is not —CO—NH—;
(4) when X=—CO—O—, R₅=H, R₆=H, m=1 and n=0, Q is not a benzene, a benzocyclic alkene or a benzoheterocyclic alkene, which may be substituted with one or more substituents R₁₃;
(5) when X=—CO—O—, m=0 and n=0, Q is not a benzene, a benzocyclic alkene or a benzoheterocyclic alkene, which may be substituted with one or more substituents R₁₃;
(6) when R₁ through R₄ are H, X=—CO—O— and m=n=0, Q is not 2,5-dioxo-1-pyrrolidinyl;
(7) when R₁ through R₄ are H, X=—CO—O— and m=n=0, Q is not cyclohexyl;
(8) when R₁ through R₄ are H, X=—O—CO— and m=n=0, Q is not cyclopropyl;
(9) when R₁ through R₄ are H, X=—NH—SO₂—, m=n=0, Q is not 4-(4,5-dihydro-3-phenyl-1H-pyrazol-1-yl)phenyl;
(10) when R₁ through R₄ are H, X=—NH—SO₂—, m=n=0, Q is not 5-(dimethylamino)-1-naphthalenyl;
(11) when R₁ through R₄ are H, X=—NH—CH₂— and m=n=0, Q is not phenyl;
(12) when R₁ through R₄ are H, X=—NH—CO—, m=n=0, Q is not phenyl;
(13) when R₁ through R₄ are H, X=—NH—CO— and m=n=0, Q is not phenyl;
(14) when R₁ through R₄ are H, X=—NH—CO—, m=n=0, Q is not 4-chloro-3-(4-methyl-1-piperazinyl)phenyl;
(15) when R₁ through R₄ are H, X=—NH—CO—, m=n=0, Q is not 4-methoxy-3-(4-methyl-1-piperazinyl)phenyl;
(16) when R₁ through R₄ are H, X=—NH—CO—, m=n=0, Q is not 3-methoxy-4-(4-methyl-1-piperazinyl)phenyl;
(17) when R₁, R₃ through R₆ are H, R₂ is methoxyl, X=—NH—CO—, m=2, n=0, Q is not phenyl;
(18) when R₁ through R₄ are H, X=—N═N—, m=n=0, Q is not 4-(dimethylamino)phenyl;
(19) when R₁ is amino, R₂ through R₄ are H, X=—N═N—, m=n=0, Q is not 1-naphthalenyl;
(20) when R₁ is amino, R through R₄ are H, X=—N═N—, m=n=0, Q is not 4-carboxyphenyl; and
(21) when R₁ through R₄ are H, X=—N═N—, m=n=0, Q is not 2-hydroxy-1-naphthalenyl).

5. A compound described by formula (5): 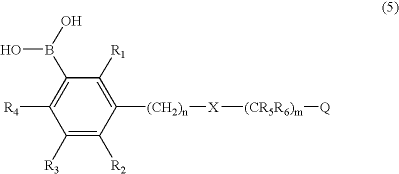
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0 or 1;
X is —CO—O—, —CH═CH—CO—O—, —O—CO—, —CO—NH—, —CH═CH—CO—NH—, —NH—CO—, —CH═N—, —NH—CH₂—, —CH₂—NH—, —SO₂—O—, —O—SO₂—, —SO₂—NH— or —NH—SO₂—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N or O atom(s);
with the proviso that,
(1) when m=0 and Q is 3-boronophenyl, X is not —CO—NH or —SO₂—NH—;
(2) when R₂ and R₃ are H, m=n=0, X is not —CH═CH—;
(3) when m=0, n=0 and Q is a benzene, a benzocyclic alkene or a benzoheterocyclic alkene, which may be substituted with one or more substituents R₁₃, X is not —CO—O—;
(4) when m=0, n=0 and Q is a benzene, a benzocyclic alkene or a benzoheterocyclic alkene, which may be substituted with one or more substituents R₁₃, X is not —NH—CO—;
(5) when m=0, n=0 and Q is a benzene, a benzocyclic alkene or a benzoheterocyclic alkene, which may be substituted with one or more substituents R₁₃, X is not —O—CO—;
(6) when R₅ and R₆=H, m=1, n=0 and Q is a benzene, a benzocyclic alkene or a benzoheterocyclic alkene, which may be substituted with one or more substituents R₁₃, X is not —CO—O—;
(7) when R₁ through R₄ are H, X=—CO—NH—, m=n=0, Q is not phenyl;
(8) when R₁ and R₂ are H, R₃ is F, R₄ is methyl, X=—CO—NH—, m=n=0, Q is not cyclopropyl;
(9) when R₁ through R₆ are H, X=—CO—NH—, m=3, n=0, Q is not phenyl;
(10) when R₁ through R₆ are H, X≡CH₂—CH—, m=1, n=0, Q is not phenyl;
(11) when R₁ through R₄ are H, X=—NH—CH₂—, m=0 and n=1, Q is not phenyl;
(12) when R₁ through R₄ are H, X=—NH—CH₂—, m=0 and n=0, Q is not pentafluorophenyl;
(13) when R₁ through R₄ are H, X=—CH═CH—, m=0 and n=0, Q is not phenyl; and
(14) when R₁ through R₄ are H, X=—CH═CH—, m=0 and n=0, Q is not 2-boronophenyl.

6. A compound described by formula (6): 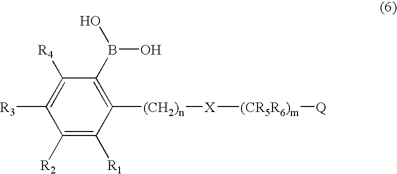
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, —SO₂NH₂;
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0 or 1;
X is —CO—O—, —CH═CH—CO—O—, —O—CO—, —CO—NH—, —CH═CH—CO—NH—, —NH—CO—, —CH═N—, —CH═CH—, —NH—CH₂—, —CH₂—NH—, —CH₂—O—, —SO₂—O—, —O—SO₂—, —SO₂—NH—, or —NH—SO₂—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N or O atom(s);
with the proviso that,
(1) when R₁ through R₄ are H, X is not —CH₂—NH—;
(2) when m=0 and Q is 3-boronophenyl, X is not —CO—NH or —SO₂—NH—;
(3) when R₁ through R₄ are H, X=—NH—CO—, m=0 and n=1, Q is not phenyl;
(4) when R₁ through R₄ are H, X=—NH—CO—, m=0 and n=0, Q is not phenyl;
(5) when R₁ through R₄ are H, X=—NH—CH₂—, m=0 and n=1, Q is not phenyl;
(6) when R₁ through R₆ are H, X=—NH—CH₂—, m=2 and n=1, Q is not phenyl;
(7) when R₁ through R₄ are H, X=—NH—CH₂—, m=0 and n=1, Q is not 10-(hydroxymethyl)-9-anthracenyl;
(8) when R₁ through R₄ are H, X=—NH—CH₂—, m=n=0, Q is not phenyl;
(9) when R₁ through R₈ are H, X=—CH₂—NH—, m=n=1, U is 1,3-benzene, Y=Z=—NH—CH₂—, Q is not 2-boronophenyl; and
(10) when R₁ through R₆ are H, X=—CO—O—, m=1 and n=0, Q is not 4-methoxyphenyl.

7. A compound described by formula (7): 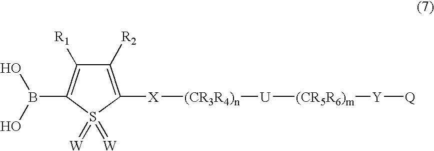
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
R₃ through R₆ each independently represents hydrogen, C₁₋₆ alkyl, C₃₋₇ cycloalkyl, benzyl, or the carbon and attached two Rᵢs, they together form C₃₋₇ cycloalkyl;
m or n is each independently 0 or 1;
X is —CO—O—, —CH₂—O—CO—, —CO—NH—, —CH₂—O—NH—, —CH₂—CH₂, —CH═CH—, —CH₂—O—, —CH═N—, or —CH₂—NH—;
Y is —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ or —(CH═CH)ⱼ (wherein i=0, 1, 2 or 3 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N-(wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N or O atom(s).

8. A compound described by formula (8): 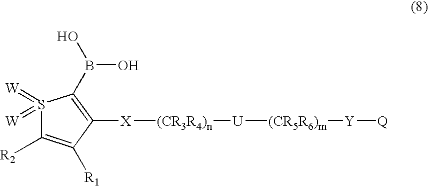
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
R₃ through R₆ each independently represents hydrogen, C₁₋₆ alkyl, C₃₋₇ cycloalkyl, benzyl, or the carbon and attached two Rᵢs, they together form C₃₋₇ cycloalkyl;
m or n is each independently 0 or 1;
X is —CO—O—, —CH₂—O—CO—, —CO—NH—, —CH₂—O—NH—, —CH₂—CH₂, —CH═CH—, —CH₂—O—, —CH═N—, or —CH₂—NH—;
Y is —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ or —(CH═CH)ᵢ (wherein i=0, 1, 2 or 3 and j=0 or 1), —CHR₉— [wherein R₅ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₆₋₄ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N or O atom(s).

9. A compound described by formula (9): 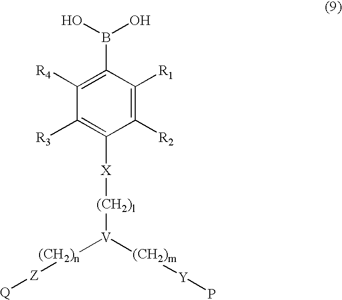
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, —SO₂NH₂;
l, m, and n is each independently 0, 1, or 2;
X, Y, and Z is each independently —CO—O—, —O—CO—, —CO—NH—, —NH—CO—, —CH═CH—, —NH—CH₂—, —CH═N—, —CH₂—NH—, —SO₂—O—, —O—SO₂—, —SO₂—NH—, —NH—SO₂— or —N═N—;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N or O atom(s);
with the proviso that,
when P or Q is 3-boronophenyl, Y or Z is not —CO—NH or —SO₂—NH—.

10. A compound described by formula (10): 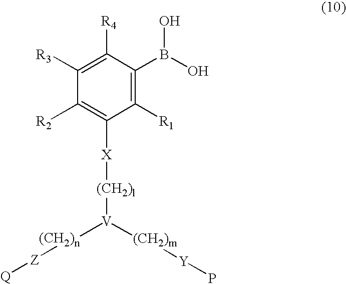
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —CH═N—, —SO₂—O—, —O—SO₂—, —SO₂—NH—, —NH—SO₂— or —N═N—. Y or Z is not —CO—NH or —SO₂—NH—;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₆₋₄ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO— or —CH₂—); and
each heterocyclic alkene is independently a cyclic alkene as defined above, wherein one, two, or all three rings contain(s) one or more S, N or O atom(s);
with the proviso that,
when R₁ through R₄ represent hydrogen, X is not —NH—CO— or —NH—SO₂—.

11. A compound described by formula (11): 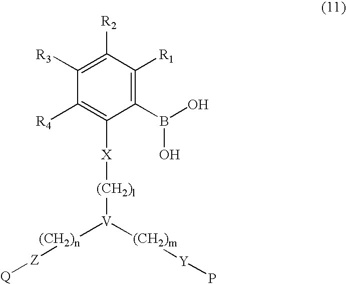
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —CH═N—, —SO₂—O—, —O—SO₂—, —SO₂—NH—, —NH—SO₂— or —N═N—;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₁ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N or O atom(s);
with the proviso that,
(1) when P or Q is 3-boronophenyl, Y or Z is not —CO—NH or —SO₂—NH—; and
(2) when R₁ through R₄ are H, X=—CH₂—NH—, l=m=n=2, V is a nitrogen, Y=Z=—NH—CH₂—, P and Q is not 2-boronophenyl at the same time.

12. A compound described by formula (12): 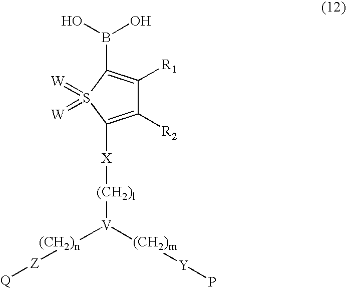
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N or O atom(s);
with the proviso that,
when P or Q is 3-boronophenyl, Y or Z is not —CO—NH or —SO₂—NH—.

13. A compound described by formula (13): 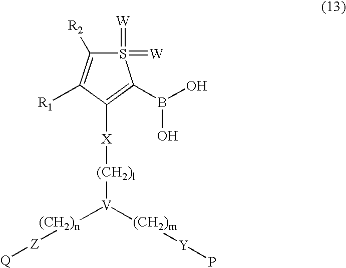
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N or O atom(s);
with the proviso that,
when P or Q is 3-boronophenyl, Y or Z is not —CO—NH or —SO₂—NH—.

14. A compound described by formula (14): 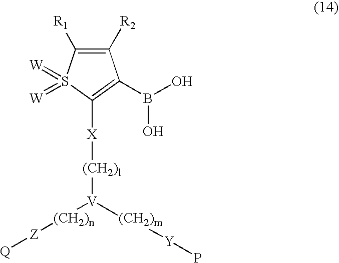
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
l, m, and n is each independently 0, 1, or 2;
X, Y, and Z is each independently —CO—O—, —O—CO—, —CO—NH—, —NH—CO—, —CH═CH—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
V represents a nitrogen, —CH₂—CH═, —CH₂—CH₂—CH═, —CHCH₃—CH═, a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₁ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N or O atom(s);
with the proviso that,
when P or Q is 3-boronophenyl, Y or Z is not —CO—NH or —SO₂—NH—.

15. A compound described by formula (15): 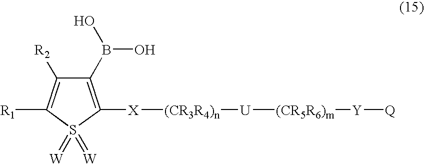
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen, C₁₋₆ alkyl or benzyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —C(CH₃)═NOH, —C(OH)═NOH, —CONHOH, —SO₃H, —SO₂CH₃, or —SO₂NH₂;
R₃ through R₆ each independently represents hydrogen, C₁₋₆ alkyl, C₃₋₇ cycloalkyl, benzyl, or the carbon and attached two Rᵢs, they together form C₃₋₇ cycloalkyl;
m or n is each independently 0 or 1;
X is —CO—O—, —CH₂—O—CO—, —CO—NH—, —CH₂—O—NH—, —CH₂—CH₂, —CH═CH—, —CH₂—O—, —CH═N—, or —CH₂—NH—;
Y is —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ or —(CH═CH)ᵢ (wherein i=0, 1, 2 or 3 and j=0 or 1), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₅₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
Q represents —CH₂CHR₁₁COR₁₂ or —CHR₁₁COR₁₂ [wherein R₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, R₁₄R₁₅N— (wherein R₁₄ and R₁₅ are each independently hydrogen or C₁₋₆ alkyl), R₁₄R₁₅R₁₆N⁺G⁻ (wherein R¹⁴, R₁₅ and R₁₆ are each independently hydrogen, C₁₋₆ alkyl or benzyl, G represents halogen, SO₄ or BF₄), trifluoromethyl, trifluoromethoxy, halogen, cyano, acetyl, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NH₂, —CONR₁₉OH or —CHR₂₀N(COR₂₁)OH (wherein R₁₉ through R₂₁ each independently represents a hydrogen, C₁₋₆ alkyl, trifluoromethyl or benzyl);
each cyclic alkene is independently a structure containing 1, 2 or 3 rings, each ring containing 5, 6 or 7 carbon atoms and at least one double bond; one, two, or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N or O atom(s).

16. A pharmaceutical composition comprising the compound of claim 1 and a pharmaceutically acceptable carrier.

17. A pharmaceutical composition comprising the compound of claim 2 and a pharmaceutically acceptable carrier.

18. A pharmaceutical composition comprising the compound of claim 3 and a pharmaceutically acceptable carrier.

19. A pharmaceutical composition comprising the compound of claim 4 and a pharmaceutically acceptable carrier.

20. A pharmaceutical composition comprising the compound of claim 5 and a pharmaceutically acceptable carrier.

21. A pharmaceutical composition comprising the compound of claim 6 and a pharmaceutically acceptable carrier.

22. A pharmaceutical composition comprising the compound of claim 7 and a pharmaceutically acceptable carrier.

23. A pharmaceutical composition comprising the compound of claim 8 and a pharmaceutically acceptable carrier.

24. A pharmaceutical composition comprising the compound of claim 9 and a pharmaceutically acceptable carrier.

25. A pharmaceutical composition comprising the compound of claim 10 and a pharmaceutically acceptable carrier.

26. A pharmaceutical composition comprising the compound of claim 11 and a pharmaceutically acceptable carrier.

27. A pharmaceutical composition comprising the compound of claim 12 and a pharmaceutically acceptable carrier.

28. A pharmaceutical composition comprising the compound of claim 13 and a pharmaceutically acceptable carrier.

29. A pharmaceutical composition comprising the compound of claim 14 and a pharmaceutically acceptable carrier.

30. A pharmaceutical composition comprising the compound of claim 15 and a pharmaceutically acceptable carrier.

31. A method for treating infection caused by coronavirus comprising administering to a subject suffering from such an infection an effective amount of a boron-containing compound.

32. The method of claim 31 where in the boron-containing compound is described by formula (A) 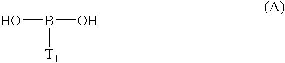
wherein T₁ comprises a ring structure or any other organic functional group; and
B is boron.

33. The method of claim 31 wherein the boron-containing compound is described by formula (B) 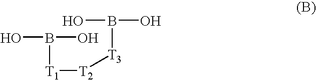
wherein T₁ and T₃ each comprises a ring structure or any other organic functional group; and T₂ is a linker.

34. The method of claim 31, wherein the boron-containing compound is a multi-functional boronic acid.

35. The method of claim 31, wherein the boron-containing compound is a bi-functional boronic acid.

36. The method of claim 31, wherein the boron-containing compound is described by formula (1) 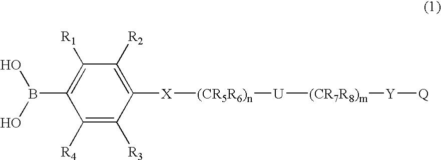
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ through R₈ each independently represents hydrogen, C₁₋₆ alkyl, C₃₋₇ cycloalkyl, benzyl or the carbon and attached two Rᵢs, they together form C₃₋₇ cycloalkyl;
m and n are each independently 0, 1, or 2;
X and Y each independently represents —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ⱼOR₁₈— (wherein R₁₈ is hydrogen or C₁₋₆ alkyl, and j is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—);
each heterocyclic alkene is independently a cyclic alkene as defined above, wherein one, two, or all three rings contain(s) one or more S, N, O, P or Se atom(s).

37. The method of claim 31, wherein the boron-containing compound is desecribed by formula (2) 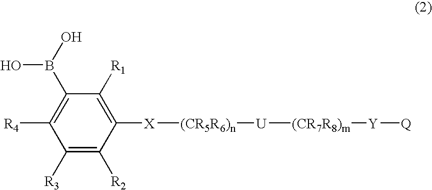
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ through R₈ each independently represents hydrogen, C₁₋₆ alkyl, C₃₋₇ cycloalkyl, benzyl or the carbon and attached two Rᵢs, they together form C₃₋₇ cycloalkyl;
m and n are each independently 0, 1, or 2;
X and Y each independently represents —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR₉— [wherein R₉ represents C₁₋₆alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ⱼOR₁₈— (wherein R₁₈ is hydrogen or C₁₋₆ alkyl, and j is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

38. The method of claim 31, wherein the boron-containing compound is described by formula (3) 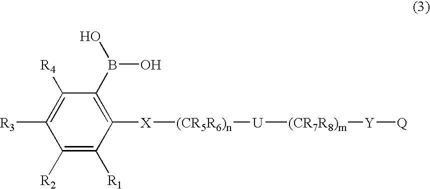
wherein
R₁ through R₄ each independently represents hydrogen, C₁₋₁ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₁ alkyl), —O(CH₂)ₖOR₁₈— (wherein R₁₈ is hydrogen or C₁₋₁ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ through R₈ each independently represents hydrogen, C₁₋₁ alkyl, C₃₋₇ cycloalkyl, benzyl or the carbon and attached two Rᵢs, they together form C₃₋₇ cycloalkyl;
m and n are each independently 0, 1, or 2;
X and Y each independently represents —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₁ alkyl, C₃₋₇ cycloalkyl, C₁₋₁ alkoxy, R₁₄R₁₅N— (wherein R₁₄ and R₁₅ are each independently hydrogen or C₁₋₁ alkyl), R₁₄R₁₅R₁₆N⁺G⁻ (wherein R₁₄, R₁₅ and R₁₆ are each independently hydrogen, C₁₋₁ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₁ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ⱼOR₁₈— (wherein R₁₈ is hydrogen or C₁₋₆ alkyl, and j is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

39. The method of claim 31, wherein the boron-containing compound is desecribed by formula (4) 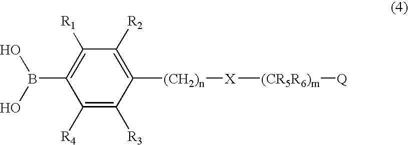
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, formyl;
m and n is each independently 0, 1 or 2;
X is —O—, —NH—, —S—, —SO₂—, —CO—, —CH₂—, —CH═CH—CO—O—, —CH═CH—CO—NH—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

40. The method of claim 31, wherein the boron-containing compound is desecribed by formula (5) 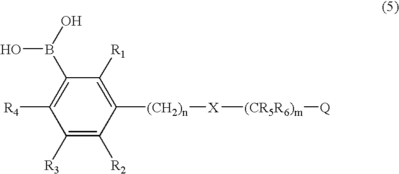
wherein R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₉ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, formyl;
m and n is each independently 0, 1 or 2;
X is —O—, —NH—, —S—, —SO₂—, —CO—, —CH₂—, —CH═CH—CO—O—, —CH═CH—CO—NH—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

41. The method of claim 31, wherein the boron-containing compound is desecribed by formula (6) 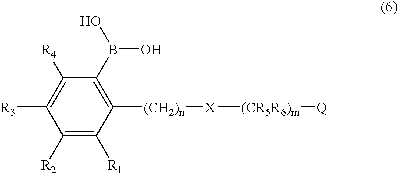
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0, 1 or 2;
X is —O—, —NH—, —S—, —SO₂—, —CO—, —CH₂—, —CH═CH—CO—O—, —CH═CH—CO—NH—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

42. The method of claim 31, wherein the boron-containing compound is described by formula (7) 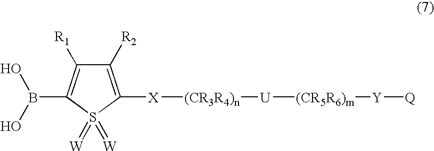
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₃ through R₆ each independently represents hydrogen, C₁₋₆ alkyl, C₃₋₇ cycloalkyl, benzyl or the carbon and attached two Rᵢs, they together form C₃₋₇ cycloalkyl;
m and n is each independently 0 or 1;
X and Y is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5 or 6, and j=0, 1 or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₁ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₁ alkyl or benzyl, G represents halogen, SO₄ or BF₄), trifluoromethyl, trifluoromethoxy, difluoromethoxy, halogen, cyano, borono, nitro, carboxyl, —CH₂COOH, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

43. The method of claim 31, wherein the boron-containing compound is desecribed by formula (8) 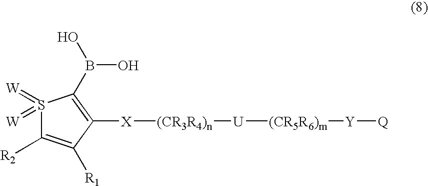
wherein
R₁ and R₂ each independently represents hydrogen, C₁₋₁ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₁ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₃ through R₆ each independently represents hydrogen, C₁₋₆ alkyl, C₃₋₇ cycloalkyl, benzyl or the carbon and attached two Rᵢs, they together form C₃₋₇ cycloalkyl;
m and n is each independently 0 or 1;
X and Y is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5 or 6, and j=0, 1 or 2), —CHR₉— [wherein R₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₁ alkyl or benzyl, G represents halogen, SO₄ or BF₄), trifluoromethyl, trifluoromethoxy, difluoromethoxy, halogen, cyano, borono, nitro, carboxyl, —CH₂COOH, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₁ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₁ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic, one or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

44. The method of claim 31, wherein the boron-containing compound is desecribed by formula (9) 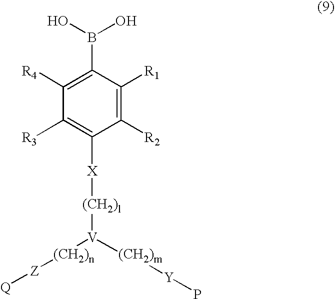
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₁ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m and n is each independently 0, 1 or 2;
X, Y and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene which may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene which may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₁ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₁ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

45. The method of claim 31, wherein the boron-containing compound is desecribed by formula (10) 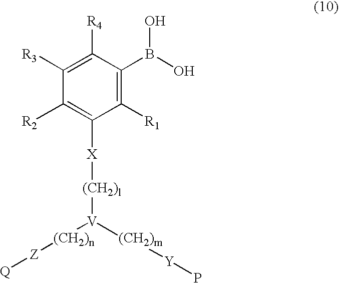
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m and n is each independently 0, 1 or 2;
X, Y and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene which may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene which may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

46. The method of claim 31, wherein the boron-containing compound is desecribed by formula (11) 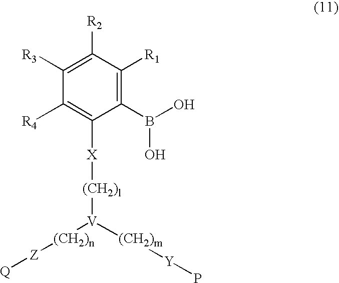
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₉ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m and n is each independently 0, 1 or 2;
X, Y and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene which may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene which may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3); —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

47. The method of claim 31, wherein the boron-containing compound is desecribed by formula (12) 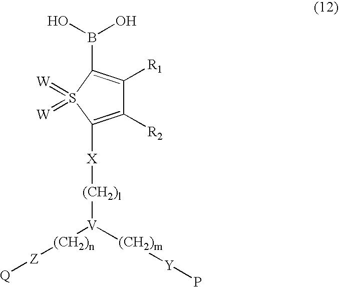
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m, and n is each independently 0, 1, or 2;
X, Y, and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons.
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

48. The method of claim 31, wherein the boron-containing compound is desecribed by formula (13) 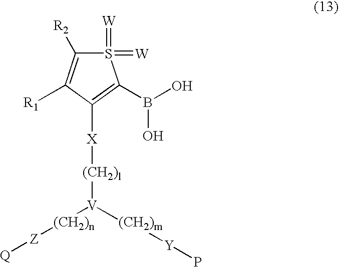
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m, and n is each independently 0, 1, or 2;
X, Y, and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons.
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, n-butoxy, i-butoxy, sec-butoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

49. The method of claim 31, wherein the boron-containing compound is desecribed by formula (14) 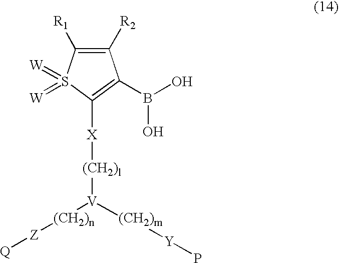
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m, and n is each independently 0, 1, or 2;
X, Y, and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons.
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, n-butoxy, i-butoxy, sec-butoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

50. The method of claim 31, wherein the boron-containing compound is desecribed by formula (15) 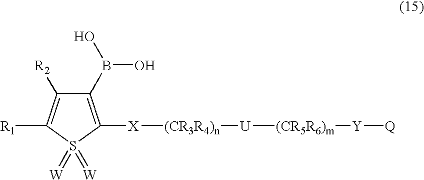
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₃ through R₆ each independently represents hydrogen, C₁₋₆ alkyl, C₃₋₇ cycloalkyl, benzyl or the carbon and attached two Rᵢs, they together form C₃₋₇ cycloalkyl;
m and n is each independently 0 or 1;
X and Y is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5 or 6, and j=0, 1 or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, —CH₂COOH, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

51. The method of claim 31, wherein the coronavirus is a coronavirus having protease(s) that has one or more serine or threonine residue(s) at or near its active site.

52. The method of claim 31, wherein the coronavirus is SARS-associated coronavirus.

53. A method for inhibiting coronavirus protease comprising contacting the coronavirus protease with an effective amount of a boron-containing compound.

54. The method of claim 53 where in the boron-containing compound is described by formula (A) 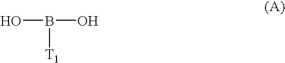
wherein T₁ comprises a ring structure or any other organic functional group; and
B is boron.

55. The method of claim 53 wherein the boron-containing compound is described by formula (B) 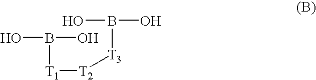
wherein T₁ and T₃ each comprises a ring structure or any other organic functional group; and T₂ is a linker.

56. The method of claim 53, wherein the boron-containing compound is a multi-functional boronic acid.

57. The method of claim 53, wherein the boron-containing compound is a bi-functional boronic acid.

58. The method of claim 53, wherein the boron-containing compound is described by formula (1) 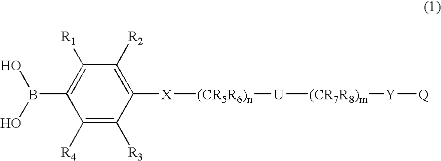
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ through R₈ each independently represents hydrogen, C₁₋₆ alkyl, C₃₋₇ cycloalkyl, benzyl or the carbon and attached two Rᵢs, they together form C₃₋₇ cycloalkyl;
m and n are each independently 0, 1, or 2;
X and Y each independently represents —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ⱼOR₁₈— (wherein R₁₈ is hydrogen or C₁₋₆ alkyl, and j is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond. One, two or all three rings may be aromatic. One or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—);
each heterocyclic alkene is independently a cyclic alkene as defined above, wherein one, two, or all three rings contain(s) one or more S, N, O, P or Se atom(s).

59. The method of claim 53, wherein the boron-containing compound is described by formula (2) 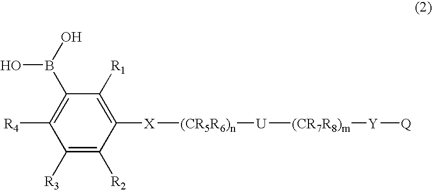
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ through R₈ each independently represents hydrogen, C₁₋₆ alkyl, C₃₋₇ cycloalkyl, benzyl or the carbon and attached two Rᵢs, they together form C₃₋₇ cycloalkyl;
m and n are each independently 0, 1, or 2;
X and Y each independently represents —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR₉— [wherein R₅ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ⱼOR₁₈— (wherein R₁₈ is hydrogen or C₁₋₆ alkyl, and j is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

60. The method of claim 53, wherein the boron-containing compound is described by formula (3) 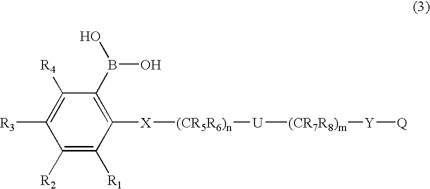
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ through R₈ each independently represents hydrogen, C₁₋₆ alkyl, C₃₋₇ cycloalkyl, benzyl or the carbon and attached two Rᵢs, they together form C₃₋₇ cycloalkyl;
m and n are each independently 0, 1, or 2;
X and Y each independently represents —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5, or 6, and j=0, 1, or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N—(wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₁ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ⱼOR₁₈— (wherein R₁₉ is hydrogen or C₁₋₆ alkyl, and j is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₁ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₁ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₂₁)OH (wherein R₁₉ through R₂₁ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

61. The method of claim 53, wherein the boron-containing compound is described by formula (4) 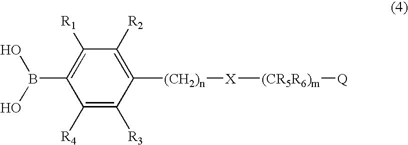
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, formyl;
m and n is each independently 0, 1 or 2;
X is —O—, —NH—, —S—, —SO₂—, —CO—, —CH₂—, —CH═CH—CO—O—, —CH═CH—CO—NH—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

62. The method of claim 53, wherein the boron-containing compound is described by formula (5) 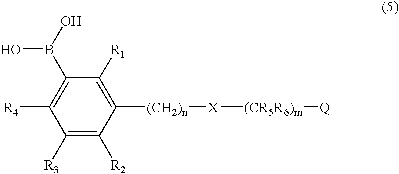
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, formyl;
m and n is each independently 0, 1 or 2;
X is —O—, —NH—, —S—, —SO₂—, —CO—, —CH₂—, —CH═CH—CO—O—, —CH═CH—CO—NH—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
Q represents —CH₂CHR₁₁COR₁₂ or —CHR₁₁ COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

63. The method of claim 53, wherein the boron-containing compound is described by formula (6) 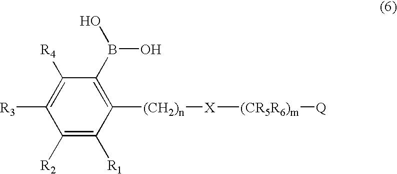
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₅ and R₆ each independently represents hydrogen, C₁₋₆ alkyl, C₃₋₇ cycloalkyl, trifluoromethyl, trifluoromethoxy, halogen, acetyl, carboxyl, CH₂COOH, C₁₋₆ alkoxycarbonyl, phenyl, phenoxy, phenoxycarbonyl, benzoyl, benzyl, benzyloxy, hydroxyl, hydroxymethyl, or formyl;
m and n is each independently 0, 1 or 2;
X is —O—, —NH—, —S—, —SO₂—, —CO—, —CH₂—, —CH═CH—CO—O—, —CH═CH—CO—NH—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

64. The method of claim 53, wherein the boron-containing compound is described by formula (7) 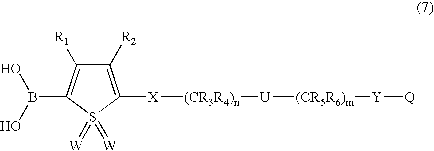
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₃ through R₆ each independently represents hydrogen, C₁₋₆ alkyl, C₃₋₇ cycloalkyl, benzyl or the carbon and attached two Rᵢs, they together form C₃₋₇ cycloalkyl;
m and n is each independently 0 or 1;
X and Y is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5 or 6, and j=0, 1 or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, —CH₂COOH, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

65. The method of claim 53, wherein the boron-containing compound is described by formula (8) 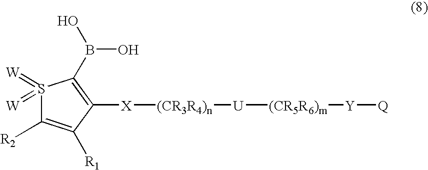
wherein
R₁ and R₂ each independently represents hydrogen, C₁₋₁ alkyl, C₃₋₇ cycloalkyl, C₁₋₆ alkoxy, R₁₄R₁₅N— (wherein R₁₄ and R₁₅ are each independently hydrogen or C₁₋₁ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₃ through R₆ each independently represents hydrogen, C₁₋₆ alkyl, C₃₋₇ cycloalkyl, benzyl or the carbon and attached two Rᵢs, they together form C₃₋₇ cycloalkyl;
m and n is each independently 0 or 1;
X and Y is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5 or 6, and j=0, 1 or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, —CH₂COOH, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic, one or more carbon(s) may be attached to oxygen to form —CO—. If the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

66. The method of claim 53, wherein the boron-containing compound is described by formula (9) 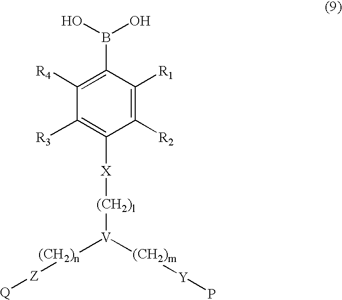
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m and n is each independently 0, 1 or 2;
X, Y and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene which may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene which may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

67. The method of claim 53, wherein the boron-containing compound is described by formula (10) 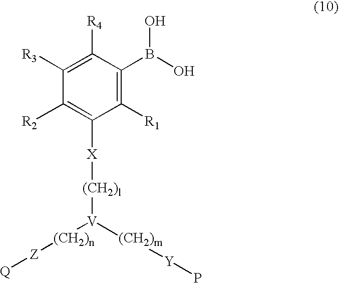
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m and n is each independently 0, 1 or 2;
X, Y and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene which may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene which may be substituted with one or more substituents R₁₃, and
each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

68. The method of claim 53, wherein the boron-containing compound is described by formula (11) 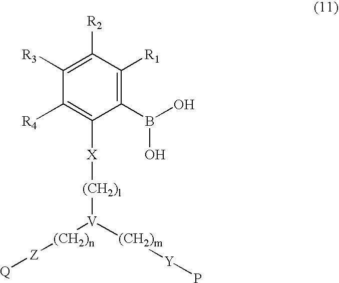
wherein
R₁ through R₄ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m and n is each independently 0, 1 or 2;
X, Y and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene which may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene which may be substituted with one or more substituents R₁₃, and each R₁₃ independently represents hydrogen, C₁₋₁ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

69. The method of claim 53, wherein the boron-containing compound is described by formula (12) 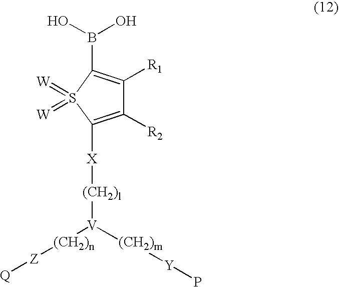
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m, and n is each independently 0, 1, or 2;
X, Y, and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons.
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₁ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₁ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, n-butoxy, i-butoxy, sec-butoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

70. The method of claim 53, wherein the boron-containing compound is described by formula (13) 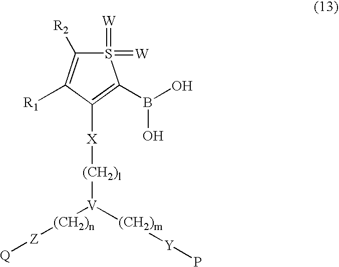
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m, and n is each independently 0, 1, or 2;
X, Y, and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons.
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₆₋₄ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₆₋₄ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

71. The method of claim 53, wherein the boron-containing compound is described by formula (14) 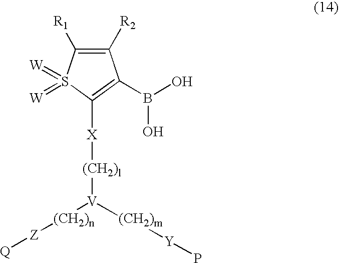
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
l, m, and n is each independently 0, 1, or 2;
X, Y, and Z is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons.
V represents nitrogen, —CH═C═, —CH₂—CH═, —CH₂—CH₂—CH═, —CHCH₃—CH═, a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈—(wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
P and Q each independently represents —CH₂CHR₁₁COR₁₂ or —CHR₁₁COR₁₂ [wherein R₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, n-butoxy, i-butoxy, sec-butoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

72. The method of claim 53, wherein the boron-containing compound is described by formula (15) 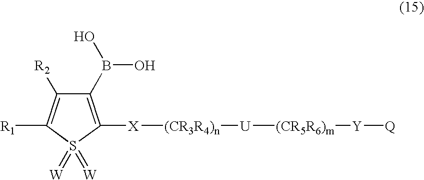
wherein
R₁ and R₂ each independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
R₃ through R₆ each independently represents hydrogen, C₁₋₆ alkyl, C₃₋₇ cycloalkyl, benzyl or the carbon and attached two Rᵢs, they together form C₃₋₇ cycloalkyl;
m and n is each independently 0 or 1;
X and Y is each independently —O—, —NH—, —S—, —SO₂—, —CO—, —CH₂—, —CO—O—, —O—CO—, —CO—NH—, —NH—CO—, —CH₂—CH₂—, —CH═CH—, —O—CH₂—, —CH₂—O—, —NH—CH₂—, —CH═N—, —CH₂—NH—, —SO₂—O—, —O—SO₂—, —SO₂—NH—, —NH—SO₂— or —N═N—;
W is oxygen or lone-pair electrons;
U represents —(CH₂)ᵢ—, —(CH═CH)ⱼ—, —(CH₂CH₂O)ⱼ— or —(CH₂CH₂N)ⱼ— (wherein i=0, 1, 2, 3, 4, 5 or 6, and j=0, 1 or 2), —CHR₉— [wherein R₉ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₀], a C₃₋₇ cycloalkane, a cyclic alkene or a heterocyclic alkene, wherein the cycloalkane, cyclic alkene or heterocyclic alkene may be substituted with one or more substituents R₁₀, and each R₁₀ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, —CH₂COOH, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
Q represents —CH₂CHR₁₁COR₁₂ or —CHR₁₁COR₁₂ [wherein R₁₁ represents C₁₋₆ alkyl, C₃₋₇ cycloalkyl, a cyclic alkene or a heterocyclic alkene, wherein the cycloalkane, cyclic alkene or heterocyclic alkene may be substituted with one or more substituents R₁₃, R₁₂ represents hydroxyl, C₁₋₆ alkoxy, —NR₁₉OH (wherein R₁₉ represents a hydrogen, C₁₋₆ alkyl, C₃₋₇ cycloalkyl, trifluoromethyl, phenyl or benzyl), R₁₄R₁₅N— (wherein R₁₄ and R₁₅ are each independently hydrogen, hydroxyl or C₁₋₆ alkyl)], a C₃₋₇ cycloalkane, a cyclic alkene or a heterocyclic alkene, wherein the cycloalkane, cyclic alkene or a heterocyclic alkene may be substituted with one or more substituents R₁₃, and each R₁₃ independently represents hydrogen, C₁₋₆ alkyl, C₃₋₇ cycloalkyl, C₁₋₆ alkoxy, R₁₄R₁₅N— (wherein R₁₄ and R₁₅ are each independently hydrogen or C₁₋₆ alkyl), R₁₄R₁₅R₁₆N⁺G⁻ (wherein R₁₄, R₁₅ and R₁₆ are each independently hydrogen, C₁₋₆ alkyl or benzyl, G represents halogen, SO₄ or BF₄), trifluoromethyl, trifluoromethoxy, difluoromethoxy, halogen, cyano, borono, nitro, carboxyl, C₁₋₆ alkylcarboxyl, C₁₋₆ alkoxycarbonyl, phenyl, phenoxy, phenoxycarbonyl, benzoyl, benzyl, benzyloxy, hydroxyl, trimethylsilyloxy, diphenyl-t-butylsilyloxy, hydroxymethyl, C₁₋₆ alkylcarbonyl, —CH═NOH, —CH₂NHOH, —C(CH₃)═NOH, —C(OH)═NOH, —SO₃H, —SO₂CH₃, —SO₂NHR₁₇ (wherein R₁₇ is hydrogen or C₁₋₆ alkyl), —O(CH₂)ₖOR₁₈— (wherein R₁₈ is hydrogen or C₁₋₆ alkyl, and k is 1, 2 or 3), —CONR₁₉OH or —CHR₂₀N(COR₁₉)OH (wherein R₁₉ and R₂₀ each independently represents a hydrogen, C₁₋₆ alkyl, C₃₋₇ cycloalkyl, trifluoromethyl, phenyl or benzyl);
each cyclic alkene is independently a structure containing 1, 2 or 3 rings, each ring containing 5, 6 or 7 carbon atoms and at least one double bond; one, two or all three rings may be aromatic; one or more carbon(s) may be attached to oxygen to form —CO—; if the cyclic alkene contains more than one ring, the ring may be fused, connected by a bond, or connected by a linker L (wherein L includes —O—, —NH—, —S—, —SO₂—, —CO—, or —CH₂—); and
each heterocyclic alkene is independently a cyclic alkene as defined above, wherein one, two, or all three rings contain(s) one or more S, N, O, P or Se atom(s).

73. The method of claim 53, wherein the coronavirus is a coronavirus having protease(s) that has one or more serine or threonine residue(s) at or near its active site.

74. The method of claim 53, wherein the coronavirus is SARS-associated coronavirus.

75. A method for detecting coronavirus in a test sample comprising contacting the sample with an effective amount of a boron-containing compound.

76. The method of claim 75 where in the boron-containing compound is described by formula (A) 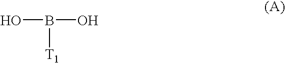
wherein T₁ comprises a ring structure or any other organic functional group; and
B is boron.

77. The method of claim 75 wherein the boron-containing compound is described by formula (B) 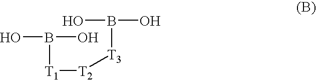
wherein T₁ and T₃ each comprises a ring structure or any other organic functional group; and T₂ is a linker.

78. The method of claim 75, wherein the boron-containing compound is a multi-functional boronic acid.

79. The method of claim 75, wherein the boron-containing compound is a bi-functional boronic acid.
